(19) United States
(12) Patent Application Publication
(10) Pub. No.: US 2024/0209085 A1
(43) Pub. Date: Jun. 27, 2024

(54) ADVERSE EFFECTS-MITIGATING ADMINISTRATION OF A BISPECIFIC CONSTRUCT BINDING TO CD33 AND CD3

(71) Applicant: AMGEN INC., Thosand Oaks, CA (US)

(72) Inventors: Sophia K. Khaldoyanidi, Thousand Oaks, CA (US); Dirk Nagorsen, Thousand Oaks, CA (US)

(21) Appl. No.: 17/927,071

(22) PCT Filed: May 31, 2021
(86) PCT No.: PCT/US21/35069

§ 371 Date: Nov. 22, 2022

Related U.S. Application Data
(60) Provisional application No. 63/044,833, filed on Jun. 26, 2020.
(60) Provisional application No. 63/032,302, filed on May 29, 2020.

(51) Int. Cl. C07K 16/28; A61K 39/00; A61P 35/02

(52) U.S. Cl. CPC C07K 16/2809; A61P 35/02; C07K 16/2803; A61K 2039/545; C07K 2317/31; C07K 2317/622

(57) ABSTRACT

The present invention provides a bispecific construct comprising a first binding domain specifically binding to a target such as CD33 and a second binding domain specifically binding to an effector such as CD3 for use in a method for the treatment of myeloid leukemia, wherein the construct is administered in one or more treatment cycles of more than 14 days applying a step dosing comprising at least two, preferably steps, wherein the first step is higher than the second step with respect to the previous dosage, and wherein the second step is higher than the optional but preferred third step with respect to the previous dosage, a treatment cycle optionally followed by a period without administration of the construct. Moreover, the invention provides a method for the treatment of myeloid leukemia comprising the administration of a therapeutically efficient amount of such bispecific construct and the use of such bispecific construct for the preparation of a pharmaceutical composition for the treatment of myeloid leukemia.

### FIELD OF THE INVENTION

The present invention relates to a bispecific construct comprising a first binding domain specifically binding to a target such as CD33 and a second binding domain specifically binding to an effector such as CD3, preferably for use in a method for the treatment of acute myeloid leukemia. Moreover, the invention relates to a method for the treatment of acute myeloid leukemia comprising the administration of a therapeutically efficient amount of such bispecific construct and the use of such bispecific construct for the preparation of a pharmaceutical composition for the treatment of acute myeloid leukemia.

### BACKGROUND OF THE INVENTION

Bispecific constructs such as BiTE® (bispecific T cell engager) constructs are recombinant protein constructs made from two flexibly linked antibody derived binding domains. One binding domain of bispecific constructs is specific for a selected tumor-associated surface antigen on target cells; the second binding domain is specific for CD3, a subunit of the T cell receptor complex on T cells. By their particular design BiTE® constructs are uniquely suited to transiently connect T cells with target cells and, at the same time, potently activate the inherent cytolytic potential of T cells against target cells. The first generation of bispecific constructs (see WO 99/54440 and WO 2005/040220) developed into the clinic as blinatumomab and solitomab. These bispecific constructs are administered via continuous intravenous infusion. For example, blinatumomab is administered in B acute lymphoblastic leukemia as 4-week infusing with a lower initial dose in the 1st week and a higher dose in the remaining treatment for the 1st cycle and in all other cycles from start. Before starting a second cycle, there is a treatment-free period of two weeks. A similar administration schema has been used for solitomab which was administered as continuous intravenous infusion over at least 28 days with increasing doses and also a treatment-free period of two weeks between two cycles.

An important further development of the first generation of bispecific constructs was the provision of bispecific constructs binding to a context independent epitope at the N-terminus of the CD3εchain of human and Callithrix jacchus, Saguinus oedipus or Saimiri sciureus (WO 2008/119567). Hence, such bispecific constructs have become versatile means to address so-far unmet therapeutic needs.

One such need is an efficient and safe therapy of Acute Myeloid Leukemia (AML), in particular relapsed or refractory AML (r/r AML), or AML with minimal residual disease (MRD) or myelodysplastic syndrome (MDS). Acute myeloid leukemia, whereof MDS is a typical precursor condition, is the most common form of acute leukemia in adults in the United States (US), with a rising incidence attributed to an aging population, an increase in environmental exposure, and an increase in the population of cancer survivors previously exposed to chemotherapy and therapeutic radiation. In the US, an estimated 21450 new cases of AML and 10920 deaths from AML were expected in 2019 (Siegel et al, 2019). Prognosis of patients with relapsed or refractory AML is poor as no standard salvage therapy exists except in the case of AML with specific mutations such IDH1/2 mutations. Most trials of investigational agents begin in r/r AML and have accrued a wide range of patients with different characteristics. Historical context for outcomes can be used as a reference for the development of future protocols and novel agents. Analysis of such historical context reveals that overall survival and event free survival were modest and decreased with subsequent salvage. Age, cytogenetics, antecedent disease, De novo/therapy-induced AML, duration of first remission, and platelet count were associated with survival. Importantly, in the vast majority of cases, patients, in particular a majority of r/r AML patients, are unable to achieve a sustained second or subsequent remission, i.e. a long-term amelioration or even cure of the disease. Hence, there is a need for further therapeutic means and an optimized use thereof.

CD33 is a sialic-acid-dependent cytoadhesion molecule known as a myeloid differentiation antigen found inter alia on AML blasts in most patients and leukemic stem cells Therefore, CD33 has been identified as a promising marker for myeloid leukemia and a target molecule in the treatment of such diseases. To this end, Mylotarg® (gemtuzumab ozogamcin), a cytotoxic antibiotic linked to a recombinant monoclonal antibody directed against the CD33 antigen present on leukemic myeloblasts, had been approved in the United States for patients with AML through accelerated approval. However, following the drug's failure to demonstrate clinical benefit in the confirmatory trial, and an increased risk of venoocclusive disease observed in the postmarketing setting, the drug was temporarily withdrawn voluntarily by the manufacturer from the United States market. Frequently reported toxicities observed with gemtuzumab ozogamcin included neutropenia and thrombocytopenia, and less frequently reported toxicities included events related to acute infusion-related reactions (anaphylaxis), hepatotoxicity, and veno-occlusive disease. A promising CD33×CD3 bispecific construct has been proposed before for use in the treatment of AML. While first encouraging results indicated clinical efficacy in terms of observed complete remissions, cytokine release syndrome (CRS) is the key toxicity which is typically associated with such bispecific constructs, as the resulting activation of T cells involves the transient release of inflammatory cytokines. During treatment with a CD33×CD3 bispecific construct, signs and symptoms of CRS typically occur within the first 24 hours after initiation of the therapy, and may include pyrexia, rash, chills, hypoxia, dyspnea, tachycardia, headache, nausea, vomiting, hypotension, hypertension, AST and/or ALT elevations, and hyperbilirubinemia. CRS may be life-threatening or fatal. For better comparability of CRS events, grading of CRS is typically done from 1 (least severe) to 5 (most severe, death).

CRS has been shown to be a key toxicity for bispecific therapy in AML including CD33×CD3 bispecific construct in subjects with R/R AML. For example, too high starting doses as a single dose without prior lower dose-step and/or without premedication can be intolerable due to dose limiting toxicities (DLT) including grade 4 CRS and/or grade 3 colitis. Hence, it is the objective of the present invention to provide an improved administration, i.e. a schedule of administration of the CD33×CD3 bispecific construct to mitigate the risk of CRS while allowing higher CD33×CD3 bispecific construct dosages for better therapeutic outcome in the treatment of AML.

### SUMMARY OF THE INVENTION

In a first aspect, the present invention refers to a bispecific construct comprising a first binding domain specifically binding to CD33 and a second binding domain specifically binding to CD3 preferably for use in a method for the treatment of (i.) myeloid leukemia, selected from relapsed/refractory AML (R/R AML) and AML with minimal residual disease (MRD), or (ii.) myelodysplastic syndrome (MDS), wherein the bispecific construct is administered in one or more treatment cycles, wherein at least one treatment cycle comprises more than 14 days of administration of the bispecific construct in at least three, preferably four or five different dosages applying at least two, preferably three or four dosage steps, optionally followed by a period without administration of the bispecific construct, wherein the bispecific construct is administered in at least one of the one or more treatment cycles according to a schedule comprising the following steps:
(a) administration of a first dosage of the bispecific construct of at least 10 μg per day for use in the treatment of R/R AML or at least 30 μg per day for use in the treatment of MRD or MDS, followed by
(b) administration of a second dosage of the bispecific construct, wherein said second dosage is at least 240 μg per day and/or preferably exceeds said first dose at least 10-fold for use in the treatment of R/R AML or at least 8-fold for use in the treatment of MRD or MDS, and/or wherein the delta between the first and the second dosage is preferably at least 50 μg per day, preferably at least 100, 150, 200, 250, 300, 350, 360 or at most 400 μg per day, followed by
(c) administration of a third dosage of the bispecific construct, wherein said third dosage is at least 600 per day and/or preferably exceeds said second dosage at most three-fold and/or wherein the delta between the second and the third dosage is preferably at least 50 μg per day, preferably at least 100, 150, 200, 250, 300, 350, 360, or at most 400 μg per day for use in the treatment of R/R AML, and wherein said third dosage is in the range of 600 to 1600 μg/d for use in the treatment of MRD or MDS, preferably followed by
(d) administration of a forth dosage of the bispecific construct preferably wherein the bispecific construct is for use in the treatment of R/R AML, wherein said optional forth dose is at least 720 μg per day and/or exceeds said third dosage and/or wherein the delta between the fourth and the fifth dosage is at least 50 μg per day, preferably at least 100, 150, 200, 250, 300, 350, 360 or at most 400 μg per day, optionally followed by
(e) administration of a fifth dosage of the bispecific construct preferably wherein the bispecific construct is for use in the treatment of R/R AML, wherein said optional fifth dose is at least 960 μg per day and/or exceeds said fourth dosage and/or wherein the delta between the fourth and the fifth dosage is at least 50 μg per day, preferably at least 100, 150, 200, 250, 300, 350, 360, or at most 400 μg per day, optionally followed by
(f) administration of a sixth dosage of the bispecific construct preferably wherein the bispecific construct is for use in the treatment of R/R AML, wherein said optional sixth dose is at least 1200 μg per day and/or exceeds said fifth dosage and/or wherein the delta between the firth and the sixth dosage is at least 50 μg per day, preferably at least 100, 150, 200, 250, 300, 350, 360, or at most 400 μg per day.

It is envisaged in one aspect of the present invention that the time of administering the bispecific construct in one treatment cycle including all steps (a) to (c) or (d) or (e) or (f) is at least 15 days, preferably 15 to 60 days, more preferably 28 to 56 days, most preferably 28 days wherein the bispecific construct is for use in the treatment of R/R AML or MRD AML or 56 days wherein the bispecific construct is for use in the treatment MDS.

It is envisaged in one aspect of the present invention that, preferably wherein the bispecific construct is for use in the treatment of R/R AML, the first dosage in step (a) is at least 10 μg per day, preferably in the range of 10 to 20 μg per day, preferably 10 μg per day, the second dosage in step (b) is at least 240 μg per day, preferably in the range of 240 to 600 μg per day, the third dosage in step (c) of at least 600 μg per day, preferably in the range of 600 to 1000 μg per day, and preferably the forth dosage in step (d) of at least 720 μg per day, preferably 720 to 1600 μg per day, more preferably in the range of 960 to 1080 μg per day, more preferably 960 μg per day, optionally the fifth dosage in step (e), of at least 960 μg per day, preferably at least 1200 or 1300 μg per day, and optionally the sixth dosage in step (f), of at least 1200 μg per day, preferably at least 1300 or 1600 μg per day.

It is envisaged in one aspect of the present invention that the period of administration of the first dosage in step (a) is 1 to 5 days, preferably 2 or 3 days, the period of administration of the second dosage in step (b) is 2 to 5 days, preferably 2 or 3 days, and the period of administration of the third dosage in step (c) and the preferred and optional fourth, fifth and sixth dosage in step (d), (e) and (f), respectively, together is 7 to 52 days, preferably 14 to 23 or 52 days, more preferably 22, 23 wherein the use is for the treatment of R/R AML or MRD or 52 days wherein the se is for the treatment of MDS.

It is envisaged in one aspect of the present invention that the treatment of the myeloid leukemia, preferably acute myeloid leukemia, comprises two or more treatment cycles, preferably two, three, four, five, six or seven treatment cycles, whereof at least one, two, three, four five, six or seven treatment cycles comprise more than 14 days of bispecific construct administration.

It is envisaged in one aspect of the present invention that the at least one treatment cycle is followed by a period without administration of the bispecific construct, preferably at least 1, 2, 3, 4, 5, 6, 7, 8, 9, 10, 11, 12, 13 or 14 days without treatment.

It is envisaged in one aspect of the present invention that at least one treatment cycle is not followed by the period without administration of the construct, preferably where the bispecific construct is for use in the treatment of MDS.

It is envisaged in one aspect of the present invention that only the first cycle of the treatment comprises the administration according to step (a), whereas the following cycles start with the dosage according to step (b).

It is envisaged in one aspect of the present invention that the first binding domain of the bispecific construct is a single chain bispecific construct.

It is envisaged in one aspect of the present invention that the first binding domain of the bispecific construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100, preferably 94 to 96 an 98 to 100.

It is envisaged in one aspect of the present invention that the second binding domain of the bispecific construct comprises groups of six CDRs selected from the group consisting of SEQ ID NOs: 148-153, 154-159, 160-165, 166-171, 172-177, 178-183, 184-189, 190-195, 196-201 and 202-207, preferably 202-207.

It is envisaged in one aspect of the present invention that the first binding domain of the bispecific construct comprises groups of six CDRs selected from the group consisting of SEQ ID NOs: 94 to 96 or 98 to 100 and the second binding domain of the bispecific construct comprises groups of six CDRs selected from the group consisting of SEQ ID NOs: 202-207.

It is envisaged in one aspect of the present invention that the first binding domain of the bispecific construct comprises a VH of SEQ ID NO 93 and a VL of SEQ ID NO 97, and wherein the second binding domain of the bispecific construct comprises a VH of SEQ ID NO 208 and a VL of SEQ ID NO 209.

It is envisaged in one aspect of the present invention that the bispecific construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108, preferably selected from the group consisting of SEQ ID NOs: 104, 105, 106, 107 and 108, more preferably SEQ ID NO 104.

It is envisaged in one aspect of the present invention that the bispecific construct is administered in combination with a PD-1 inhibitor, a PDL-1 inhibitor and/or one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the PD-1 inhibitor, a PDL-1 inhibitor and/or one or more epigenetic factors are administered prior to the administration of the bispecific construct;
(b) the PD-1 inhibitor, a PDL-1 inhibitor and/or one or more epigenetic factors are administered subsequent to the administration of the bispecific construct; or
(c) the PD-1 inhibitor, a PDL-1 inhibitor and/or one or more epigenetic factors and the bispecific construct are administered simultaneously.

It is envisaged in one aspect of the present invention that the PD-1 inhibitor, a PDL-1 inhibitor and/or one or more epigenetic factors are administered prior to the administration of the bispecific construct, preferably 1, 2, 3, 4, 5, 6, or 7 days prior to the administration of the bispecific construct.

It is envisaged in one aspect of the present invention that the epigenetic factor is hydroxyurea.

It is envisaged in one aspect of the present invention that the myeloid leukemia is selected from the group consisting of acute myeloblastic leukemia, preferably relapsed or refractory acute myeloid leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and mixed phenotypic acute leukemia.

It is envisaged in another aspect of the present invention that a method for the treatment of myeloid diseases, preferably related one or more of the diseases (i.) myeloid leukemia, selected from relapsed/refractory AML (R/R AML) and AML with minimal residual disease (MRD) AML, or (ii.) myelodysplastic syndrome (MDS) in a patient in need thereof is provided, the method comprising administering a therapeutically efficient amount of a bispecific construct comprising a first binding domain specifically binding to CD33 and a second binding domain specifically binding to CD3 in one or more treatment cycles, wherein the at least one treatment cycle comprises more than 14 days of administration of the bispecific construct in at least three different dosages applying at least two dosage steps,

wherein the bispecific construct is administered in one treatment cycle according to a schedule comprising the following steps:
(a) administration of a first dosage of the bispecific construct of at least 10 μg per day in the treatment of R/R AML or at least 30 μg per day used in the treatment of MRD or MDS, followed by
(b) administration of a second dosage of the bispecific construct, wherein said second dosage is at least 240 μg per day and/or preferably exceeds said first dose at least 10-fold in the treatment of R/R AML or at least 8-fold used in the treatment of MRD or MDS and/or wherein the delta between the first and the second dosage is preferably at least 50 μg per day, preferably at least 100, 150, 200, 250, 300, 350, 360 or at most 400 μg per day, followed by
(c) administration of a third dosage of the bispecific construct, wherein said third dosage is at least 600 per day and/or preferably exceeds said second dosage at most three-fold and/or wherein the delta between the second and the third dosage is preferably at least 50 μg per day, preferably at least 100, 150, 200, 250, 300, 350, 360, or at most 400 μg per day in the treatment of R/R AML, and wherein said third dosage is in the range of 600 to 1600 μg/d used in the treatment of MRD or MDS, preferably followed by
(d) administration of a forth dosage of the bispecific construct preferably wherein the bispecific construct is for use in the treatment of R/R AML, wherein said optional forth dose is at least 720 μg per day and/or exceeds said third dosage and/or wherein the delta between the fourth and the fifth dosage is at least 50 μg per day, preferably at least 100, 150, 200, 250, 300, 350, 360 or at most 400 μg per day, optionally followed by
(e) administration of a fifth dosage of the bispecific construct preferably wherein the bispecific construct is for use in the treatment of R/R AML, wherein said optional fifth dose is at least 960 μg per day and/or exceeds said fourth dosage and/or wherein the delta between the fourth and the fifth dosage is at least 50 μg per day, preferably at least 100, 150, 200, 250, 300, 350, 360, or at most 400 μg per day, optionally followed by
(f) administration of a sixth dosage of the bispecific construct preferably wherein the bispecific construct is for use in the treatment of R/R AML, wherein said optional sixth dose is at least 1200 μg per day and/or exceeds said fifth dosage and/or wherein the delta between the firth and the sixth dosage is at least 50 μg per day, preferably at least 100, 150, 200, 250, 300, 350, 360, or at most 400 μg per day.

It is envisaged in another aspect of the present invention that the time of administering the bispecific construct in one treatment cycle including all steps (a) to (c) or (d) or (e) or (f) is at least 15 days, preferably 15 to 60 days, more preferably 28 to 56 days, more preferred 28 days wherein the bispecific construct is used in the treatment of R/R AML or MRD AML or 56 days wherein the bispecific construct is used in the treatment MDS.

It is envisaged in one aspect of the present invention that preferably wherein the bispecific construct is for use in the treatment of R/R AML the first dosage in step (a) is at least 10 μg per day, preferably in the range of 10 to 20 μg per day, preferably 10 μg per day, the second dosage in step (b) is at least 240 μg per day, preferably in the range of 240 to 600 μg per day, the third dosage in step (c) of at least 600 μg per day, preferably in the range of 600 to 1000 μg per day, and preferably the forth dosage in step (d) of at least 720 μg per day, preferably 720 to 1600 μg per day, more preferably in the range of 960 to 1080 μg per day, more preferably 960 μg per day, optionally the fifth dosage in step (e), of at least 960 μg per day, preferably at least 1200 or 1300 μg per day, and optionally the sixth dosage in step (f), of at least 1200 μg per day, preferably at least 1300 or 1600 μg per day.

It is envisaged in another aspect of the present invention that the period of administration of the first dosage in step (a) is 1 to 5 days, preferably 2 or 3 days, the period of administration of the second dosage in step (b) is 2 to 5 days, preferably 2 or 3 days, and the period of administration of the third and the optional forth dose in step (c) and optional step (d), respectively, is 7 to 52 days, preferably 14 to 23 days, more preferably 21, 22 or 23 days wherein used for the treatment of R/R AML or MRD or 50 or 52 days wherein used for the treatment of MDS.

It is envisaged in another aspect of the present invention that the treatment of the myeloid leukemia comprises two or more treatment cycles, preferably 2, 3, 4, 5, 6 or 7 treatment cycles, whereof at least 1, 2, 3, 4, 5, 6 or 7 treatment cycles which each comprises more than 14 days of bispecific construct administration.

It is envisaged in another aspect of the present invention that the treatment is followed by the period without administration of the bispecific construct, preferably at least 1, 2, 3, 4, 5, 6, 7, 8, 9, 10, 11, 12, 13, or 14 days without treatment.

It is envisaged in another aspect of the present invention that the treatment is not followed by the period of at least 14 days without administration of the bispecific construct, preferably where the bispecific construct is for use in the treatment of MDS, preferably in order to extend exposure which is typically safer in an MDS setting than in an R/R AML setting.

It is envisaged in another aspect of the present invention that only the first cycle of the treatment comprises the administration according to step (a), whereas the following cycles start with the dose according to step (b).

It is envisaged in another aspect of the present invention that the construct is a single chain bispecific construct.

It is envisaged in another aspect of the present invention that the first binding domain of the bispecific construct used in the method of treatment comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100, preferably 94 to 96 an 98 to 100.

It is envisaged in another aspect of the present invention that the second binding domain of the bispecific construct used in the method of treatment comprises groups of six CDRs selected from the group consisting of SEQ ID NOs: 148-153, 154-159, 160-165, 166-171, 172-177, 178-183, 184-189, 190-195, 196-201 and 202-207, preferably 202-207.

It is envisaged in one aspect of the present invention that the first binding domain of the bispecific construct used in the method of treatment comprises groups of six CDRs selected from the group consisting of SEQ ID NOs: 94 to 96 or 98 to 100 and the second binding domain of the bispecific construct comprises groups of six CDRs selected from the group consisting of SEQ ID NOs: 202-207.

It is envisaged in one aspect of the present invention that the first binding domain of the bispecific construct used in the method of treatment comprises a VH of SEQ ID NO 93 and a VL of SEQ ID NO 97, and wherein the second binding domain of the bispecific construct comprises a VH of SEQ ID NO 208 and a VL of SEQ ID NO 209.

It is envisaged in one aspect of the present invention that the bispecific construct is a single chain construct used in the method of treatment comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108, preferably selected from the group consisting of SEQ ID NOs: 104, 105, 106, 107 and 108, more preferably SEQ ID NO 104.

### BRIEF DESCRIPTION OF THE DRAWINGS

FIG. 1: Overview of a Phase I clinical study on CD33×CD3 bispecific construct for use in the treatment of R/R AML comprising 20 patient cohorts. “C” stands for cohort, numbers following the cohort number stand for administered dose levels [μg per day]. One arrow indicates one step to target dose (TD), two arrows indicate to steps to target dose and 3 arrows indicate three steps to target dose. Further abbreviations: ECOG PS, Eastern Cooperative Oncology Group Performance Status; PK, pharmacokinetic; R/R, relapsed/refractory; TD, targeted dose. CR: Complete Remission, CRi: Complete Remission with Incomplete Count Recovery, CRh: Complete Remission with Partial Hematologic Recovery, MLFS: Morphologic Leukemia-Free State.

FIG. 2: Overview of anti-tumor activity with respect to the first 16 patient cohorts in a Phase I clinical study. (A) Best overall response (B) Treatment duration in responders. Anti-tumor efficacy with respect to study cohort:
8 patients responded to CD33×CD3 bispecific construct (as exemplified by SEQ ID NO 104) treatment: CR (n=3, cohorts 11, 15, and 16), CRi (n=4, cohorts 8, 9, 12, and 15) and MLFS (n=1, cohort 2) with 3 responders out of 14 treated patients (21%) in cohorts 15-17. For patients achieving CR/CRi (n=7), minimal efficacious dose was established at the 120 μg/day dose level. Response was observed after 1st cycle and sustained for a median 38.5 days (range of 14-121 days) during the on-study period.

Following CRi, 1 patient treated in cohort 15 was bridged to allogenic hematopoietic stem cell transplant (HSCT) FIG. 3: Correlation of CRS with (A) leukemic burden, (B) E:T (effector-target cell) ratio and (C) Correlation of CRS with IL-10.

FIG. 4: CR/CRi Response to CD33×CD3 bispecific construct (SEQ ID NO: 104), correlation of CR/CRi response with SEQ ID NO: 104 exposures and leukemic burden: (A) SEQ ID NO: 104 exposures, (B) Bone Marrow and (C) peripheral blood.

FIG. 5: Overview on frequency and severity of CRS depended on a schedule (number of dose steps, i.e. one up to four, and dose discrepancies between dose levels, respectively).

FIG. 6: Population PK model parameters and diagnostic plots: Model diagnostics plots

FIG. 7: (A) Baseline tumor burden, (B) Steady-state CD33×CD3 bispecific construct (as exemplified by SEQ ID NO 104) exposures and (C) Baseline E:T ratio were compared for responders vs non-responders to assess whether it influences efficacy of CD33×CD3 bispecific construct (as exemplified by SEQ ID NO 104).

FIG. 8: (A) Baseline tumor burden, (B) Steady-state CD33×CD3 bispecific construct exposures and (C) Baseline CD33 expression on blast cells were plotted against the worst grade CRS on treatment for each patient to explore its influence on safety of CD33×CD3 bispecific construct (as exemplified by SEQ ID NO 104)

FIG. 9: Probability of CRS Grade by CD33×CD3 bispecific construct exposures: Solid line represents mean; dashed line represents 95% CI. Worst grade CRS for each patient was modeled with CD33×CD3 bispecific construct (as exemplified by SEQ ID NO 104) exposures using a proportional odds logistic regression model. The effect of baseline patient characteristics was tested as a covariate in the logistic regression model.

### DETAILED DESCRIPTION OF THE INVENTION

For an efficient treatment of (i.) myeloid leukemia such as AML, preferably R/R AML or minimal residual disease (MRD+) AML and/or (ii.) myelodysplastic syndrome (MDS), using a CD33⁺ cell eliminating therapy approach, safety and tolerability of the employed CD33×CD3 bispecific construct has to be established at dosages which are also clinically effective.

This problem was solved, e.g., by providing a bispecific construct comprising a first binding domain specifically binding to CD33 and a second binding domain specifically binding to CD3 (CD33/CD3) for use in a method for the treatment of (i.) myeloid leukemia or (ii.) MDS, wherein the bispecific construct is administered in one or preferably more treatment cycles, wherein one treatment cycle comprises more than 14 days of administration of the bispecific construct in at least three different, preferably four dosages applying at least two, preferably three dosage steps or even four, five, six, seven, eight, nine or ten dosage steps, optionally followed by a period without administration of the construct. The addressed disease in the context of the present invention is preferably R/R AML, minimal residual disease (MRD+) AML and/or MDS. Advantageously, a CD33×CD3 bispecific construct according to the present invention is suitable for use in the treatment of more than one bone marrow failure syndrome including myeloid leukemia and its typical precursor MDS and can versatilely be used as needed and accordingly dosed as described herein.

Using an administration schedule in line with the present invention, dose steps are intermediate CD33×CD3 bispecific construct dose levels administered with 1-5 day intervals prior to the target dose. Preferred dose steps herein are, for example, at least 10 μg per day in the 1st step, at least 240 μg per day in the 2nd step and at least 600 μg per day in the 3rd step. Typically, a dosage step is at most 400 μg per day higher than the previous dosage step in order to mitigate CRS adverse events in the context of the present invention. A higher number of steps reduces the risk of CRS adverse events in the context of the present invention. Hence, if a target dose, i.e. the highest dose of a dosage regimen which preferably facilitates clinical efficacy with respect to AML, of e.g. 1600 μg per day should be reached, it is safest in line with the present invention, to start with a low dose of 10 μg per day and then perform a series of escalating steps wherein the steps differ in at most 400 μg per day. Further steps might be added in between in line with the present invention, i.e. adding further mini steps in between the at least two, three, four or five steps as disclosed herein in detail. This is particularly useful for use in the treatment of R/R AML where an increased number of steps, such as two or more, typically three, four or even fie steps (leading, in consequence, to three, four, five or even six different ascending dosages) typically and advantageously leads to increased safety in terms of reduced risk of CRS as significant side effect. The same applies in principle also to the use in the treatment of MRD and MDS, where advantageously less steps are required to mitigate CRS as disclosed herein.

According to the present invention, optimized dose steps in the—preferably continuous infusion—administration of a CD33×CD3 bispecific construct allow higher target dose levels with manageable safety profile. In detail, the first step should comprise administration of at least 10 μg per day. Preferably, said dosage should be 10 μg per day or not much above, i.e. below 30 μg per day, because subjects with R/R AML typically have tumor burden ranges from 5% to >50%. Higher tumor burden typically correlates with a higher risk of developing CRS in R/R AML patients. Without wanting to be bound by theory, patients with R/R AML will be at lower risk of developing CRS when treated with a low run in dose of only about 10 μg per day, i.e. 10, 11, 12, 13, 14, 15, 16, 17, 18, 19, 20, 21, 22, 23, 24, 25, 26, 27, 28 or 29 μg per day, preferably 10 μg per day. This typically facilitates the tolerability of an about 10-fold higher second dose, which can be 8, 9, 10, 11, 12, 13, 14, 15, 16, 17, 18, 19, 20, 21, 22, 23, 24, 25, 26, 27, 28, 29 or even 30-times higher than the first dose in order to reach the ultimate target dose quickly, but may typically not exceed the previous dosage by more than 400 μg per day in order to avoid CRS adverse effects. For example, a second dosage of 240 μg per day is preferably tolerated if the first dosage was as low as 10 μg per day. In consequence, a tolerated larger first step then requires only a smaller second step. Hence, in line with the present invention, the third dosage is typically only two-, three, four- or five-fold the second dosage, but preferably only at most three-fold, e.g. at least 600 μg per day or 720 μg per day but might be up to 1080 μg per day. A further optional but preferred third step is again significantly lower than the second step, i.e. typically the fourth dosage is below two-fold or three-fold higher than the third dosage, i.e. typically the third dosage is at least 720 μg per day, 840 μg per day or 960 μg per day but could be as high as about 400 μg per day higher than the second dose to allow for optimal therapeutic efficacy in the treatment of AML, preferably R/R AML. Further steps in between are envisaged in the context of the present invention to reach a higher target dose such at least 1100, 1200, 1300, 1400, 1500 or 1600 μg per day.

Hence, in the context of the present invention a low run in dose followed by a large first step, a second smaller step and a preferred even smaller step, preferably in combination with an immunomodulator such as a cytokine or cytokine receptor blocking agent, e.g. in form of early intervention with tocilizumab, preferably mitigates CRS challenges and still facilitates beneficial clinical outcomes with respect to the treatment of AML, preferably R/R AML. While a minimal efficacious dose of 120 μg per day has been found to reach a clinical effect such as CRi, in order to obtain a more sustainable clinical effect and an improved safety profile at the same time, the administration according to the present invention is more likely to be beneficial for so treated patients. In particular in terms of percentile change from baseline, response is superior for higher dosages applied in more steps as described herein. For example, an administration schedule comprising four subsequent rising dosages, wherein the first is at least 10 μg per day, the second is at least 10-fold the first dosage, e.g. at least 240 μg per day, the third is at most 3-fold the second dosage such as at least 600 μg per day and the fourth exceeds the third dosage, as the previous ones did, by not more than 400 μg per day, e.g. at least 720 or 840 μg per day, then percentile changes from baseline beyond-80%, preferably-90% or even-100% can be achieved. As exemplified herein, such beneficial and surprising results are, e.g., seen in cohorts 15 and 16. Accordingly, applying an administration regimen as described herein, preferably at least 25%, or even at least 30% or at least 50% of the treated patients achieve a complete remission (CR) or complete remission with incomplete hematologic recovery (CRi). At the same time, preferably, only up to 20%, preferably only up to 5% of all treated patients suffer from CRS grade 3 or higher, while not more than up to 50% preferably only up to 40% or 15% require intensive care due to CRS as adverse effect. In view thereof, it is a particular advantage of the present invention to preferably keep overall the rate of CRS grade 2 or higher adverse effects below 50% of all patients treated according to an administration regimen comprising at let three steps, wherein the first step is larger than the second, and the second is larger than the third, and wherein the start (run in) dose is low enough not to initially trigger CRS adverse effects.

For example, in the context of the present invention, it has been observed that an administration schedule according to the present invention such as 10 μg per day as step 1 dose, 240 μg as step 2 dose, 600 μg as step 3 dose and 720 or 840 μg per day as target dose (cohort 16 and 17), wherein the entire schedule administered over 28 days, and preferably given under early intervention by tocilizumab, the CRS adverse effects of moderate grade 2 or higher could be kept at or even below 50% of patient incidence. This is especially remarkable because lower target doses such as 480 μg per day (cohort 14) exhibited Grade 2 or higher CRS patient incidence of 100%. However, there the dosing steps were not adjusted to each other as required by the present invention, i.e. that the first step is significantly larger than the second step and that there is preferably also at least a third step in order to mitigate CRS and provide sufficient clinical efficacy. In this regard, it was observed that frequency and severity of CRS depended on a schedule (number of dose steps) and a Target Dose level. At later cohorts, frequency and severity of CRS may be further mitigated by early use of a cytokine or cytokine receptor blocking agent such as tocilizumab. Also, higher grades of CRS were observed in patients with higher leukemic burden and with higher Effector:Target (E:T) ratio.

Hence, by means of the at least a twofold step dosing with at least three increasing dosage levels, it is possible to preferably efficiently eliminate myeloid leukemic cells during the more than 14 days of CD33/CD3 bispecific construct (e.g. SEQ ID NO: 104) administration period, while still allowing the patient to recover the myeloid compartment in a period without administration of the construct between treatment cycles. Employing a target dosage, i.e. the maximum dosage of the last step within a treatment cycle, of at least 720 μg per day preferably enables a complete remission of the disease as demonstrated herein. At the same time, the step dosing according to the present invention preferably significantly reduces the risk of severe immunologic side effects such as a cytokine release syndrome or symptoms thereof despite longer exposure to the target dose than what has previously been expected to be tolerable. By applying the step dosing according to the present invention, i.e. applying at least two dosage steps resulting in at least three increasing dosages, the patient can be exposed to the target dosage for a prolonged period of time, such as a maximum of 52 days. Said maximum period of time results from the first and the second step lasting for two days, respectively, and the third step lasting 24 days of the remaining first treatment cycle and another 28 days of target dosage of a subsequent (second) treatment cycle which comprises only the third dosage without previous step dosing. Hence, at least one of the treatment-free periods between treatment cycles (i.e. when the bispecific construct according to the present invention is uninterrupted administered) can be dispensable. Accordingly, the target dosage of the first concerned treatment cycle is immediately followed by the same target dosage of the subsequent, i.e. second concerned treatment cycle without interruption. Thereby, exposure of the patient to the target dosage is significantly expanded in order to fulfil the therapeutic goal to eradicate AML blasts and leukemic stem cells as a precondition for long-lasting therapeutic effect and eventually eradication of the AML disease in the affected and so-treated patient. Hence, the method according to the present invention provides a method which balances the need for a preferably long-lasting therapeutic effect, i.e. to eradicate the hematopoietic and the myeloid leukemic stem cells effective, and the avoidance or attenuation of severe and potentially therapy-ending side effects such as CRS. In particular, CRS events of the highest grade 5 (as commonly defined in the art) can be preferably avoided and CRS events of a higher grade 3 and 4 be significantly reduced in occurrence, i.e. grade 3 occurring typically in at most 10% of treated patients and grade 4 typically in at most 5% of treated patients, respectively. In the context of the present invention, the duration of exposure of a patient to the bispecific construct in one treatment cycle is longer than 14 days and can be up to 60 days, if two treatment cycles are not separated by a treatment-free period. Typically, each treatment cycle comprising at least two, preferably three dosage steps is followed by a treatment-free period to allow for patient recovery. However, when prolong target dosage exposure is required to address leukemic stem cells in addition to AML blasts, two treatment cycles are connected to each other by leaving the treatment-free period away. However, preferably not more than two treatment cycles follow each other without a treatment-free period in order to allow for sufficient patient recovery but still prolong target dosage exposure time.

When said two treatment cycles are connected, the later treatment cycle following the earlier treatment cycle is characterized by having only one dosage and no step dosing. This is facilitated by the fact that the step dosing of the earlier treatment cycle reduces the risk for side effects such as CRS (especially of higher grades 3 and 4 and highest grade 5) also for the immediately following treatment cycle (i.e. with no treatment-free period between the two connected cycles) because the treatment cycle following the earlier treatment cycle profits from the earlier treatment cycle's applied step dosing. Hence, side effect CRS of the highest grade could be avoided completely and the higher grades 3 and 4 attenuated to infrequent single digit occurrences. A treatment interruption could be avoided in the majority of treated patients and ensure continuous effective dose administration to treat high patients suffering from highly progressive r/r AML.

Hence, in the context of the present invention, at least one of the treatment cycles has to fulfil the requirements for the specific step dosing as described herein. In case only one treatment cycle is applied, said one treatment cycle comprises the step dosing. In case two treatment cycles are applied which are not separated by a treatment-free period, then it is sufficient for only the first of the two treatment cycles to fulfil the requirements of the specific at least three-step specific step dosing as described herein.

The period of exposure as referred to herein typically refers to the total exposure to all at least three different dosages applied through one treatment cycle. Typical exposure to the target dose is shorter, i.e. shortened by the duration of the first and second (and optionally third) dosage before the third or optional forth maximum (target) dosage within the treatment cycle is reached. Such exposure of the target dosage may last for, for example, 56, 55, 54, 53, 52, 51, 50, 25, 24, 23, 22, 21, 20, 19, 18, 17, 16, 15 or 14 days, which at the same time allows for full exploitation if the anti-tumor efficacy of the CD33×CD3 bispecific construct (e.g. SEQ ID NO: 104) according to the present invention. Consequently, the present dosage regimen allows for a prolonged exposure of the treated patient to the target dose while minimizing the side effects during the initial phase of drug administration, such as cytokine release syndrome and symptoms thereof, by using step dosing as described herein. At the same time, the superior efficacy, which is confined by the administration schedule or dosage regimen as described herein, is preferably demonstrated by a significant reduction in tumor burden in treated patients, more preferably in partial or even complete remission or even repeated complete remissions after one treatment cycle or a plurality of treatment cycles, respectively.

A typical treatment cycle according to the present invention, which has clinically demonstrated complete remission of disease (AML) comprises administering the CD33×CD3 bispecific construct (e.g. SEQ ID NO: 104) a first dosage of 10 μg per day for two or three consecutive days, immediately followed by a second dosage of 60 μg per day for 2, 3 or 4 days, immediately followed by a third dosage of 240 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 μg per day for 2, 3 or 4 days, immediately followed by a third dosage of 480 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 μg per day for 2, 3 or 4 days, immediately followed by a third dosage of 600 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60, 120 or 240 μg per day for 2, 3 or 4 days, immediately followed by a third dosage of 720 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60, 120 or 240 μg per day for 2, 3 or 4 days, immediately followed by a third dosage of 840 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 840 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 960 μg per day for 21, 22 or 23 days, wherein the total treatment cycle duration is 28 days. Such treatment cycles are also represented in FIG. 5 for better illustration.

It has been a remarkable finding in the context of the present invention that already a target dosage of 240 μg per day can lead to complete remissions of disease AML, being MRD+ but preferably also MRD−. Higher target dosages such as described herein, e.g. from 600 μg per day for the MRD+ AML indication or e.g. from 720 μg per day for the R/R AML indication, do typically even more quantitatively eradicate leukemic stem cells in addition to AML blasts and likely reduce the risk of relapse and thus, provide a longer disease-free state for the patient, improving their quality of life.

Preferably, in the context of the present invention, the dose toxicity limiting (DLT) window can be shortened to a standard of 4 weeks (with at least 14 days on the target dose) allowing for monitoring the onset of CRS and its resolutions, efficient intra-subject escalation, and overall patient safety. As it is known in the art, the expression of CD33 on the surface of myeloid cells comprising the common myeloid progenitor cells, Myeloblasts, Monocytes has been demonstrated in the literature by flow cytometry. Moreover, CD33 expression on the surface of Macrophages has been demonstrated via immunohistochemistry.

For a successful treatment of a myeloid leukemia a significant exposure of a patient (i.e. a certain length of exposure) with the bispecific constructs described herein is required to induce T cell activation/proliferation and cytotoxic activity of those T cells. However, based on the above described observations the longer the administration period of the bispecific constructs last, the longer pancytopenia is to be expected. This in mind, the solution to the problem underlying this invention is to balance the length of exposure and the dose of the bispecific constructs which enable the effective elimination of the leukemic cells with an off treatment period during which the myeloid compartment of a patient is allowed to recover. This is reflected by the above described administration scheme.

The time period without administration serves as recovery period for the myeloid compartment in order to rebuild myeloid cells important, e.g., for the defense against bacterial infection. The length of the required minimum time period without administration typically depends on the residual tumor burden. For example, patients who have shown a partial response, the time period may be as short as 7 days or less, such as 1, 2, 3, 4, 5, or 6 days, preferably 7 days, while those patients with higher residual tumor burden and more damage to the myeloid compartment typically require a longer period to rebuild myeloid cells, typically at least 8, 9, 10, 11, 12, 13 or 14 days, preferably 14 days. In general, it is envisaged that exposure of the patient to the target dose is maximized, and at the same time to limit the duration of a single treatment cycle including the treatment free recovery period as much as possible to allow for overall quick sequence of treatment cycles for patients who often are in a critical condition and typically need quick efficacy.

In a particular embodiment of the present invention, a first treatment cycle comprising an administration time of more than 14 days, offers a longer exposure of the patient to the target dose and thereby reduces the tumor burden to such a level that subsequent treatment cycles may not require administration times of more than 14 days. In such a case, treatment cycles after the first treatment cycle may last at most 14 days which reduces the risk of side effects by longer treatment to recovery time ratio within one cycle, provided sufficient efficiency has been reached. Alternatively, the second, third, fourth or any subsequent treatment cycle may last more than 14 days followed by one or more treatment cycles of at most 14 days in length. Also, treatment cycles of more than 14 days of administration may alternate with treatment cycles of at most 14 days of administration in order to level efficacy and mitigation of side effects.

It is a specific achievement of the invention as described herein to provide a dosage scheme which does not loose time to reach an effective dose to target cancer cells and at the same time reduces the risk of triggering severe side effects such as CRS. Wasting time would not be in the interest of the treated patient who is suffering from a severe and aggressively progredient disease. On the other hand, triggering side effects such as CRS by too quick step dosing likely leads to the interruption or abandonment of treatment due to undue toxicity. Both disadvantages are mitigated by the method according to the present invention. Also, by adding a forth step, the delta between the dosages is reduced whereby also the probability of CRS is reduced. Hence, in the context of the present invention, where a high target dosage of, for example, at least 480, 720 or 960 μg per day is applied, a step dosing comprising four steps (e.g. 30-240-600-900 μg per day) is be preferred to a step dosing comprising three steps (e.g. 30-240-900 μg per day), even if conducted over the same time period due to a smaller delta between the dosage of a previous step and a target dosage.

The method according to the present invention avoids or attenuates severe side effects such as CRS. In particular, CRS events of the highest grade 5 (as commonly defined in the art) can be preferably avoided and CRS events of a higher grade 3 and 4 be significantly reduced in occurrence, i.e. grade 3 occurring typically in at most 10% of treated patients and grade 4 typically in at most 5% of treated patients undergoing a method as described herein, respectively.

As the person skilled in the art is aware of, after each relapse, a new complete remission is more and more difficult to achieve. Given the fact that patients undergoing presently described therapy by the bispecific construct have typically already undergone standard chemotherapy and might have gone through remissions and relapses, a profound activity needs to be conferred by the method according to the present invention. Hence, prolonged exposure to a high dosage of bispecific construct, e.g. SEQ ID NO 104, is preferred as described herein. This typically requires a step dosing comprising three dosing steps meaning 4 different and ascending dosages, i.e. a first, second, third and fourth dose. Hence, such a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 840 μg per day for 21, 22 or 23 days, wherein the total treatment cycle duration is 28 days. Alternatively, a preferred treatment cycle comprises a first dosage of 10 μg per day for two or three consecutive days, immediately followed by a second dosage of 60 or 120 μg and a third dosage of 120 or 240 μg per day for together 2 days, immediately followed by a forth dosage of 960 μg per day for 21, 22 or 23 days, wherein the total treatment cycle duration is 28 days. When two treatment cycles are combined and following each other without an intermittent administration-free period, the application of the forth, effective dosage has a duration of up to 52 days. Suh parameters are, for example, considered a prolonged exposure to a high dosage of bispecific construct, e.g. SEQ ID NO 104, is preferred as described herein.

It is envisaged in the context of the present invention that a significant unmet medical need exists for patients with MRD+ AML who are ineligible for HSCT, as there are no approved treatment options for this population. A CD33×CD3 bispecific construct dosed according to the present invention is advantageously effective in treating patients with MRD+ AML which are converted MRD+ to MRD− status, which may improve survival outcomes. Treating subjects who achieved CR with complete hematologic recovery allow to assess the effect of the CD33×CD3 bispecific construct such as SEQ ID NO: 104 on normal myeloid cells and describe any potential changes including the onset, severity, and duration of myelosuppression. Treating subjects who achieved CRi allow to assess the effect of the CD33×CD3 bispecific construct treatment on the recovery of normal myeloid cells.

MDS as defined by the WHO classification, (patients with intermediate, high and very high risk MDS per IPSS-R), who are refractory to hypomethylating agents (HMAs), and who are not eligible for allogenic HSCT (per investigator assessment, lack of a donor, or declined the offered procedure). As discussed in Section 2.1.2 of Appendix 1, a significant unmet medical need exists for patients with MDS who failed HMA treatment and are ineligible for HSCT. Although HMAs are considered the standard of care treatment for MDS, only half of patients respond to this treatment. Moreover, all patients will eventually become refractory to HMAs (Gil Perez and Montalban Bravo, 2019). Patients who failed HMA treatment have a poor prognosis and limited therapeutic options, as there are no approved interventions for HMA refractory MDS (Montalban-Bravo and Garcia-Manero, 2018). Although allogenic HSCT is potentially curative, it is typically only available to younger, fit patients due to the high risk of HSCT associated morbidity and mortality. The Center for International Bone Marrow Transplant Registry (CIBMTR) reported approximately 1150 transplants for MDS and myeloproliferative neoplasms in the US in 2017 (D'Souza and Fretham, 2018). When considered in the context of the entire patient population of high-risk MDS patients in the US, statistics suggest a low use of transplantation in this population, with a cross-sectional survey and prospective feasibility study reporting 4% to 5% of MDS patients receiving transplants (Sekeres et al, 2008; Estey et al, 2007). Therefore, novel therapeutic options are needed for patients with MDS who have progressive or refractory disease following treatment with HMAs and who are ineligible for allogenic HSCT. Because CD33 is expressed on both MDS blasts and myeloid derived suppressor cells (Section 2.2 of Appendix 1), AMG 330 will be evaluated for the treatment of patients with MDS. The baseline tumor burdens in patients with either MRD+ AML or MDS (typically about <5% and <20% blasts in the bone marrow, respectively) may differ and are typically lower compared with the higher tumor burdens found in those with R/R AML (range, 5% to >50% blasts in the bone marrow). Therefore, a lower incidence and severity of CRS is typically found in the MRD+ AML and MDS populations after treatment with a CD33×CD3 bispecific construct according to the present invention compared with that observed in subjects with R/R AML. As such, these three patient populations will each require a step dose schedule according to the present invention with specifically preferred start and final target dose. Alternatively, subjects with either MDS or MRD+ AML will require fewer dose steps and a higher target dose compared with subjects with R/R AML.

While a starting step dose of CD33×CD3 bispecific construct (e.g. SEQ ID NO: 104) of typically at least 10 μg is being used for subjects with a myeloid leukemia, e.g. R/R AML, or a precursor disease thereof, a continuously infused dose of at least 10 μg per day can be further specified, i.e. increased, for use in the treatment of MRD+ AML for best efficacy while maintaining safety as described herein. CD33×CD3 bispecific constructs of the present invention (e.g. SEQ ID NO: 104) typically have steady-state concentrations (Css) of 1.5 to 2 ng/mL (˜240 μg/day) which are considered both safe and efficacious, as these exposures are 4 to 5 times lower than those observed at the highest nonseverely toxic dose (HNSTD) of 10 μg/kg/day in healthy monkeys (average Css=8.36 ng/mL) and above the human dose with 50% tumor growth inhibition (ED50; based on a PK-tumor dynamic model) of 1.44 ng/mL. Hence, it has been established herein for the first time that at least 30 μg per day as a dose level can be used as an advantageous starting dose. Furthermore, a dose of 240 μg/day is supported by the clinical safety and efficacy experience of CD33×CD3 bispecific construct in the R/R AML population, as this dose was well tolerated, with no cases of grade greater of equal to 3 CRS (0 of 15 subjects) and a 20% rate of CR with A CD33×CD3 bispecific construct dose of about 240 μg/day might lead approximately to a 28% risk of developing grade greater or equal to 2 CRS in patients with R/R AML who have a <20% baseline tumor burden. Because patients with MRD+ AML have a baseline tumor burden <5%, the risk of CRS is expected to be even lower in this population than in the R/R AML and MDS populations, which have 5% to >50% and <20% blasts at baseline, respectively. Thus, an at least 240 μg dose level can used as a second dose step prior to the target dose. Because the safety of the at least 600 μg/day target dose was already shown in R/R AML (typically at least 10 μg/day for 2 days, at least 240 μg/day for up to 5 days, and at least 600 μg/day starting at day 8 in a, for example, 28 day treatment cycle) as shown herein, a starting target dose of at least 600 μg/day is found tolerable and effective in the context of the present invention. Preferably, the target dose of at least 600 μg/d is, for example, at least 720, at least 840, at least 960, at least 1080 μg/d, at least 1300 pd/d, or at least 1600 μg/d.

Surprisingly, already applying a two-step dosage regimen of CD33×CD3 bispecific construct (as exemplified herein by SEQ ID NO 104), wherein one treatment cycle comprises at least 28 days, the cycle comprising three different dosages of at least 30 μ/d as initial dose followed by a dose of at least 240 μg/d followed of a target dose of at least 600 μg/d may effectively convert an AML patient of MRD+ status to MRD− status and, thus, reduce the patients risk of a future disease progression. It is a further particular advantage that a low number of three different dosages, i.e. to dosage steps, is typically sufficient to reach the target dose for use in the treatment of MRD AML. A lower number of steps may reduce the level of complexity of treatment and may further increase patient compliance. Also, with fewer number of steps, a fewer number of infusions of premedication, i.e. typically dexamethasone, are required as CRS prophylaxis. Since the premedication typically is an immunosuppressant, it may potentially reduce efficacy of the treatment. Further advantageously, the bispecific construct for use in the treatment of MRD AML involving a step dosing as described herein typically leads to a lower risk in MRD patients to develop severe side effects such as higher degree CRS than in a comparable R/R setting. Typically, CRS is observed of grade 2 or lower, preferably at most grade 1 or lower under a two-step dosage regimen of CD33×CD3 bispecific construct (as exemplified herein by SEQ ID NO 104). Hence, the present bispecific construct is particularly preferred for use in the treatment of MRD AML, even more preferred in a dosage regimen as described herein.

For use of a CD33×CD3 bispecific construct in the treatment of MDS, the preferred starting dose of at least 10 μg/d is preferably at least 30 μg/day. Because patients with MDS have a lower tumor burden than those with R/R AML, patients with MDS are found to be at lower risk of developing CRS than those with R/R AML. As such, subjects with MDS are found to typically tolerate a higher starting dose of the CD33×CD3 bispecific construct (e.g. SEQ ID NO: 104), typically allow a dosing schedule which does not exceed the minimum amount of at least three or at least four steps, i.e. may typically require fewer step doses, and a higher MTD than for use in the treatment of R/R AML. In the context of the present in invention, the for use in treatment of MDS typically comprises at least the dosing steps of at least 10 μg/d, preferably at least 30 μg/day for at least 1 day or at least 2 days, then at least 240 μg/day for up to 5 days, and then at least 600 μg/d, preferably at least 720, 840, 960, 1080, 1300 or 1600 μg/day as target dose for up to 21 days per cycle, wherein typically at least one cycle is performed.

The end of the period of administration is understood to be reached, when the serum level of the active compound, e.g. the bispecific compound drops under a defined threshold. An example for such threshold is a serum level below an EC₉₀ value, preferably below an EC₅₀ value, more preferably below an EC₁₀ value. Such EC values can be defined in a cytotoxic assay using CD33+ target cells and human PBL as effector cells in line with the assays.

In case of a bispecific single chain construct such as a preferred CD33×CD3 bispecific construct in the context of the present invention (see SEQ ID NO: 104), which is known to have a short serum half-life the half-life of CD33×CD3 bispecific construct in mice is 6.5 to 8.7h, while the predicted half-life of CD33×CD3 bispecific construct in human is about 2 hours), the serum level would fall below the above discussed threshold value within short time after stopping a continuous iv administration, i.e. almost immediately after the end of the administration phase.

An assay for the determination of a specific ECₓ value of a bispecific construct suitable for the present invention is described in the examples herein below.

The term “dose” is understood herein as a measured quantity of the agents described herein, i.e. a bispecific construct, typically in units of mass such as microgram [μg].

The term “dosage” is understood herein as the rate of application of a dose of the agents described herein, i.e. a bispecific construct, typically in units of mass per time such as microgram per day [μg/d]. In the context of the present invention, the application is IV infusion, preferably continuous IV infusion (CIV). Therein, administration, i.e. submission of the therapeutic bispecific construct, is not interrupted during the provided period of administration.

The term “treatment cycle” is understood herein as a period of treatment, comprising at least two dosage steps resulting in at least three dosages to be applied, wherein the dosages are increasing by order of their sequence. Said dosages within one treatment cycle are preferably not interrupted by any treatment-free period between the different dosages administered within one treatment cycle applying step dosing as described herein. Instead, the continuous infusion continues with respect to the treated patient preferably uninterrupted for the entire length of the treatment cycle. After competition, said treatment cycle may then typically be followed by a period of rest (administration-free period, i.e. no treatment), and that combination of treatment period and treatment-free period is repeated on a regular schedule.

For example, treatment given for four weeks followed by two weeks of rest is one treatment cycle. When this cycle is repeated multiple times on a regular schedule, it makes up a course of treatment.

The term “step dosing” is understood herein as the application of a series of increasing dosages, preferably within one treatment cycle, in order to avoid treatment-associated side effects such as CRS.

The term “dosage step” is understood herein as the change from one dosage to another. Hence, if the step dosing provides three different dosages, two dosage steps have to be applied, i.e. the change from the first to the second dosage step and from the second to the third dosage step, respectively.

In the context of the present invention, remission is understood either as the reduction or disappearance of the signs and symptoms of the disease AML. The term may also be used to refer to the period during which this diminution occurs. Herein, a remission may be considered a partial remission or a complete remission. For example, a partial remission for AML may be defined as a 50% or greater reduction in the measurable parameters of AML as may be found, for example, on physical examination, radiologic study, or by biomarker levels from a blood or urine test.

In the context of the present invention, complete remission, is typically a total disappearance of the manifestations of a disease. A patient whose condition is in complete remission might be considered cured or recovered, notwithstanding the possibility of a relapse, i.e. the reappearance of a disease.

In the context of the present invention, complete remission (CR) without a number typically means a first CR e.g. a newly diagnosed patient with AML receives chemotherapy in one or more cycles—i.e. before receiving a bispecific construct according to the present invention, and goes into remission, that is the first CR (usually only called CR), then relapses, receives some other therapy and goes into remission again, that is now the second complete remission (CR2) and so forth.

The term “cohort” is understood in the context of the present invention as a group of patients who share a defining characteristic, i.e. who undergo the same treatment cycles characterized by same step dosing, dosages and application duration.

The term “effective dosage” is the target dose of at which the AML blasts and leukemic stem cells are effectively killed. This dose is typically the highest and preferably last dosage of one treatment cycle.

The term “Acute myeloid leukemia” (AML) is understood herein as a common form of acute leukemia, with a rising incidence attributed to an aging population, an increase in environmental exposure, and an increase in the population of cancer survivors previously exposed to chemotherapy and therapeutic radiation.

The term “measurable residual disease” or equivalently “minimal residual disease” (MRD) is understood herein as the persistence of detectable leukemic blasts below the threshold for CR. A patient under such condition may designated as “MRD+”. MRD is a proven risk factor for relapse and poor survival in patients with acute lymphoblastic leukemia (ALL), and the concept of MRD is gaining wider acceptance in the AML community (Richard-Carpentier et al, 2019; Buccisano et al, 2018). Hence, it is an object of the present invention to provide a CD33×CD3 bispecific construct—and its specific dosage regimen—for use in the treatment of MRD+ AML patients. The preferred endpoint of such use for the treatment of AML is the conversion of an AML patient from MRD+ status to MRD− status which is typically characterized by the absence of detectable leukemic blasts. Such abnormal blasts are referred herein simply as blasts if nothing else is mentioned.

The term “myelodysplastic syndrome represents” (MDS) is understood herein as a common class of acquired bone marrow failure syndromes, primarily in adults, and is typically defined by a diverse group of clonal disorders of hematopoietic progenitor cells. MDS is typically characterized by cytopenias, abnormal cell morphology, and progression to AML, typically with up to 30% of MDS cases progressing to AML (Greenberg et al, 1997). Approximately 40 000 new cases of MDS occur annually, with an estimated prevalence of 60 000 to 120 000 cases in the US (Bejar and Steensma, 2014).

The term “bispecific construct” refers to a molecule having a structure suitable for the specific binding of two individual target structures. In the context of the present invention such bispecific constructs specifically bind to a target, preferably CD33 on the cell surface of target cells and an effector, preferably CD3 on the cell surface of T cells. However, the preferred administration as described herein, i.e. a step dosing to mitigate side effects such as a cytokine release syndrome, and a prolonged exposition to maximize efficacy, applies also to other bispecific constructs targeting another target than CD33 in addition to CD3 on the cell surface of T cells. In a preferred embodiment of a bispecific construct at least one, more preferably both binding domains of the bispecific construct are is/are based on the structure and/or function of an antibody. Such constructs may be designated as “bispecific constructs” in line with the present invention.

The term “construct” refers to a molecule in which the structure and/or function is/are based on the structure and/or function of an antibody, e.g., of a full-length or whole immunoglobulin molecule. A construct is hence capable of binding to its specific target or antigen and/or is/are drawn from the variable heavy chain (VH) and/or variable light chain (VL) domains of an antibody or fragment thereof. Furthermore, the domain which binds to its binding partner according to the present invention is understood herein as a binding domain of a construct according to the invention. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to an specific antibody competing with the epitope of the defined antibody. The antibodies on which the constructs according to the invention are based include for example monoclonal, recombinant, chimeric, deimmunized, humanized and human antibodies.

The binding domain of an construct according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of constructs according to the invention are e.g. described in WO 00/006605, WO 2005/040220, WO 2008/119567, WO 2010/037838, WO 2013/026837, WO 2013/026833, US 2014/0308285, US 2014/0302037, W 02014/144722, WO 2014/151910, and WO 2015/048272.

Furthermore, the definition of the term “constructs” includes monovalent, bivalent and polyvalent/multivalent constructs and, thus, monospecific constructs, specifically binding to only one antigenic structure, as well as bispecific and polyspecific/multispecific constructs, which specifically bind more than one antigenic structure, e.g. two, three or more, through distinct binding domains. Moreover, the definition of the term “constructs” includes molecules consisting of only one polypeptide chain as well as molecules consisting of more than one polypeptide chain, which chains can be either identical (homodimers, homotrimers or homooligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The constructs of the present invention are preferably “in vitro generated constructs”. This term refers to an construct according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

An embodiment of the bispecific construct of the present invention is a “single chain constructs”. Those single chain constructs include only above described embodiments of constructs, which consist of a single peptide chain.

The term “monoclonal antibody” (mAb) or monoclonal construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore system can be used to increase the efficiency of phage antibodies which bind to an epitope of a target antigen, such as the target cell surface antigen CD33 or CD3 epsilon (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO96/33735.

A monoclonal antibody can also be obtained from a non-human animal, and then modified, e.g., humanized, deimmunized, rendered chimeric etc., using recombinant DNA techniques known in the art. Examples of modified constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the constructs involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human the target cell surface antigen CD33. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and constructs of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, construct or antibody fragment may also be modified by specific deletion of human T cell epitopes (a method called “deimmunization”) by the methods disclosed in WO 98/52976 and WO00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. MoI. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, constructs or fragments thereof (such as Fv, Fab, Fab′, F(ab′)₂ or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400.

The term “human antibody”, “human construct” and “human binding domain” includes antibodies, constructs and binding domains having antibody regions such as variable and constant regions or domains which correspond substantially to human germline immunoglobulin sequences known in the art, including, for example, those described by Kabat et al. (1991) (loc. cit.). The human antibodies, constructs or binding domains of the invention may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies, constructs or binding domains can have at least one, two, three, four, five, or more positions replaced with an amino acid residue that is not encoded by the human germline immunoglobulin sequence. The definition of human antibodies, constructs and binding domains as used herein also contemplates fully human antibodies, which include only non-artificially and/or genetically altered human sequences of antibodies as those can be derived by using technologies or systems such as the Xenomouse.

In some embodiments, the constructs of the invention are “isolated” or “substantially pure” constructs. “Isolated” or “substantially pure” when used to describe the construct disclosed herein means an construct that has been identified, separated and/or recovered from a component of its production environment. Preferably, the construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The constructs may e.g constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an construct in a wide variety of organisms and/or host cells that are known in the art. In preferred embodiments, the construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated construct will be prepared by at least one purification step.

The term “binding domain” characterizes in connection with the present invention a domain which (specifically) binds to/interacts with/recognizes a given target epitope or a given target site on the target molecules (antigens) and CD3, respectively. The structure and function of the first binding domain (recognizing the target cell surface antigen CD33), and preferably also the structure and/or function of the second binding domain (CD3), is/are based on the structure and/or function of an antibody, e.g. of a full-length or whole immunoglobulin molecule. According to the invention, the first binding domain is characterized by the presence of three light chain CDRs (i.e. CDR1, CDR2 and CDR3 of the VL region) and three heavy chain CDRs (i.e. CDR1, CDR2 and CDR3 of the VH region). The second binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are preferably in the form of polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids). The term “polypeptide” as used herein describes a group of molecules, which usually consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Antibodies and constructs comprising at least one human binding domain avoid some of the problems associated with antibodies or constructs that possess non-human such as rodent (e.g. murine, rat, hamster or rabbit) variable and/or constant regions. The presence of such rodent derived proteins can lead to the rapid clearance of the antibodies or constructs or can lead to the generation of an immune response against the antibody or construct by a patient. In order to avoid the use of rodent derived antibodies or constructs, human or fully human antibodies/constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or constructs are expected to minimize the immunogenic and allergic responses intrinsic to mouse or mouse-derivatized mAbs and thus to increase the efficacy and safety of the administered antibodies/constructs. The use of fully human antibodies or constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide constructs comprising a fully human binding domain against the target cell surface antigen and a fully human binding domain against CD3 in order to vitiate concerns and/or effects of HAMA or HACA response.

The terms “(specifically) binds to”, (specifically) recognizes”, “is (specifically) directed to”, and “(specifically) reacts with” mean in accordance with this invention that a binding domain interacts or specifically interacts with one or more, preferably at least two, more preferably at least three and most preferably at least four amino acids of an epitope located on the target protein or antigen (the target cell surface antigen CD33/CD3).

The term “epitope” refers to the site on an antigen to which a binding domain, such as an antibody or immunoglobulin or derivative or fragment of an antibody or of an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”. The term “epitope” is understood in connection with this application as describing the complete antigenic structure, whereas the term “part of the epitope” may be used to describe one or more subgroups of the specific epitope of a given binding domain.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically a conformational epitope comprises an increased number of amino acids relative to a linear epitope. With regard to recognition of conformational epitopes, the binding domain recognizes a three-dimensional structure of the antigen, preferably a peptide or protein or fragment thereof (in the context of the present invention, the antigen for one of the binding domains is comprised within the target cell surface antigen CD33). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

The interaction between the binding domain and the epitope or epitope cluster implies that a binding domain exhibits appreciable affinity for the epitope or epitope cluster on a particular protein or antigen (here: the target cell surface antigen CD33 and CD3, respectively) and, generally, does not exhibit significant reactivity with proteins or antigens other than the target cell surface antigen CD33 or CD3. “Appreciable affinity” includes binding with an affinity of about 10−6 M (KD) or stronger. Preferably, binding is considered specific when the binding affinity is about 10⁻¹² to 10⁻⁸ M, 10⁻¹² to 10⁻⁹M, 10⁻¹² to 10⁻¹⁰M, 10⁻¹¹ to 10⁻⁸M, preferably of about 10⁻¹¹ to 10⁻⁸M. Whether a binding domain specifically reacts with or binds to a target can be tested readily by, inter alia, comparing the reaction of said binding domain with a target protein or antigen with the reaction of said binding domain with proteins or antigens other than the target cell surface antigen CD33 or CD3. Preferably, a binding domain of the invention does not essentially or substantially bind to proteins or antigens other than the target cell surface antigen CD33 or CD3 (i.e., the first binding domain is not capable of binding to proteins other than the target cell surface antigen CD33 and the second binding domain is not capable of binding to proteins other than CD3).

The term “does not essentially/substantially bind” or “is not capable of binding” means that a binding domain of the present invention does not bind a protein or antigen other than the target cell surface antigen CD33 or CD3, i.e., does not show reactivity of more than 30%, preferably not more than 20%, more preferably not more than 10%, particularly preferably not more than 9%, 8%, 7%, 6% or 5% with proteins or antigens other than the target cell surface antigen CD33 or CD3, whereby binding to the target cell surface antigen CD33 or CD3, respectively, is set to be 100%.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-site with its specific antigen may result in a simple binding of said site to the antigen. Moreover, the specific interaction of the antigen-interaction-site with its specific antigen may alternatively or additionally result in the initiation of a signal, e.g. due to the induction of a change of the conformation of the antigen, an oligomerization of the antigen, etc.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms a single antigen-binding site.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM) and provide a scaffold for the six CDRs in three dimensional space to form an antigen-binding surface. The variable domains of naturally occurring heavy and light chains each comprise four FRM regions (FR1, FR2, FR3, and FR4), largely adopting a f-sheet configuration, connected by three hypervariable regions, which form loops connecting, and in some cases forming part of, the f-sheet structure. The hypervariable regions in each chain are held together in close proximity by the FRM and, with the hypervariable regions from the other chain, contribute to the formation of the antigen-binding site (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. MoI. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. MoI. Biol., 1987, 196: 901; Chothia et al., Nature, 1989, 342: 877; Martin and Thornton, J. MoI. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding site. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, C A, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection.

The term “bispecific” as used herein refers to a construct which is “at least bispecific”, i.e., it comprises at least a first binding domain and a second binding domain, wherein the first binding domain binds to one antigen or target, and the second binding domain binds to another antigen or target (here: CD3). Accordingly, bispecific constructs according to the invention comprise specificities for at least two different antigens or targets. The term “bispecific construct” of the invention also encompasses multispecific constructs such as trispecific constructs, the latter ones including three binding domains, or constructs having more than three (e.g. four, five . . . ) specificities. In case the construct used in connection with this invention is an construct, these encompassed corresponding constructs are multispecific constructs such as trispecific constructs, the latter ones including three binding domains, or constructs having more than three (e.g. four, five . . . ) specificities.

Given that the constructs according to the invention are (at least) bispecific, they do not occur naturally and they are markedly different from naturally occurring products. A “bispecific” construct or immunoglobulin is hence an artificial hybrid antibody or immunoglobulin having at least two distinct binding sites with different specificities. Bispecific antibodies can be produced by a variety of methods including fusion of hybridomas or linking of Fab′ fragments. See, e.g., Songsivilai & Lachmann, Clin. Exp. Immunol. 79:315-321 (1990).

The at least two binding domains and the variable domains of the construct of the present invention may or may not comprise peptide linkers (spacer peptides). The term “peptide linker” defines in accordance with the present invention an amino acid sequence by which the amino acid sequences of one (variable and/or binding) domain and another (variable and/or binding) domain of the construct of the invention are linked with each other. An essential technical feature of such peptide linker is that said peptide linker does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the construct of the invention.

In the event that a linker is used, this linker is preferably of a length and sequence sufficient to ensure that each of the first and second domains can, independently from one another, retain their differential binding specificities. For peptide linkers which connect the at least two binding domains in the construct of the invention (or two variable domains), those peptide linkers are preferred which comprise only a few number of amino acid residues, e.g. 12 amino acid residues or less. Thus, peptide linker of 12, 11, 10, 9, 8, 7, 6 or 5 amino acid residues are preferred. An envisaged peptide linker with less than 5 amino acids comprises 4, 3, 2 or one amino acid(s) wherein Gly-rich linkers are preferred. A particularly preferred “single” amino acid in context of said “peptide linker” is Gly. Accordingly, said peptide linker may consist of the single amino acid Gly. Another preferred embodiment of a peptide linker is characterized by the amino acid sequence Gly-Gly-Gly-Gly-Ser, i.e. Gly₄Ser, or polymers thereof, i.e. (Gly₄Ser)x, where x is an integer of 1 or greater. The characteristics of said peptide linker, which comprise the absence of the promotion of secondary structures are known in the art and are described e.g. in Dall′Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers which also do not promote any secondary structures are preferred. The linkage of said domains to each other can be provided by, e.g. genetic engineering, as described in the examples. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001).

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dubel (2010), loc. cit. and Little (2009), loc. cit.) can be adapted to produce single chain constructs specifically recognizing (an) elected target(s).

Bivalent (also called divalent) or bispecific single-chain variable fragments (bi-scFvs or di-scFvs having the format (scFv)₂) can be engineered by linking two scFv molecules. In case these two scFv molecules have the same binding specificity, the resulting (scFv)₂ molecule will preferably be called bivalent (i.e. it has two valences for the same target epitope). In case the two scFv molecules have different binding specificities, the resulting (scFv)₂ molecule will preferably be called bispecific. The linking can be done by producing a single peptide chain with two VH regions and two VL regions, yielding tandem scFvs (see e.g. Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8.).

Single domain antibodies comprise merely one (monomeric) antibody variable domain which is able to bind selectively to a specific antigen, independently of other V regions or domains. The first single domain antibodies were engineered from heavy chain antibodies found in camelids, and these are called V_{H}H fragments. Cartilaginous fishes also have heavy chain antibodies (IgNAR) from which single domain antibodies called V_{NAR} fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

A (single domain mAb)₂ is hence a monoclonal construct composed of (at least) two single domain monoclonal antibodies, which are individually selected from the group comprising VH, VL, VHH and V_{NAR}. The linker is preferably in the form of a peptide linker. Similarly, an “scFv-single domain mAb” is a monoclonal construct composed of at least one single domain antibody as described above and one scFv molecule as described above. Again, the linker is preferably in the form of a peptide linker.

It is also envisaged that the construct of the invention has, in addition to its function to bind to the target antigen CD33 and CD3, a further function. In this format, the construct is a trifunctional or multifunctional construct by targeting target cells through binding to the target antigen, mediating cytotoxic T cell activity through CD3 binding and providing a further function such as a label (fluorescent etc.), a therapeutic agent such as a toxin or radionuclide, etc.

Covalent modifications of the constructs are also included within the scope of this invention, and are generally, but not always, done post-translationally. For example, several types of covalent modifications of the construct are introduced into the molecule by reacting specific amino acid residues of the construct with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives.

Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the constructs of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the constructs included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of an construct is preferably altered through changes at the DNA level, particularly by mutating the DNA encoding the polypeptide at preselected bases such that codons are generated that will translate into the desired amino acids.

Another means of increasing the number of carbohydrate moieties on the construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the construct are contemplated herein. For example, another type of covalent modification of the construct comprises linking the construct to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the construct, e.g. in order to facilitate the addition of polymers such as PEG.

In some embodiments, the covalent modification of the constructs of the invention comprises the addition of one or more labels. The labelling group may be coupled to the construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, 35S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dye (including, but not limited to, chromophores, phosphors and fluorophores) such as fluorescent groups (e.g., FITC, rhodamine, lanthanide phosphors), chemilluminescent groups, and fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sites for secondary antibodies, metal binding domains, epitope tags, etc.) By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78. In one approach, recombinant fusion proteins comprising the target antigen antibody fragment or derivative fused to a leucine zipper peptide are expressed in suitable host cells, and the soluble oligomeric target antigen antibody fragments or derivatives that form are recovered from the culture supernatant.

The construct of the invention may also comprise additional domains, which are e.g. helpful in the isolation of the molecule or relate to an adapted pharmacokinetic profile of the molecule. Domains helpful for the isolation of an construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed constructs characterized by the identified CDRs are preferred to comprise a His-tag domain, which is generally known as a repeat of consecutive His residues in the amino acid sequence of a molecule, preferably of six His residues.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors

The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ζ ((zeta) chain to form the T cell receptor CD3 complex and to generate an activation signal in T lymphocytes. The CD3γ (gamma), CD3δ (delta), and CD3ε(epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. The sequence of a preferred human CD3 epsilon extracellular domain is shown in SEQ ID NO: 1, and the most preferred CD3 binding epitope corresponding to amino acid residues 1-27 of the human CD3 epsilon extracellular domain is represented in SEQ ID NO: 2.

The redirected lysis of target cells via the recruitment of T cells by a multispecific, at least bispecific, construct involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by bispecific constructs can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque target cell antigen, the effector cells should also be of macaque origin such as a macaque T cell line, e.g. 4119LnPx. The target cells should express (at least the extracellular domain of) target cell antigen, e.g. human or macaque target cell antigen. Target cells can be a cell line (such as CHO) which is stably or transiently transfected with target cell antigen, e.g. human or macaque target cell antigen. Alternatively, the target cells can be a target cell antigen positive natural expresser cell line, such as a human cancer cell line. Usually EC50 values are expected to be lower with target cell lines expressing higher levels of target cell antigen on the cell surface. The effector to target cell (E:T) ratio is usually about 10:1, but can also vary. Cytotoxic activity of bispecific constructs can be measured in a ⁵¹chromium release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays, ATP-based assays including bioluminescent assays, the sulforhodamine B (SRB) assay, WST assay, clonogenic assay and the ECIS technology.

The cytotoxic activity mediated by bispecific constructs of the present invention is preferably measured in a cell-based cytotoxicity assay. It is represented by the EC₅₀ value, which corresponds to the half maximal effective concentration (concentration of the construct which induces a cytotoxic response halfway between the baseline and maximum). Preferably, the EC₅₀ value of the bispecific constructs is ≤20.000 μg/ml, more preferably ≤5000 μg/ml, even more preferably ≤1000 μg/ml, even more preferably ≤500 μg/ml, even more preferably ≤350 μg/ml, even more preferably ≤250 μg/ml, even more preferably ≤100 μg/ml, even more preferably ≤50 μg/ml, even more preferably ≤10 μg/ml, and most preferably ≤5 μg/ml.

Any of the above given EC₅₀ values can be combined with any one of the indicated scenarios of a cell-based cytotoxicity assay, e.g. in line with the methods described in the appended example. For example, when (human) CD8 positive T cells or a macaque T cell line are used as effector cells, the EC₅₀ value of the bispecific construct of the invention (e.g. a target cell antigen/CD3 bispecific construct) is preferably 1000 μg/ml, more preferably 500 μg/ml, even more preferably 250 μg/ml, even more preferably 100 μg/ml, even more preferably 50 μg/ml, even more preferably 10 μg/ml, and most preferably ≤5 μg/ml. If in this assay the target cells are (human or macaque) cells transfected with the target antigen (e.g. the target cell antigen CD33), such as CHO cells, the EC₅₀ value of the bispecific construct is preferably 150 μg/ml, more preferably 100 μg/ml, even more preferably 50 μg/ml, even more preferably 30 μg/ml, even more preferably 10 μg/ml, and most preferably ≤5 μg/ml. If the target cells are a positive natural expresser cell line (e.g. of target cell antigen), then the EC₅₀ value is preferably 350 μg/ml, more preferably 250 μg/ml, even more preferably 200 μg/ml, even more preferably 100 μg/ml, even more preferably 150 μg/ml, even more preferably 100 μg/ml, and most preferably 50 μg/ml, or lower. When (human) PBMCs are used as effector cells, the EC₅₀ value of the bispecific construct is preferably 1000 μg/ml, more preferably 750 μg/ml, more preferably 500 μg/ml, even more preferably ≤350 μg/ml, even more preferably ≤250 μg/ml, even more preferably ≤100 μg/ml, and most preferably ≤50 μg/ml, or lower.

Preferably, the bispecific constructs of the present invention do not induce/mediate lysis or do not essentially induce/mediate lysis of target cell antigen negative cells such as CHO cells. The term “do not induce lysis”, “do not essentially induce lysis”, “do not mediate lysis” or “do not essentially mediate lysis” means that an constructs of the present invention does not induce or mediate lysis of more than 30%, preferably not more than 20%, more preferably not more than 10%, particularly preferably not more than 9%, 8%, 7%, 6% or 5% of target cell antigen negative cells, whereby lysis of a target cell antigen positive cell line is set to be 100%. This usually applies for concentrations of the construct of up to 500 nM. The skilled person knows how to measure cell lysis without further ado. Moreover, the present specification teaches specific instructions how to measure cell lysis.

Preferably, the bispecific construct for the use according to the invention is administered according to a schedule comprising the following steps:
(a) administration of a first dose of the bispecific construct, followed by
(b) administration of a second dose of the bispecific construct, wherein said second dose exceeds said first dose, followed by
(c) administration of a third dose of the bispecific construct, wherein said third dose exceeds said second dose, optionally—depending on the indication—followed by
(d) administration of a forth, a fifth and a sixth dose of the bispecific construct, wherein said optional forth dose exceeds said third dose, and said optional fifth dose exceeds the optional forth dose and the optional sixth dose exceeds the fifth dose.

In line with the above it is further preferred that the period of administration of the first dose is up to seven days. This period of administration of the first dose may be used during the initial phase/first cycle of administration of the bispecific construct e.g. to reduce the tumor load in a patient (tumor debulking) while avoiding conditions such as cytokine storm and/or cytokine release syndrome which one might expect in case a higher dose is used during the period of administration of the first dose.

While in one embodiment of the invention the period of administration of the first dose is up to seven days, it is also within this preferred embodiment that this first dose is administered for a period of six days, five days, four days, three days, two days or one day. In the case that the tumor load or general condition of the individual patient does require the administration of the limited dose of the bispecific construct in the first limited dose step, this first dose step is understood as a run-in phase/adaptation phase which should avoid or limit side effects resulting from the first contact of the patient with the bispecific construct. A preferred range for a dose in such run-in phase/adaptation phase may be in a range of 1 to 50 μg/d, preferably in a range of 3 to 30 μg/d, further preferably in a range of 4 to 20 μg/d and even more preferably in a range of 5 to 15 μg/d for a canonical BiTE® such as CD33×CD3 BISPECIFIC CONSTRUCT, which is a 54 kDa single chain polypeptide. In a very preferred embodiment, the bispecific construct according to the present invention is administered at a dose of 10 μg/d.

Preferred ranges for a second dose of the bispecific construct are e.g. for a canonical BiTE® such as CD33×CD3 BISPECIFIC CONSTRUCT in the range of 10 μg/d to 10 mg/d, more preferably in the range of 25 μg/d to 1 mg/d and even more preferably in the range of 30 μg/d to 500 μg/d. In a very preferred embodiment, the second dose is 30 μg/d or 60 μg/d. In line with the above, the preferred ranges for the third dose of the bispecific construct exceed the respective dose of the second dose. The third dose is typically in the range of 60 μg/d to 500 μg/d and preferably eradicates residual target cells which may have evaded treatment equivalent to the second dose according to the present invention.

It was surprisingly found that when a step dosing comprising at least two dosage steps is applied according to the present invention, then immunologic side effects such as undesired cytokine release, e.g. a cytokine release syndrome, may be effectively prevented. In contrast, if a dose equivalent to the second dosage is given without a prior lower dose equitant to the first dose of the present invention, then side effects, such as undesired cytokine release, e.g. a cytokine release syndrome, may occur. The same applies for the third dosage with respect to the second dosage.

It is also preferred for the present invention that the period of administration of the first and second dose is as short as possible to reach the target dose which addresses leukemic stem cells as soon as possible. This is decisive for therapeutic success with respect to an aggressive and progredient disease such as AML. Hence, it is a major achievement according to the present invention to provide a dosage scheme having a period of administration of the first dosage for only two or three days, preferably two days, and of two to four days for the second dosage. In turn the third dosage or the optional forth dosage, i.e. the target dosage, comprises a prolonged period of administration of preferably at least 21 days combined as described herein.

Also in line with the present invention it is preferred for the bispecific construct used in the treatment of myeloid leukemia that the first binding domain of the bispecific construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100 preferably 94 to 96 an 98 to 100 as described herein.

Moreover, in line with the present invention it is preferred for the bispecific construct used in the treatment of myeloid leukemia that the second binding domain of the bispecific construct comprises groups of six CDRs selected from the group consisting of SEQ ID NOs: 9 to 14, 27 to 32, 45 to 50, 63 to 68, 81 to 86, 99 to 104, 117 to 122, 135 to 140, 153 to 158 and 171 to 176 of WO 2008/119567.

As well as the second binding domain the first (or any further) binding domain(s) of the construct of the invention is/are preferably cross-species specific for members of the mammalian order of primates. Cross-species specific CD3 binding domains are, for example, described in WO 2008/119567. According to one embodiment, the first and second binding domain, in addition to binding to human CD33 target cell antigen and human CD3, respectively, will also bind to the CD33 target cell antigen/CD3 of primates including (but not limited to) new world primates (such as Callithrix jacchus, Saguinus Oedipus or Saimiri sciureus), old world primates (such baboons and macaques), gibbons, and non-human homininae. Callithrix jacchus and Saguinus oedipus are both new world primate belonging to the family of Callitrichidae, while Saimiri sciureus is a new world primate belonging to the family of Cebidae.

In a preferred embodiment of the invention the bispecific construct is a bispecific construct. In line with the definitions provided herein above, this embodiment relates to bispecific constructs, which are constructs. In a preferred embodiment of the invention the bispecific construct is a single chain construct. Such bispecific single chain construct may comprise in line with the invention an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108.

Amino acid sequence modifications of the bispecific constructs described herein are also contemplated. For example, it may be desirable to improve the binding affinity and/or other biological properties of the bispecific construct. Amino acid sequence variants of the bispecific constructs are prepared by introducing appropriate nucleotide changes into the bispecific constructs nucleic acid, or by peptide synthesis. All of the below described amino acid sequence modifications should result in a bispecific construct which still retains the desired biological activity (binding to target cell antigen and to CD3) of the unmodified parental molecule.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (GIy or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, GIu); a positively charged sidechain (e.g., Arg, His, Lys); or an uncharged polar side chain (e.g., Asn, Cys, GIn, GIy, His, Met, Phe, Ser, Thr, Trp, and Tyr).

Amino acid modifications include, for example, deletions from, and/or insertions into, and/or substitutions of, residues within the amino acid sequences of the bispecific constructs. Any combination of deletion, insertion, and substitution is made to arrive at the final construct, provided that the final construct possesses the desired characteristics. The amino acid changes also may alter post-translational processes of the bispecific constructs, such as changing the number or position of glycosylation sites.

For example, 1, 2, 3, 4, 5, or 6 amino acids may be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs. Preferably, amino acid sequence insertions include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. An insertional variant of the bispecific construct of the invention includes the fusion to the N-terminus or to the C-terminus of the bispecific construct to an enzyme or a fusion to a polypeptide which increases the serum half-life of the bispecific construct.

An increased half-life is generally useful in in vivo applications of immunoglobulins, especially antibodies and most especially antibody fragments of small size. Although such constructs based on antibody fragments (Fvs, disulphide bonded Fvs, Fabs, scFvs, dAbs) are able to rapidly reach most parts of the body, those constructs are likely to suffer from rapid clearance from the body. Strategies described in the art for extending the half-life of constructs such as single-chain diabodies include the conjugation of polyethylene glycol chains (PEGylation), the fusion to the IgG Fc region or to an albumin or albumin-binding domain.

Serum albumin is a protein physiologically produced by the liver; it occurs dissolved in blood plasma and is the most abundant blood protein in mammals. Albumin is essential for maintaining the oncotic pressure needed for proper distribution of body fluids between blood vessels and body tissues. It also acts as a plasma carrier by non-specifically binding several hydrophobic steroid hormones and as a transport protein for hemin and fatty acids. The term “serum albumin” respectively the human variant thereof (“human albumin”) defines in the context of the invented proteins either the parental human serum albumin protein (sequence as described in SEQ ID NO: 109) or any variant (e.g. such as albumin protein as depicted in SEQ ID NOs: 110-138) or fragment thereof preferably expressed as genetic fusion proteins and by chemical crosslinking etc. at least with one therapeutic protein. Variants comprising single or multiple mutations or fragments of albumin provide improved properties such as affinities to FcRn receptor and extended plasma half-life compared to its parent or reference. Variants of human albumin are described e.g. in WO 2014/072481. In line with the invention the serum albumin may be linked to the construct via a peptide linker. It is preferred that the peptide linker has the amino acid sequence (GGGGS)ₙ (SEQ ID NO: 13)ₙ wherein “n” is an integer in the range of 1 to 5. Further preferred is that “n” is an integer in the range of 1 to 3, and most preferably “n” is 1 or 2.

The sites of greatest interest for substitutional mutagenesis include the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the bispecific constructs that are preferred locations for mutagenesis is called “alanine scanning mutagenesis” as described by Cunningham and Wells in Science, 244: 1081-1085 (1989). Here, a residue or group of target residues within the bispecific construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions then are refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed bispecific construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of target antigen binding activities.

Generally, if amino acids are substituted in one or more or all of the CDRs of the heavy and/or light chain, it is preferred that the then-obtained “substituted” sequence is at least 60%, more preferably 65%, even more preferably 70%, particularly preferably 75%, more particularly preferably 80% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the bispecific construct may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 1, below) is envisaged as long as the bispecific construct retains its capability to bind to target cell antigen via the first binding domain and to CD3 epsilon via the second binding domain and/or its CDRs have an identity to the then substituted sequence (at least 60%, more preferably 65%, even more preferably 70%, particularly preferably 75%, more particularly preferably 80% identical to the “original” CDR sequence).

Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened for a desired characteristic.

TABLE 1
Amino acid substitutions

| Original | Exemplary Substitutions | Preferred Substitutions |
|---|---|---|
| Ala (A) | val, leu, ile | val |
| Arg (R) | lys, gln, asn | lys |
| Asn (N) | gln, his, asp, lys, arg | gln |
| Asp (D) | glu, asn | glu |
| Cys (C) | ser, ala | ser |
| Gln (Q) | asn, glu | asn |
| Glu (E) | asp, gln | asp |
| Gly (G) | Ala | ala |
| His (H) | asn, gln, lys, arg | arg |
| Ile (I) | leu, val, met, ala, phe | leu |
| Leu (L) | norleucine, ile, val, met, ala | ile |
| Lys (K) | arg, gln, asn | arg |
| Met (M) | leu, phe, ile | leu |
| Phe (F) | leu, val, ile, ala, tyr | tyr |
| Pro (P) | Ala | ala |
| Ser (S) | Thr | thr |
| Thr (T) | Ser | ser |
| Trp (W) | tyr, phe | tyr |
| Tyr (Y) | trp, phe, thr, ser | phe |
| Val (V) | ile, leu, met, phe, ala | leu |

Substantial modifications in the biological properties of the bispecific construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the bispecific construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the bispecific construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” is one with the specified homology, similarity, or identity to the parent CDR of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

In one embodiment the bispecific construct for the use in accordance with this invention is administered in combination with one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the one or more epigenetic factors are administered prior to the administration of the bispecific construct;
(b) the one or more epigenetic factors are administered subsequent to the administration of the bispecific construct; or
(c) the one or more epigenetic factors and the bispecific construct are administered simultaneously.

The term “epigenetic factor” in connection with the present invention defines a compound which is capable of changing the gene expression or cellular phenotype of a cell population upon administration. It is understood that such change refers to one or more functional relevant modifications to the genome without involving a change in the nucleic acid sequence. Examples of such modifications are DNA methylation and histone modification, which are both important for the regulation of gene expression without altering the underlying DNA sequence.

Details for a treatment of myeloid leukemia comprising the administration of the bispecific construct in combination with one or more of the above described epigenetic factors have been provided in PCT/EP2014/069575.

In one embodiment of the invention it is preferred that the one or more epigenetic factors are administered up to seven days prior to the administration of the bispecific construct.

Also in one embodiment of the invention it is preferred that the epigenetic factor is hydroxyurea.

It is preferred for the present invention that the myeloid leukemia is selected from the group consisting of acute myeloblastic leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and acute biphenotypic leukaemia. More preferably, the myeloid leukemia is an acute myeloid leukemia (AML). The definition of AML inter alia comprises acute myeloblastic leukemia, acute myeloid dendritic cell leukemia, acute myelomonocytic leukemia, acute basophilic leukemia, acute eosinophilic leukemia, acute megakaryoblastic leukemia, acute erythroid leukemia, and acute panmyeloic leukemia

The bispecific construct described in connection with this invention may be formulated for an appropriate administration to a subject in the need thereof in form of a pharmaceutical composition. Formulations described herein are useful as pharmaceutical compositions in the treatment, amelioration and/or prevention of the pathological medical condition as described herein in a patient in need thereof. The term “treatment” refers to both therapeutic treatment and prophylactic or preventative measures. Treatment includes the application or administration of the formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease.

The term “disease” refers to any condition that would benefit from treatment with the bispecific construct or the pharmaceutical composition described herein. This includes chronic and acute disorders or diseases including those pathological conditions that predispose the mammal to the disease in question.

The terms “subject in need” or those “in need of treatment” includes those already with the disorder, as well as those in which the disorder is to be prevented. The subject in need or “patient” includes human and other mammalian subjects that receive either prophylactic or therapeutic treatment.

The bispecific construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include, but are not limited to
topical routes (such as epicutaneous, inhalational, nasal, ophthalmic, auricular/aural, vaginal, mucosal);
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).

The pharmaceutical compositions and the bispecific construct described in connection with the invention are particularly useful for parenteral administration, e.g., subcutaneous or intravenous delivery, for example by injection such as bolus injection, or by infusion such as continuous infusion. Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

In particular, the present invention provides for an uninterrupted administration of the suitable composition. As a non-limiting example, uninterrupted or substantially uninterrupted, i.e. continuous administration may be realized by a small pump system worn by the patient for metering the influx of therapeutic agent into the body of the patient. The pharmaceutical composition comprising the bispecific construct described in connection with the invention can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the invention together make up one “uninterrupted administration” of such therapeutic agent.

The continuous or uninterrupted administration of the bispecific construct described in connection with the invention may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may also be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

The compositions of the present invention can be administered to the subject at a suitable dose which can be determined e.g. by dose escalating studies by administration of increasing doses of the bispecific construct described in connection with the invention exhibiting cross-species specificity described herein to non-chimpanzee primates, for instance macaques. As set forth above, the bispecific construct described in connection with the invention exhibiting cross-species specificity described herein can be advantageously used in identical form in preclinical testing in non-chimpanzee primates and as drug in humans.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts or doses effective for this use will depend on the condition to be treated (the indication), the delivered bispecific construct, the therapeutic context and objectives, the severity of the disease, prior therapy, the patient's clinical history and response to the therapeutic agent, the route of administration, the size (body weight, body surface or organ size) and/or condition (the age and general health) of the patient, and the general state of the patient's own immune system.

A therapeutic effective amount of a bispecific construct described in connection with the invention preferably results in a decrease in severity of disease symptoms, an increase in frequency or duration of disease symptom-free periods or a prevention of impairment or disability due to the disease affliction. For treating target cell antigen-expressing tumors, a therapeutically effective amount of the bispecific construct described in connection with the invention, e.g. an anti-target cell antigen/anti-CD3 construct, preferably inhibits cell growth or tumor growth by at least about 20%, at least about 40%, at least about 50%, at least about 60%, at least about 70%, at least about 80%, or at least about 90% relative to untreated patients. The ability of a compound to inhibit tumor growth may be evaluated in an animal model predictive of efficacy in human tumors.

The pharmaceutical composition can be administered as a sole therapeutic or in combination with additional therapies such as anti-cancer therapies as needed, e.g. other proteinaceous and non-proteinaceous drugs. These drugs may be administered simultaneously with the composition comprising the bispecific construct described in connection with the invention as defined herein or separately before or after administration of said bispecific construct in timely defined intervals and doses. Further, the present inventors observed that rare side effects, such as immunologic side effects could be prevented or alleviated by means of a glucocorticoid (pre) and/or (co)therapy.

Accordingly, the present invention establishes that glucocorticoids such as dexamethasone mitigate or even prevent adverse effects which might occur in the course of a treatment with CD33/CD3 specific bispecific constructs according to the present invention.

Glucocorticoids (GCs) are still the most widely used immunosuppressive agents for the treatment of inflammatory disorders and autoimmune diseases. Glucocorticoids (GC) are a class of steroid hormones that bind to the glucocorticoid receptor (GR), which is present in almost every vertebrate animal cell, including humans. These compounds are potent anti-inflammatory agents, regardless of the inflammation's cause. Glucocorticoids suppress, inter alia, the cell-mediated immunity by inhibiting genes that code for the cytokines IL-1, IL-2, IL-3, IL-4, IL-5, IL-6, IL-8 and IFN-γ.

Cortisone which belongs to the group of GCs is an important therapeutic drug which is used to fight many ailments ranging from Addison's disease to rheumatoid arthritis. Ever since the discovery of its anti-rheumatic properties, which led to its acclaim as a wonder drug, many derivatives of cortisone with enhanced properties to better fight a specific ailment have been produced. Cortisone belongs to a group of steroids known as corticosteroids. These steroids are produced by the adrenal cortex, which is the outer part of the adrenal glands, near the kidneys. The corticosteroids are divided into two main groups: the glucocorticoids (GCs), which control fat, protein, calcium and carbohydrate metabolism, and the mineralocorticoids controlling sodium and potassium levels. Cortisone belongs to the former group, i.e. to the GCs. Cortisone and its many derivatives are used for a variety of diseases. Cortisone also helped to make organ transplants a reality due to its ability to minimize the defense reaction of the body towards foreign proteins present in the implanted organ and thus damage the functionality of the implanted organ. However, despite clinical use during more than 50 years, the specific anti-inflammatory effects of GC on different cellular compartments of the immune system are not yet clear. GCs affect nearly every cell of the immune system, and there is growing evidence for cell type-specific mechanisms.

In one specific embodiment, the present invention relates to a glucocorticoid (GC) for use in the amelioration, treatment or prophylaxis of adverse effects caused by a CD33/CD3 bispecific construct. As outlined above, these unwanted adverse effects may be prevented by a step dosing as described herein. However, for mere precaution, glucocorticoid(s) for use in the amelioration, treatment or prophylaxis of (immunological) adverse effects in a patient may be provided wherein said patient is subject to therapy with a CD33/CD3 bispecific construct. Accordingly, in one further aspect the present invention relates to a glucocorticoid (GC) for use in a method in the amelioration, treatment or prophylaxis of immunological adverse effects caused by a CD33/CD3 bispecific construct according to the present invention.

Also, the present invention relates to a method of amelioration, treatment or prophylaxis of immunological adverse effects caused by a CD33/CD3 bispecific construct, said method comprising administering to a patient in need thereof IL-6R blocking antibody tocilizumab or a glucocorticoid (GC). The GC is preferably administered in an amount which is sufficient to ameliorate, treat or prevent said immunological adverse effects caused by a CD33/CD3 bispecific construct.

The term “glucocorticoid” means compounds that bind, preferably specifically, to the glucocorticoid receptor. Said term includes compound(s) selected from the group consisting of cortisone, cortisol (hydrocortisone), cloprednol, prednisone, prednisolone, methylprednisolone, deflazacort, fluocortolone, triamcinolone, dexamethasone, betamethasone, cortivazol, paramethasone, and/or fluticasone, including pharmaceutically acceptable derivatives thereof. In the context of the embodiments of the present invention, the mentioned compounds may be used alone or in combination. Dexamethasone is preferred. The present invention is however not limited to the above mentioned specific GCs. It is envisaged that all substances which already are or will be classified as a “glucocorticoid”, may be employed in the context of the present invention as well. Such future glucocorticoids include compounds which specifically bind to and activate the glucocorticoid receptor. The term “specifically binds to the GC receptor” means in accordance with the present invention that the GC (or a compound which is assumed to act like a GC) associates with (e.g., interacts with) the GC receptor (also known as NR3C1) to a statistically significant degree as compared to association with proteins/receptors generally (i.e., non-specific binding). When the GC receptor binds to glucocorticoids, its primary mechanism of action is the regulation of gene transcription. In the absence of GC, the glucocorticoid receptor (GR) resides in the cytosol complexed with a variety of proteins including heat shock protein 90 (hsp90), the heat shock protein 70 (hsp70) and the protein FKBP52 (FK506-binding protein 52). The binding of the GC to the glucocorticoid receptor (GR) results in release of the heat shock proteins. It is thus envisaged that a future GC, or a pharmaceutically acceptable derivative or salt of a GC is preferably able to bind to the GC receptor and to release the above mentioned heat shock proteins. The activated GR complex up-regulates the expression of anti-inflammatory proteins in the nucleus or represses the expression of pro-inflammatory proteins in the cytosol (by preventing the translocation of other transcription factors from the cytosol into the nucleus).

In a preferred embodiment, said GC is selected from the most clinical used and relevant GCs like dexamethasone, fluticasonepropionate, prednisolone, methylprednisolone, betamethasone, triamcinolonacetonide or combinations thereof.

In an even more preferred embodiment, said GC is dexamethasone.

Dexamethasone has the highest glucocorticoid potency of the most commonly used steroids and also has the longest half-life (see Table 2 below). But a person skilled in the field can select one of the other known glucocorticoids, some of which are disclosed herein, and select an appropriate effective dose to ameliorate or prevent immunological adverse events that may result from the treatment of a patient in need thereof.

TABLE 2
steroid dosing

| | | Relative | Relative | |
|---|---|---|---|---|
| | Approx. | anti-inflammatory | mineralocorticoid | Biologic |
| | equiv. dose | (glucocorticoid) | (Na⁺ retaining) | half-life |
| Agent | (mg) | potency | potency | (hrs) |
| Cortisone | 25 | 0.8 | 0.8 | 8-12 |
| Hydrocortisone | 20 | 1 | 1 | 8-12 |
| Prednisone | 5 | 4 | 0.8 | 18-36 |
| Prednisolone | 5 | 4 | 0.8 | 18-36 |
| Methylprednisolone | 5 | 5 | 0.5 | 18-36 |
| Dexamethasone | 0.75 | 25 | 0 | 36-54 |

Dexamethasone also possesses a beneficial effect in malignant central nervous system (CNS) disease (e.g. CNS lymphoma or brain metastases)—possibly due to specific penetration to the CNS. It is also preferentially (over other steroids) used to treat brain edema. Although corticosteroids decrease capillary permeability in the tumor itself, it has been found in animal models that dexamethasone may act differently and decrease edema by effects on bulk flow away from the tumor (Molnar, Lapin, & Goothuis, 1995, Neurooncol. 1995; 25(1):19-28.

For the clinical trials in connection with the application of a CD33/CD3 bispecific construct, the present inventors had to develop a treatment regime which was efficient and would be well tolerated by most of the patients. To this end, the present inventors applied a step-wise application of a CD33/CD3 bispecific construct as outlined herein. Thereby, adverse effects could be reduced in number, ameliorated and even prevented.

The dose of the GC that is to be used in accordance with the embodiments of the present invention is not limited, i.e. it will depend on the circumstances of the individual patient. GC can be administered intravenously or orally. Preferred dosages of the GC include, however, between 1 to 6 mg (dexamethasone equivalent) at the lower end of dosing to 40 mg (dexamethasone equivalent). Said dosage can be administered all at once or subdivided into smaller dosages. Preferred is a subdivide dose wherein one dose of GC is given prior to the infusion of the first and/or second dose according to the step dosing as described herein, and the other dose of GC is given prior to the administration of the second or third dose according to the step dosing as described herein. Hence, GC is preferably two times dosed per treatment cycle. Even more preferably, GC is administered one 24 or 8 h or 4 h or 1 h before the beginning of a treatment cycle or the beginning of the administration of the next higher dose within said treatment cycle. In this regard, 1 h is most preferred. The dose is 1 to 40 mg each, preferably 5 to 20 mg, most preferably 8 mg each. “d” denotes one day. Further dosage regimens are derivable from the appended examples. All dosages given in this paragraph refer to dexamethasone equivalents.

The term “effective and non-toxic dose” as used herein refers to a tolerable dose of a bispecific construct which is high enough to cause depletion of pathologic cells, tumor elimination, tumor shrinkage or stabilization of disease without or essentially without major toxic effects. Such effective and non-toxic doses may be determined e.g. by dose escalation studies described in the art and should be below the dose inducing severe adverse side events (dose limiting toxicity, DLT).

Alternatively, tocilizumab may be used in premedication.

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviations to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v4 (CTCAE). Laboratory parameters which may be tested include for instance hematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

In a preferred embodiment of the method of the invention only the first cycle of the treatment comprises the administration according to step (a), whereas the following cycles start with the dose according to step (b), (c) or (d).

It is preferred for the method of the invention that the first binding domain of the bispecific construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100.

Also in line with a preferred embodiment of the method of the invention the second binding domain of the bispecific construct comprises groups of six CDRs selected from the group consisting of SEQ ID NOs: 9 to 14, 27 to 32, 45 to 50, 63 to 68, 81 to 86, 99 to 104, 117 to 122, 135 to 140, 153 to 158 and 171 to 176 of WO 2008/119567.

In a preferred embodiment of the method of the invention the bispecific construct is a bispecific construct.

Moreover, it is preferred for the method of the invention that the bispecific construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108.

In one embodiment of the method of the invention the bispecific construct is administered in combination with one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the one or more epigenetic factors are administered prior to the administration of the bispecific construct;
(b) the one or more epigenetic factors are administered subsequent to the administration of the bispecific construct; or
(c) the one or more epigenetic factors and the bispecific construct are administered simultaneously.

It is preferred for the method of the invention that the one or more epigenetic factors are administered up to seven days prior to the administration of the bispecific construct.

For one embodiment of the method of the invention it is preferred that the epigenetic factor is hydroxyurea

As described herein above, in line with the present invention the myeloid leukemia is selected from the group consisting of acute myeloblastic leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and acute biphenotypic leukaemia. It is preferred that the myeloid leukemia is an acute myeloid leukemia (AML).

Also in one embodiment the invention provides a use of a bispecific antibody construct comprising a first binding domain specifically binding to CD33 and a second binding domain specifically binding to CD3 preferably for the preparation of a pharmaceutical composition for the treatment of myeloid leukemia, wherein the bispecific construct is to be administered for more than 14 days followed by a period of at least 14 days without administration of the construct.

It is preferred of the use of the invention that the bispecific construct is to be administered according to a schedule comprising the following steps:
(a) administration of a first dose of the bispecific construct, followed by
(b) administration of a second dose of the bispecific construct, wherein said second dose exceeds said first dose, followed by
(c) administration of a third dose of the bispecific construct, wherein said optional third dose exceeds said second dose, optionally followed by
(d) administration of a forth dose of the bispecific construct, wherein said optional third dose exceeds said third dose.

In a preferred embodiment of the use of the invention only the first cycle of the treatment comprises the administration according to step (a), whereas the following cycles start with the dose according to step (b), (c) or (d).

It is preferred for the use of the invention that the first binding domain of the bispecific construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88,94 to 96 an 98 to 100.

Also in line with a preferred embodiment of the use of the invention the second binding domain of the bispecific construct comprises groups of six CDRs selected from the group consisting of SEQ ID NOs: 9 to 14, 27 to 32, 45 to 50, 63 to 68, 81 to 86, 99 to 104, 117 to 122, 135 to 140, 153 to 158 and 171 to 176 of WO 2008/119567.

In a preferred embodiment of the use of the invention the bispecific construct is a bispecific construct. Moreover, it is preferred for the use of the invention that the bispecific construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108.

In one embodiment of the use of the invention the bispecific construct is administered in combination with one or more epigenetic factors selected from the group consisting of histone deacetylase (HDAC) inhibitors, DNA methyltransferase (DNMT) I inhibitors, hydroxyurea, Granulocyte-Colony Stimulating Factor (G-CSF), histone demethylase inhibitors and ATRA (All Trans-retinoic acid) and wherein:
(a) the one or more epigenetic factors are administered prior to the administration of the bispecific construct;
(b) the one or more epigenetic factors are administered subsequent to the administration of the bispecific construct; or
(c) the one or more epigenetic factors and the bispecific construct are administered simultaneously.

It is preferred for the use of the invention that the one or more epigenetic factors are administered up to seven days prior to the administration of the bispecific construct.

For one embodiment of the use of the invention it is preferred that the epigenetic factor is hydroxyurea As described herein above, in line with the present invention the myeloid leukemia is selected from the group consisting of acute myeloblastic leukemia, chronic neutrophilic leukemia, myeloid dendritic cell leukemia, accelerated phase chronic myelogenous leukemia, acute myelomonocytic leukemia, juvenile myelomonocytic leukemia, chronic myelomonocytic leukemia, acute basophilic leukemia, acute eosinophilic leukemia, chronic eosinophilic leukemia, acute megakaryoblastic leukemia, essential thrombocytosis, acute erythroid leukemia, polycythemia vera, myelodysplastic syndrome, acute panmyeloic leukemia, myeloid sarcoma, and acute biphenotypic leukaemia. It is preferred that the myeloid leukemia is an acute myeloid leukemia (AML).

The patient population considered susceptible for the present inventive method is AML as defined by the WHO Classification persisting or recurring following one or more treatment courses except promyelocytic leukemia (APML). The patient population may comprise AML secondary to prior myelodysplastic syndrome. Preferably, the patient population comprises AML as defined by the WHO Classification either persisting/refractory after at least 1 primary induction courses (i.e., no response after at least 1 prior chemotherapy cycles) or recurring after having achieved an initial response to chemotherapy except promyelocytic leukemia (APML) and except AML secondary to prior myelodysplastic syndrome. Further, the preferred patient population is characterized by having more than 1% blasts in bone marrow, preferably more than 5% blasts. Typically, patient population ECOG performance status is less than 2.

### General Definitions

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that the inventions herein are not limited to particular methodology, protocols, or reagents, as such can vary. The discussion and examples provided herein are presented for the purpose of describing particular embodiments only and are not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

The following examples are provided for the purpose of illustrating specific embodiments or features of the present invention. These examples should not be construed as to limit the scope of this invention. The examples are included for purposes of illustration, and the present invention is limited only by the claims.

### Example 1

The objective of this study was to establish safety and tolerability of an exemplary CD33×CD3 bispecific construct (SEQ ID NO: 104) and identify phase 2 recommended dose

### Study Design and Patients

The present study is a first-in-human, open label, nonrandomized, multicentre, phase 1, sequential dose-escalation study (NCT02520427). Each cycle (2-4 weeks) was followed by an infusion-free interval. Key inclusion criteria were male or female (≥18 years old) patients with confirmed relapsed/refractory (R/R) AML diagnosis, >5% myeloblasts in bone marrow (BM), Eastern Cooperative Oncology Group performance status score ≤2, and patients with ≥1 prior therapies including hematopoietic stem cell transplantation (HSCT) Assessments and Dose-steps

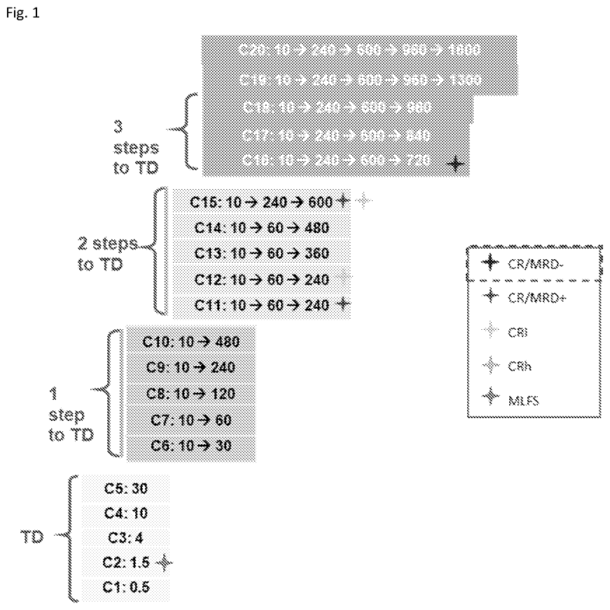

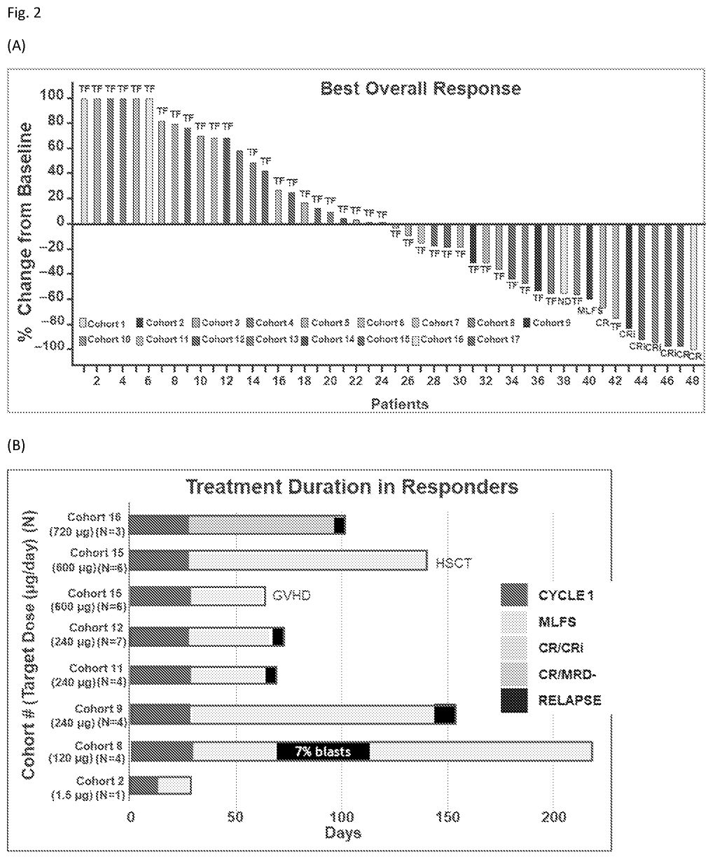

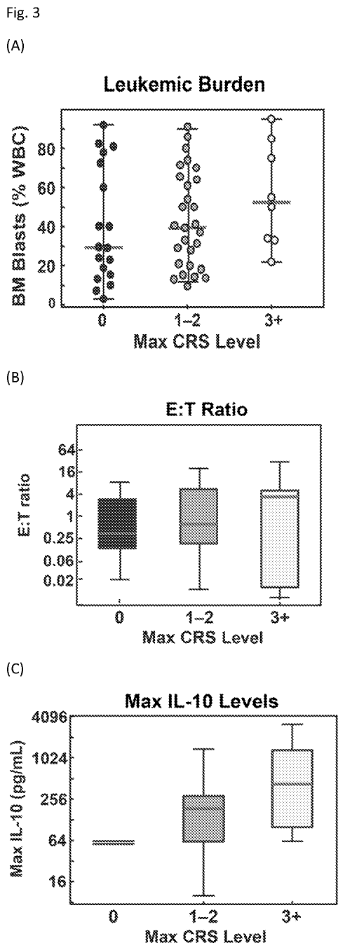

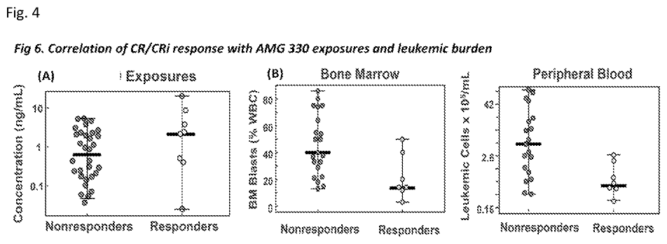

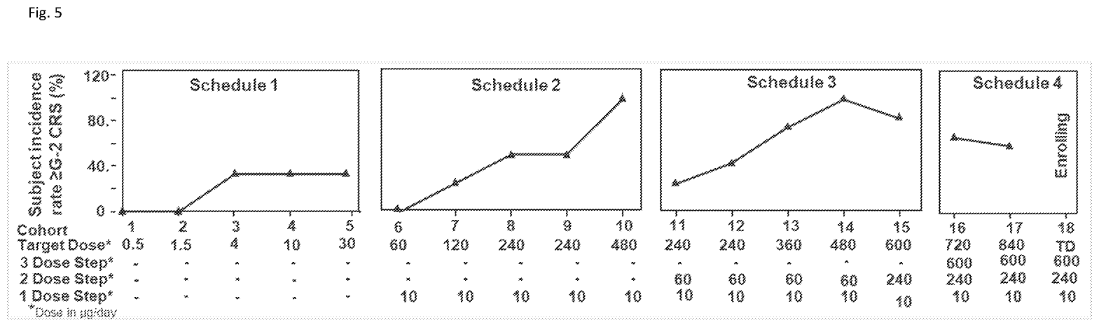

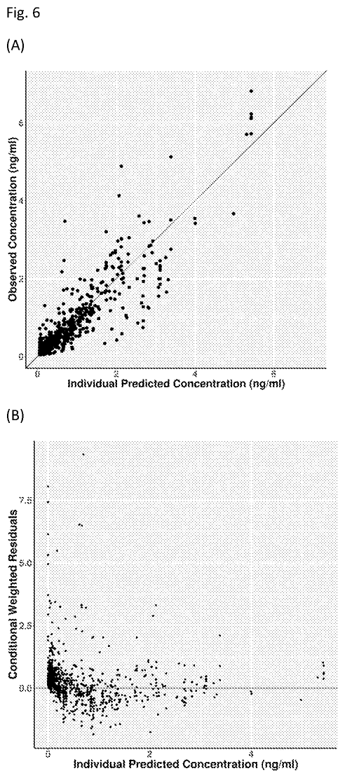

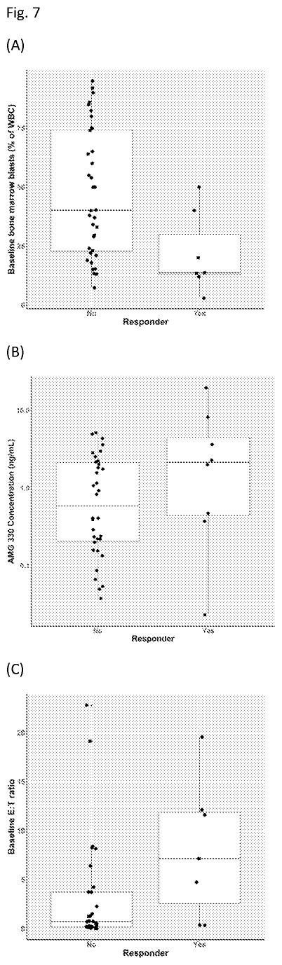

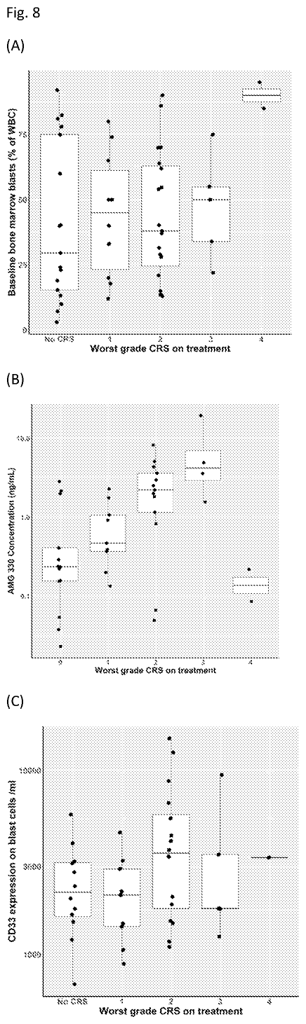

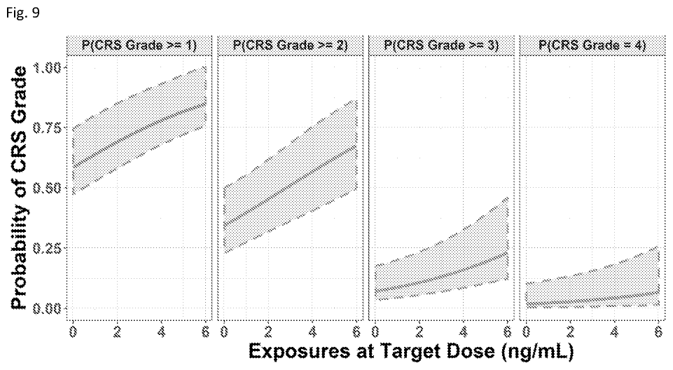

The molecule was evaluated as a cIV infusion using a 3+3 design. Response was assessed per revised International Working Group criteria. Dose steps were tested at 10 μg (1st step; cohorts 6-10), 60 g and 240 μg (2^{nd} step; cohorts 11-15), and 600 μg (3rd step; cohorts 16-18) (FIG. 1). Dose steps were intermediate doses of the molecule administered with 1-5 day/s interval prior to the target dose.

Statistics: Descriptive statistics was used for demographics, safety, pharmacokinetic, and pharmacodynamic data

### Results

TABLE 3
Demographics and baseline disease characteristics summarized

| Characteristics | Patients N = 60 | |
|---|---|---|
| Median age (years) | 58 | (18-80) |
| Sex, n (%) | | |
| Male | 33 | (55) |
| ELN risk classification, n (%) | | |
| Favourable | 4 | (7) |
| Intermediate | 9 | (15) |
| Adverse | 37 | (62) |
| Prior lines of therapies, n (%) | | |
| 1 | 9 | (15) |
| 2 | 11 | (18) |
| 3 | 11 | (18) |
| ≥4 | 29 | (48) |
| AML type, n (%) | | |
| AML, NOS | 37 | (61) |
| AML with recurrent genetic abnormalities | 16 | (27) |
| AML with myelodysplasia-related changes | 7 | (12) |
| ECOG PS, n (%) | | |
| 0 | 15 | (25) |
| 1 | 39 | (65) |
| 2 | 5 | (8) |
| Baseline leukemia burden, n (%) | | |
| <25% | 19 | (32) |
| 25%-50% | 17 | (28) |
| >50% | 24 | (40) |
| Baseline myelosuppression (≥Gr 3), n (%) | | |
| Thrombocytopenia | 45 | (75) |
| Leukopenia | 35 | (58) |
| Neutropenia | 52 | (87) |
| Response to prior therapies, n (%) | 44 | (73) |
| Prior HSCT, n (%) | 27 | (45) |

AML, acute myeloid leukemia;
ECOG PS, Eastern Cooperative Oncology Group performance status;
ELN, European LeukemiaNet;
Gr, grade;
HSCT, haematopoietic stem cell transplantation;
N, total number of patients in the analysis set;
n, total number of patients with observed data;
NOS, not otherwise specified

TABLE 4
Key Drug-Related Adverse Events in ≥20% Patients

| | All grades, | | ≥Gr 3, | |
|---|---|---|---|---|
| Drug-Related* AEs, N = 60 | n (%) | | n (%) | |
| Patients with drug-related AEs | 54 | (90) | 33 | (55) |
| Immune disorders | | | | |
| Cytokine Release Syndrome | 40 | (67) | 9 | (15) |
| Skin disorders | 35 | (58) | 6 | (10) |
| (including rashes, pruritus, urticaria) | | | | |
| Gastrointestinal disorders | 18 | (30) | | |
| Nausea | 13 | (22) | 0 | (0) |
| Vomiting | 7 | (12) | 0 | (0) |
| Diarrhea | 4 | (7) | 0 | (0) |
| Elevated liver function tests | 15 | (25) | 3 | (5) |
| (including ALT, AST, ALP, GGT, bilirubin) | | | | |
| Blood and lymphatic disorders | 14 | (23) | | |
| Febrile neutropenia | 11 | (18)** | 10 | (17) |

*As per investigators' assessment;
**Database cleaning in progress for 1 AE in grade 2;
AE, adverse events;
ALP, alkaline phosphatase;
ALT, alanine aminotransferase;
AST, aspartate aminotransferase;
GGT, gamma glutamyltransferase;
Gr, grade;
N, total number of patients in the analysis set;
n, total number of patients with observed data

Cytokine Release Syndrome (CRS) was the most frequent (67%) CD33×CD3 bispecific construct (as exemplified by SEQ ID NO 104)-related adverse event (AE). Other frequent AEs reported in ≥40% patients included rashes (58%).

Higher grades of CRS were observed in patients with higher leukemic burden and with higher Effector:Target (E:T) ratio (FIGS. 3A and 3B). Frequency and severity of CRS was associated with higher levels of cytokines released in response to CD33×CD3 bispecific construct (as exemplified by SEQ ID NO 104) treatment (FIG. 3C).

Eight patients responded to SEQ ID NO 104 treatment: complete remission (CR; n=3, cohorts 11, 15, and 16), complete remission with incomplete hematologic recovery (CRi; n=4, cohorts 8, 9, 12, and 15) and morphologic leukemia-free state (n=1, cohort 2) with 3 responders out of 14 treated patients (21%) in cohorts 15-17 (FIG. 2). For patients achieving CR/CRi (n=7), minimal efficacious dose was established at the 120 μg/day dose level.

TABLE 5
Characteristics of the responders

| | Non-responders^{a} | Responders^{b} at |
|---|---|---|
| | at TD ≥120 μg, | TD ≥120 μg, |
| Characteristics | N = 35; n (%) | N = 7; n (%) |
| ELN risk classification | | |
| Favourable | 2 (6) | 0 |
| Intermediate | 3 (9) | 2 (29) |
| Adverse | 22 (63) | 4 (57) |
| Unknown | 8 (23) | 1 (14) |
| Lines of prior therapies | | |
| 1 | 5 (14) | 1 (14) |
| 2 | 5 (14) | 2 (29) |
| 3 | 6 (17) | 1 (14) |
| ≥4 | 19 (54) | 3 (43) |
| Prior HSCT | 14 (40) | 4 (57) |

^{a}Non-Responders (CR or CRi) at target dose ≥120 μg were patients with a best overall response other than CR or CRi on study and in cohort 8 or later cohorts with planned target dose ≥120 μg;
^{b}Responders (CR/CRi) at target dose ≥120 μg were patients with a best overall response of CR/CRi on study and in cohort 8 or later cohorts with planned target dose ≥120 μg.
Css, concentration at steady state;
BM, bone marrow;
ELN, European LeukemiaNet;
HSCT, hematopoietic stem cell transplantation;
N, total number of patients in the analysis set;
n, total number of patients with observed data;
TD, target dose;
WBC, white blood cells

Responders typically showed higher SEQ ID NO 104 Css than non-responders (FIG. 4A). Among patients with CR/CRi response (17%), 57% had adverse cytogenetic profile. Lower leukemic burden in bone marrow and peripheral blood was associated with higher likelihood of response to CD33×CD3 bispecific construct (as exemplified by SEQ ID NO 104) (FIG. 4B & 6C). 20% (4/20) of patients with ≤4 lines of prior therapies and 14% (3/22) of patients with ≥4 lines of prior therapies responded to CD33×CD3 bispecific construct (as exemplified by SEQ ID NO 104) treatment (Table 3). Among responders, 43% received ≥4 lines of prior therapies (Table 3)

ConclusionCD33×CD3 bispecific construct (as exemplified by SEQ ID NO 104) was safe and tolerable in R/R AML patients in the present study with CRS as the most frequent expected mechanism of action-based toxicity, and without unexpected toxicities to date

Optimized schedule of CD33×CD3 bispecific construct (as exemplified by SEQ ID NO 104) administration using dose steps allowed higher Target Dose levels with improved drug exposure. Frequency and severity of CRS was associated with higher levels of cytokines released in response to CD33×CD3 bispecific construct (as exemplified by SEQ ID NO 104) treatment, and higher CRS grades were observed in patients with higher baseline leukemic burden. A total of seven patients achieved CR/CRi at a minimal efficacious dose of at least 120 μg/day with a 21% response rate for patients in three cohorts 15-17.

### Example 2

The objectives of this study was to characterize the clinical pharmacokinetics, exposure-efficacy and exposure-safety relationships of CD33×CD3 bispecific construct (as exemplified by SEQ ID NO 104) in R/R AML patients using the data from phase I dose-escalation study (NCT02520427) and to evaluate the effect of baseline patient characteristics on efficacy and safety of SEQ ID NO 104.

### Methods

A continuous IV infusion of CD33×CD3 bispecific construct (as exemplified by SEQ ID NO 104) was evaluated in a 14-28 days cycle at escalating target doses (range from 0.5 to 720 μg/day) using a 3+3 design with patients receiving step dose/s (FIG. 2) Pharmacokinetics

Serum concentrations of CD33×CD3 bispecific construct (as exemplified by SEQ ID NO 104) were tested using a validated, GLP compliant, electro-chemiluminescence assay. Non-compartmental and population-based approach using nonlinear mixed effects modeling was used to characterize PK

A one compartment model with interindividual variability (IIV) on clearance and volume of distribution was used to describe the data

### Exposure-Response/Safety Analyses

Relationships between CD33×CD3 bispecific construct (as exemplified by SEQ ID NO 104) exposures, baseline patient characteristics with efficacy (IWG responses)/incidence of cytokine release syndrome (CRS) events were explored. Worst grade CRS for each patient was modelled with CD33×CD3 bispecific construct (as exemplified by SEQ ID NO 104) exposures using a proportional odds logistic regression model. The effect of baseline patient characteristics was tested as a covariate in the logistic regression model.

Data from 55 patients (sex: 33M/22F; median age of 58 [18-80] years) were included in the analyses. 8 responses were reported: CR (n=3, cohorts 11, 15, and 16), CRi (n=4, cohorts 8, 9, 12, and 15) and MLFS (n=1, cohort 2). CRS, an expected on-target toxicity, was the most frequent (67% all grades; 15% Gr ≥3) CD33×CD3 bispecific construct (as exemplified by SEQ ID NO 104)-related adverse event.

A one compartment model with linear clearance and IIV on CL and V was used to describe the CD33×CD3 bispecific construct PK (as exemplified by SEQ ID NO 104).

TABLE 6
PK parameters and variability estimates

| | Estimate | IIV CV |
|---|---|---|
| Parameter | (% RSE) | % (% RSE) |
| Clearance (L/day) | 180 (24.1) | 55.5 (87) |
| Volume of distribution (L) | 28.7 (70) | 111.8 (101) |
| σ1 (Proportional) | 0.38 (25) | |
| σ2 (Additive) | 0.19 (22.7) | |

In FIG. 6, Goodness of fit plots are shown (Observed vs Individual predicted concentrations, and Conditional weighted residuals vs Individual predicted concentrations) which were generated to check the model fit Results Dose dependent increases were observed in CD33×CD3 bispecific construct (as exemplified by SEQ ID NO 104) exposures.

A trend towards better clinical responses was observed in patients with lower baseline leukemic burden in bone marrow, higher CD33×CD3 bispecific construct (as exemplified by SEQ ID NO 104) exposures and higher baseline Effector (CD3): Target (Blasts) cell ratio (see FIG. 7)

Higher grade CRS events were observed in patients at higher baseline leukemic burden and CD33 expression on blast cells (see FIG. 8) A positive relationship was observed for CD33×CD3 bispecific construct (as exemplified by SEQ ID NO 104) exposures with probability of CRS occurrence and severity (see FIG. 9).

### Conclusions

Dose-related increases in CD33×CD3 bispecific construct (as exemplified by SEQ ID NO 104) exposures, lack of any major impact of shed target on free CD33×CD3 bispecific construct (as exemplified by SEQ ID NO 104) exposures & modest trends of exposure-efficacy and exposure-safety relationships were observed

Results from these analyses are being used to identify optimal CD33×CD3 bispecific construct (as exemplified by SEQ ID NO 104) dosing regimens that minimize the risk for CRS in ongoing and planned clinical investigations.

### Example 3

The objectives of this study was to characterize the clinical efficacy of CD33×CD3 bispecific construct (as exemplified by SEQ ID NO 104) in MRD+ AML patients using the data from phase I dose-escalation study (NCT02520427). Patients were screened for blast percentage at baseline and after the respective treatment cycle and peripheral blood cell counts at baseline and after the respective treatment cycle. Based on blast count, MRD status (“+” or “−”) was determined according to European LeukemiaNet (ELN) recommendations with 0.1% blast threshold (Schuurhuis G J, Heuser M, Freeman S, et al. Minimal/measurable residual disease in AML: a consensus document from the European LeukemiaNet MRD Working Party. Blood. 2018; 131:1275-1291). Subjects were pre-treated with 8 mg IV dexamethasone within 1 hour prior to the initial dose step of the CD33×CD3 bispecific construct (as exemplified by SEQ ID NO 104). Then, subjects were treated with a starting dose of 30 μg/d for 2 days, then with a dose of 240 μg/d for 5 days and then with a target dose of 600, 720, 840, 960, and 1600 μg/d for 21 days.

Three subjects completed cycle 1 with a total duration of 28 days being treated at dose of 30 μg/d for 2 days, 240 μg/d for 5 days and 600 μg/d for 21 days. One subject #66003-044 enrolled with MRD+ CRi with 4.2% blasts at baseline; after completion of cycle 1, the number of blasts by morphology was 0%, MRD assessment showed abnormal blasts <0.01% meeting MRD-negative status, peripheral blood counts recovered meeting CR criteria; in summary—patient started with MRD+ CRi and converted to MRD−CR. Another subject #66001-027 enrolled with 4% at baseline and after cycle 1 had 1% blasts; while MRD remained positive, this patient experienced 75% decrease in blast counts and proceeded to transplant. Only one patient #66001-029 in this cohort experienced disease progression.

Hence, applying a two-step dosage regimen comprising three different dosages of at least 30 μ/d as initial dose followed by a dose of at least 240 μg/d followed of a target dose of at least 600 μg/d may effectively convert an AML patient of MRD+ status to MRD− status and, thus, reduce the patients risk of a future disease progression.

### Example 4

The objectives of this study was to characterize the clinical safety of CD33×CD3 bispecific construct (as exemplified by SEQ ID NO 104) in MRD+ AML patients using the data from phase I dose-escalation study (NCT02520427). Patients were screened for at each dosage level of the respective treatment cycle for occurrence of CRS and in case of occurrence the event was graded according to generally accepted standards at the time when the clinical study has started (Lee et al., Blood 2014 Jul. 10; 124(2):188-95. doi: 10.1182/blood-2014-05-552729.). Subjects were pre-treated with 8 mg IV dexamethasone within 1 hour prior to the initial dose step of the CD33×CD3 bispecific construct (as exemplified by SEQ ID NO 104). Then, subjects were treated with a starting dose of 30 μg/d for 2 days, then with a dose of 240 μg/d for 5 days and then with a target dose of 600, 720, 840, 960, and 1600 μg/d for 21 days.

TABLE 7
Safety in terms of CRS occurrence and
grade in subjects treated for MRD AML

| Subject | 30 mcg/day | 240 mcg/day | 600 mcg/day |
|---|---|---|---|
| 66003-044 | G1 ~7 hrs | G1 ~31 hrs | G2 ~13 hrs |
| 66001-027 | — | G1, 15 hrs | — |
| 66001-029 | G1 24 hrs | — | — |

As safety result for the present study, Subject 66003-044 completed one CD33×CD3 bispecific construct (as exemplified by SEQ ID NO 104) cycle with no interruptions and no ICU transfers. The subject experienced the following key AEs: Grade 2 rash at 240 μg/day dose; Grade 1 CRS at 30 μg/day dose and 240 μg/day dose, Grade 2 CRS at 600 μg/day dose

Subject 66001-027 completed one CD33×CD3 bispecific construct (as exemplified by SEQ ID NO 104) cycle with no interruptions and no ICU transfers. The subject experienced the following key AEs: Grade 1 CRS and Grade 2 rash at 240 μg/day dose Subject 66001-029 completed one CD33×CD3 bispecific construct (as exemplified by SEQ ID NO 104) cycle with no interruptions and no ICU transfers. The subject experienced the following key AEs: Grade 1 CRS at 30 μg/day dose, Grade 2 rash at 240 μg/day dose.

In summary, the first dosage is safe as no patient with dose limiting toxicity (DLT) evaluable data has experiences CRS exceeding grade 1. The second dosage, i.e. after the first step, is tolerable as two patients experienced grade 1 CRS but none exceeded grade 1, and the target dose, i.e. after the second step, is considered safe as two patients did not experience CRS at all and one patient had grade 2 CRS below 48 h and had not exceeded grade 2. Hence, the safety of the CD33×CD3 bispecific construct (as exemplified by SEQ ID NO 104) is considered good for use in the treatment of MRD AML give n the surprisingly large second dosage step.

### Example 5

The objectives of this study was to characterize the clinical efficacy of CD33×CD3 bispecific construct (as exemplified by SEQ ID NO 104) in MDS patients using the data from phase I dose-escalation study (NCT02520427). Patients were screened for blast percentage at baseline and after the respective treatment cycle at baseline and after the respective treatment cycle. As a preferred dosage regimen, MSD patients receive cycle 2 after cycle 1 has been completed, i.e. without any infusion-free interval resulting in a duration of treatment of 56 days non-stop. An MDS patient in cohort 1 who had 12% blasts at baseline, showed 10% after cycle 1, i.e. no response, but 0% blasts after cycle 2. Hence, the CD33×CD3 bispecific construct (as exemplified by SEQ ID NO: 104) is effective for use in the treatment of MDS applying a dosage regimen as described herein.

| SEQ | | | |
|---|---|---|---|
| ID | | | |
| NO. | DESIGNATION | SOURCE | SEQUENCE |
| 1 | Human CD3e | Human | QDGNEEMGGITQTPYKVSISGTTVILTCPQYPGSEILWQHNDKNIGGDEDDKNIGSDEDHLSLKE |
| | extracellular domain | | FSELEQSGYYVCYPRGSKPEDANFYLYLRARVCENCMEMD |
| 2 | Human CD3e 1-27 | Human | QDGNEEMGGITQTPYKVSISGTTVILT |
| 3 | Callithrix jacchus | Callithrix | QDGNEEMGDTTQNPYKVSISGTTVTLTCPRYDGHEIKWLVNSQNKEGHEDHLLLEDFSEMEQS |
| | CD3e extracellular | jacchus | GYYACLSKETPAEEASHYLYLKARVCENCVEVD |
| | domain | | |
| 4 | Callithrix jacchus | Callithrix | QDGNEEMGDTTQNPYKVSISGTTVTLT |
| | CD3e 1-27 | jacchus | |
| 5 | Saguinus oedipus | Saguinus | QDGNEEMGDTTQNPYKVSISGTTVTLTCPRYDGHEIKWLVNSQNKEGHEDHLLLEDFSEMEQS |
| | CD3e extracellular | Oedipus | GYYACLSKETPAEEASHYLYLKARVCENCVEVD |
| | domain | | |
| 6 | Saguinus oedipus | Saguinus | QDGNEEMGDTTQNPYKVSISGTTVTLT |
| | CD3e 1-27 | Oedipus | |
| 7 | Saimiri sciureus CD3e | Saimiri | QDGNEEIGDTTQNPYKVSISGTTVTLTCPRYDGQEIKWLVNDQNKEGHEDHLLLEDFSEMEQSG |
| | extracellular domain | sciureus | YYACLSKETPTEEASHYLYLKARVCENCVEVD |
| 8 | Saimiri sciureus CD3e | Saimiri | QDGNEEIGDTTQNPYKVSISGTTVTLT |
| | 1-27 | sciureus | |
| 9 | CD33 VH of AH3 | Artificial | QVQLVQSGAEVKKPGESVKVSCKASGYTFTNYGMNWVRQAPGQGLEWMGWINTYTGEPTYA |
| | | | DDFKGRVTMSSDTSTSTAYLEINSLRSDDTAIYYCARWSWSDGYYVYFDYWGQGTTVTVSS |
| 10 | CD33 HCDR1 of AH3 | Artificial | NYGMN |
| 11 | CD33 HCDR2 of AH3 | Artificial | WINTYTGEPTYADDFKG |
| 12 | CD33 HCDR3 of AH3 | Artificial | WSWSDGYYVYFDY |
| 13 | CD33 VL of AH3 | Artificial | DIVMTQSPDSLTVSLGERTTINCKSSQSVLDSSKNKNSLAWYQQKPGQPPKLLLSWASTRESGIP |
| | | | DRFSGSGSGTDFTLTIDSLQPEDSATYYCQQSAHFPITFGQGTRLEIK |
| 14 | CD33 LCDR1 of AH3 | Artificial | KSSQSVLDSSKNKNSLA |
| 15 | CD33 LCDR2 of AH3 | Artificial | WASTRES |
| 16 | CD33 LCDR3 of AH3 | Artificial | QQSAHFPIT |
| 17 | CD33 CD33 HL of | Artificial | QVQLVQSGAEVKKPGESVKVSCKASGYTFTNYGMNWVRQAPGQGLEWMGWINTYTGEPTYA |
| | AH3 | | DDFKGRVTMSSDTSTSTAYLEINSLRSDDTAIYYCARWSWSDGYYVYFDYWGQGTTVTVSSGG |
| | | | GGSGGGGSGGGGSDIVMTQSPDSLTVSLGERTTINCKSSQSVLDSSKNKNSLAWYQQKPGQPPK |
| | | | LLLSWASTRESGIPDRFSGSGSGTDFTLTIDSLQPEDSATYYCQQSAHFPITFGQGTRLEIK |
| 18 | CD33 AH3 HL × H2C | Artificial | QVQLVQSGAEVKKPGESVKVSCKASGYTFTNYGMNWVRQAPGQGLEWMGWINTYTGEPTYA |
| | HL | | DDFKGRVTMSSDTSTSTAYLEINSLRSDDTAIYYCARWSWSDGYYVYFDYWGQGTTVTVSSGG |
| | | | GGSGGGGSGGGGSDIVMTQSPDSLTVSLGERTTINCKSSQSVLDSSKNKNSLAWYQQKPGQPPK |
| | | | LLLSWASTRESGIPDRFSGSGSGTDFTLTIDSLQPEDSATYYCQQSAHFPITFGQGTRLEIKSGGG |
| | | | GSEVQLVESGGGLVQPGGSLKLSCAASGFTFNKYAMNWVRQAPGKGLEWVARIRSKYNNYAT |
| | | | YYADSVKDRFTISRDDSKNTAYLQMNNLKTEDTAVYYCVRHGNFGNSYISYWAYWGQGTLVT |
| | | | VSSGGGGSGGGGSGGGGSQTVVTQEPSLTVSPGGTVTLTCGSSTGAVTSGYYPNWVQQKPGQA |
| | | | PRGLIGGTKFLAPGTPARFSGSLLGGKAALTLSGVQPEDEAEYYCALWYSNRWVFGGGTKLTV |
| | | | L |
| 19 | CD33 AH3 HL × | Artificial | QVQLVQSGAEVKKPGESVKVSCKASGYTFTNYGMNWVRQAPGQGLEWMGWINTYTGEPTYA |
| | F12Q HL | | DDFKGRVTMSSDTSTSTAYLEINSLRSDDTAIYYCARWSWSDGYYVYFDYWGQGTTVTVSSGG |
| | | | GGSGGGGSGGGGSDIVMTQSPDSLTVSLGERTTINCKSSQSVLDSSKNKNSLAWYQQKPGQPPK |
| | | | LLLSWASTRESGIPDRFSGSGSGTDFTLTIDSLQPEDSATYYCQQSAHFPITFGQGTRLEIKSGGG |
| | | | GSEVQLVESGGGLVQPGGSLKLSCAASGFTFNSYAMNWVRQAPGKGLEWVARIRSKYNNYAT |
| | | | YYADSVKGRFTISRDDSKNTAYLQMNNLKTEDTAVYYCVRHGNFGNSYVSWWAYWGQGTLV |
| | | | TVSSGGGGSGGGGSGGGGSQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLWYSNRWVFGGGTKLT |
| | | | VL |
| 20 | CD33 AH3 HL × I2C | Artificial | QVQLVQSGAEVKKPGESVKVSCKASGYTFTNYGMNWVRQAPGQGLEWMGWINTYTGEPTYA |
| | HL | | DDFKGRVTMSSDTSTSTAYLEINSLRSDDTAIYYCARWSWSDGYYVYFDYWGQGTTVTVSSGG |
| | | | GGSGGGGSGGGGSDIVMTQSPDSLTVSLGERTTINCKSSQSVLDSSKNKNSLAWYQQKPGQPPK |
| | | | LLLSWASTRESGIPDRFSGSGSGTDFTLTIDSLQPEDSATYYCQQSAHFPITFGQGTRLEIKSGGG |
| | | | GSEVQLVESGGGLVQPGGSLKLSCAASGFTFNKYAMNWVRQAPGKGLEWVARIRSKYNNYAT |
| | | | YYADSVKDRFTISRDDSKNTAYLQMNNLKTEDTAVYYCVRHGNFGNSYISYWAYWGQGTLVT |
| | | | VSSGGGGSGGGGSGGGGSQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWYSNRWVFGGGTKLTV |
| | | | L |
| 21 | CD33 VH of AF5 | Artificial | QVQLVQSGAEVKKPGASVKVSCKASGYTFTNYGMNWVKQAPGQGLKWMGWINTYTGEPTY |
| | | | ADDFKGRVTMTSDTSTSTAYLELHNLRSDDTAVYYCARWSWSDGYYVYFDYWGQGTTVTVS |
| | | | S |
| 22 | CD33 HCDR1 of AF5 | Artificial | NYGMN |
| 23 | CD33 HCDR2 of AF5 | Artificial | WINTYTGEPTYADDFKG |
| 24 | CD33 HCDR3 of AF5 | artificial | WSWSDGYYVYFDY |
| 25 | CD33 VL of AF5 | artificial | DIVMTQSPDSLTVSLGERTTINCKSSQSVLDSSKNKNSLAWYQQKPGQPPKLLLSWASTRESGIP |
| | | | DRFSGSGSGTDFTLTIDSLQPEDSATYYCQQSAHFPITFGQGTRLEIK |
| 26 | CD33 LCDR1 of AF5 | artificial | KSSQSVLDSSKNKNSLA |
| 27 | CD33 LCDR2 of AF5 | artificial | WASTRES |
| 28 | CD33 LCDR3 of AF5 | artificial | QQSAHFPIT |
| 29 | CD33HL of AF5 | artificial | QVQLVQSGAEVKKPGASVKVSCKASGYTFTNYGMNWVKQAPGQGLKWMGWINTYTGEPTY |
| | | | ADDFKGRVTMTSDTSTSTAYLELHNLRSDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIK |
| 30 | CD33 AF5 HL × H2C | artificial | QVQLVQSGAEVKKPGASVKVSCKASGYTFTNYGMNWVKQAPGQGLKWMGWINTYTGEPTY |
| | HL | | ADDFKGRVTMTSDTSTSTAYLELHNLRSDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQG |
| | | | TLVTVSSGGGGSGGGGSGGGGSQTVVTQEPSLTVSPGGTVTLTCGSSTGAVTSGYYPNWVQQK |
| | | | PGQAPRGLIGGTKFLAPGTPARFSGSLLGGKAALTLSGVQPEDEAEYYCALWYSNRWVFGGGT |
| | | | KLTVL |
| 31 | CD33 AF5 HL × F12Q | artificial | QVQLVQSGAEVKKPGASVKVSCKASGYTFTNYGMNWVKQAPGQGLKWMGWINTYTGEPTY |
| | HL | | ADDFKGRVTMTSDTSTSTAYLELHNLRSDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIKS |
| | | | GGGGSEVQLVESGGGLVQPGGSLKLSCAASGFTFNSYAMNWVRQAPGKGLEWVARIRSKYNN |
| | | | YATYYADSVKGRFTISRDDSKNTAYLQMNNLKTEDTAVYYCVRHGNFGNSYVSWWAYWGQ |
| | | | GTLVTVSSGGGGSGGGGSGGGGSQTVVTQEPSLTVSPGGTVTLTCGSSTGAVTSGNYPNWVQQ |
| | | | KPGQAPRGLIGGTKFLAPGTPARFSGSLLGGKAALTLSGVQPEDEAEYYCVLWYSNRWVFGGG |
| | | | TKLTVL |
| 32 | CD33 AF5 HL × I2C | artificial | QVQLVQSGAEVKKPGASVKVSCKASGYTFTNYGMNWVKQAPGQGLKWMGWINTYTGEPTY |
| | HL | | ADDFKGRVTMTSDTSTSTAYLELHNLRSDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQG |
| | | | TLVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGGT |
| | | | KLTVL |
| 33 | CD33 VH of AC8 | artificial | QVQLVQSGAEVKKPGESVKVSCKASGYTFTNYGMNWVKQAPGQGLKWMGWINTYTGEPTY |
| | | | ADDFKGRVTMTTDTSTSTAYMEIRNLRNDDTAVYYCARWSWSDGYYVYFDYWGQGTTVTVS |
| | | | S |
| 34 | CD33 HCDR1 of AC8 | artificial | NYGMN |
| 35 | CD33 HCDR2 of AC8 | artificial | WINTYTGEPTYADDFKG |
| 36 | CD33 HCDR3 of AC8 | artificial | WSWSDGYYVYFDY |
| 37 | CD33 VL of AC8 | artificial | DIVMTQSPDSLTVSLGERTTINCKSSQSVLDSSKNKNSLAWYQQKPGQPPKLLLSWASTRESGIP |
| | | | DRFSGSGSGTDFTLTIDSLQPEDSATYYCQQSAHFPITFGQGTRLEIK |
| 38 | CD33 LCDR1 of AC8 | artificial | KSSQSVLDSSKNKNSLA |
| 39 | CD33 LCDR2 of AC8 | artificial | WASTRES |
| 40 | CD33 LCDR3 of AC8 | artificial | QQSAHFPIT |
| 41 | CD33 HL of AC8 | artificial | QVQLVQSGAEVKKPGESVKVSCKASGYTFTNYGMNWVKQAPGQGLKWMGWINTYTGEPTY |
| | | | ADDFKGRVTMTTDTSTSTAYMEIRNLRNDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIK |
| 42 | CD33 AC8 HL × H2C | artificial | QVQLVQSGAEVKKPGESVKVSCKASGYTFTNYGMNWVKQAPGQGLKWMGWINTYTGEPTY |
| | HL | | ADDFKGRVTMTTDTSTSTAYMEIRNLRNDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQG |
| | | | TLVTVSSGGGGSGGGGSGGGGSQTVVTQEPSLTVSPGGTVTLTCGSSTGAVTSGYYPNWVQQK |
| | | | PGQAPRGLIGGTKFLAPGTPARFSGSLLGGKAALTLSGVQPEDEAEYYCALWYSNRWVFGGGT |
| | | | KLTVL |
| 43 | CD33 AC8 HL × F12Q | artificial | QVQLVQSGAEVKKPGESVKVSCKASGYTFTNYGMNWVKQAPGQGLKWMGWINTYTGEPTY |
| | HL | | ADDFKGRVTMTTDTSTSTAYMEIRNLRNDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIKS |
| | | | GGGGSEVQLVESGGGLVQPGGSLKLSCAASGFTFNSYAMNWVRQAPGKGLEWVARIRSKYNN |
| | | | YATYYADSVKGRFTISRDDSKNTAYLQMNNLKTEDTAVYYCVRHGNFGNSYVSWWAYWGQ |
| | | | GTLVTVSSGGGGSGGGGSGGGGSQTVVTQEPSLTVSPGGTVTLTCGSSTGAVTSGNYPNWVQQ |
| | | | KPGQAPRGLIGGTKFLAPGTPARFSGSLLGGKAALTLSGVQPEDEAEYYCVLWYSNRWVFGGG |
| | | | TKLTVL |
| 44 | CD33 AC8 HL × I2C | artificial | QVQLVQSGAEVKKPGESVKVSCKASGYTFTNYGMNWVKQAPGQGLKWMGWINTYTGEPTY |
| | HL | | ADDFKGRVTMTTDTSTSTAYMEIRNLRNDDTAVYYCARWSWSDGYYVYFDYWGQGTTVTVS |
| | | | SGGGGSGGGGSGGGGSDIVMTQSPDSLTVSLGERTTINCKSSQSVLDSSKNKNSLAWYQQKPG |
| | | | QPPKLLLSWASTRESGIPDRFSGSGSGTDFTLTIDSLQPEDSATYYCQQSAHFPITFGQGTRLEIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQG |
| | | | TLVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGGT |
| | | | KLTVL |
| 45 | CD33 VH of AH11 | artificial | QVQLVQSGAEVKKPGESVKVSCKASGYTFTNYGMNWVKQAPGQGLKWMGWINTYTGEPTY |
| | | | ADDFKGRVTMTSDTSTSTAYMEISSLRSDDTAVYYCARWSWSDGYYVYFDYWGQGTTVTVSS |
| 46 | CD33 HCDR1 of | artificial | NYGMN |
| | AH11 | | |
| 47 | CD33 HCDR2 of | artificial | WINTYTGEPTYADDFKG |
| | AH11 | | |
| 48 | HCDR3 | artificial | WSWSDGYYVYFDY |
| 49 | CD33 VL of AH11 | artificial | DIVMTQSPDSLTVSLGERTTINCKSSQSVLDSSKNKNSLAWYQQKPGQPPKLLLSWASTRESGIP |
| | | | DRFSGSGSGTDFTLTIDSLQPEDSATYYCQQSAHFPITFGQGTRLEIK |
| 50 | CD33 LCDR1 of | artificial | KSSQSVLDSSKNKNSLA |
| | AH11 | | |
| 51 | CD33 LCDR2 of | artificial | WASTRES |
| | AH11 | | |
| 52 | CD33LCDR3 of AH11 | artificial | QQSAHFPIT |
| 53 | CD33 HL of AH11 | artificial | QVQLVQSGAEVKKPGESVKVSCKASGYTFTNYGMNWVKQAPGQGLKWMGWINTYTGEPTY |
| | | | ADDFKGRVTMTSDTSTSTAYMEISSLRSDDTAVYYCARWSWSDGYYVYFDYWGQGTTVTVSS |
| | | | GGGGSGGGGSGGGGSDIVMTQSPDSLTVSLGERTTINCKSSQSVLDSSKNKNSLAWYQQKPGQ |
| | | | PPKLLLSWASTRESGIPDRFSGSGSGTDFTLTIDSLQPEDSATYYCQQSAHFPITFGQGTRLEIK |
| 54 | CD33 AH11 HL × | artificial | QVQLVQSGAEVKKPGESVKVSCKASGYTFTNYGMNWVKQAPGQGLKWMGWINTYTGEPTY |
| | H2C HL | | ADDFKGRVTMTSDTSTSTAYMEISSLRSDDTAVYYCARWSWSDGYYVYFDYWGQGTTVTVSS |
| | | | GGGGSGGGGSGGGGSDIVMTQSPDSLTVSLGERTTINCKSSQSVLDSSKNKNSLAWYQQKPGQ |
| | | | PPKLLLSWASTRESGIPDRFSGSGSGTDFTLTIDSLQPEDSATYYCQQSAHFPITFGQGTRLEIKSG |
| | | | GGGSEVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWGQGTL |
| | | | VTVSSGGGGSGGGGSGGGGSQTVVTQEPSLTVSPGGTVTLTCGSSTGAVTSGYYPNWVQQKPG |
| | | | QAPRGLIGGTKFLAPGTPARFSGSLLGGKAALTLSGVQPEDEAEYYCALWYSNRWVFGGGTKL |
| | | | TVL |
| 55 | CD33 AH11 HL × | artificial | QVQLVQSGAEVKKPGESVKVSCKASGYTFTNYGMNWVKQAPGQGLKWMGWINTYTGEPTY |
| | F12Q HL | | ADDFKGRVTMTSDTSTSTAYMEISSLRSDDTAVYYCARWSWSDGYYVYFDYWGQGTTVTVSS |
| | | | GGGGSGGGGSGGGGSDIVMTQSPDSLTVSLGERTTINCKSSQSVLDSSKNKNSLAWYQQKPGQ |
| | | | PPKLLLSWASTRESGIPDRFSGSGSGTDFTLTIDSLQPEDSATYYCQQSAHFPITFGQGTRLEIKSG |
| | | | GGGSEVQLVESGGGLVQPGGSLKLSCAASGFTFNSYAMNWVRQAPGKGLEWVARIRSKYNNY |
| | | | ATYYADSVKGRFTISRDDSKNTAYLQMNNLKTEDTAVYYCVRHGNFGNSYVSWWAYWGQGT |
| | | | LVTVSSGGGGSGGGGSGGGGSQTVVTQEPSLTVSPGGTVTLTCGSSTGAVTSGNYPNWVQQKP |
| | | | GQAPRGLIGGTKFLAPGTPARFSGSLLGGKAALTLSGVQPEDEAEYYCVLWYSNRWVFGGGTK |
| | | | LTVL |
| 56 | CD33 AH11 HL × I2C | artificial | QVQLVQSGAEVKKPGESVKVSCKASGYTFTNYGMNWVKQAPGQGLKWMGWINTYTGEPTY |
| | HL | | ADDFKGRVTMTSDTSTSTAYMEISSLRSDDTAVYYCARWSWSDGYYVYFDYWGQGTTVTVSS |
| | | | GGGGSGGGGSGGGGSDIVMTQSPDSLTVSLGERTTINCKSSQSVLDSSKNKNSLAWYQQKPGQ |
| | | | PPKLLLSWASTRESGIPDRFSGSGSGTDFTLTIDSLQPEDSATYYCQQSAHFPITFGQGTRLEIKSG |
| | | | GGGSEVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWGQGTL |
| | | | VTVSSGGGGSGGGGSGGGGSQTVVTQEPSLTVSPGGTVTLTCGSSTGAVTSGNYPNWVQQKPG |
| | | | QAPRGLIGGTKFLAPGTPARFSGSLLGGKAALTLSGVQPEDEAEYYCVLWYSNRWVFGGGTKL |
| | | | TVL |
| 57 | CD33 VH of B3 | artificial | QVQLVQSGAEVKKPGESVKVSCKASGYTFTNYGMNWVKQAPGQGLEWMGWINTYTGETNY |
| | | | ADKFQGRVTFTSDTSTSTAYMELRNLKSDDTAVYYCARWSWSDGYYVYFDYWGQGTTVTVS |
| | | | S |
| 58 | CD33 HCDR1 of B3 | artificial | NYGMN |
| 59 | CD33 HCDR2 of B3 | artificial | WINTYTGETNYADKFQG |
| 60 | CD33 HCDR3 of B3 | artificial | WSWSDGYYVYFDY |
| 61 | CD33 VL of B3 | artificial | DIVMTQSPDSMTVSLGERTTINCKSSQSVLDSSTNKNSLAWYQQKPGQPPKLLLSWASTRESGIP |
| | | | DRFSGSGSGTDFTLTIDSLQPEDSATYYCQQSAHFPITFGQGTRLDIK |
| 62 | CD33 LCDR1 of B3 | artificial | KSSQSVLDSSTNKNSLA |
| 63 | CD33 LCDR2 of B3 | artificial | WASTRE |
| 64 | CD33LCDR3 of B3 | artificial | QQSAHFPIT |
| 65 | CD33 HL of B3 | artificial | QVQLVQSGAEVKKPGESVKVSCKASGYTFTNYGMNWVKQAPGQGLEWMGWINTYTGETNY |
| | | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSMTVSLGERTTINCKSSQSVLDSSTNKNSLAWYQQKPG |
| | | | QPPKLLLSWASTRESGIPDRFSGSGSGTDFTLTIDSLQPEDSATYYCQQSAHFPITFGQGTRLDIK |
| 66 | CD33 B3 HL × H2C | artificial | QVQLVQSGAEVKKPGESVKVSCKASGYTFTNYGMNWVKQAPGQGLEWMGWINTYTGETNY |
| | HL | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSMTVSLGERTTINCKSSQSVLDSSTNKNSLAWYQQKPG |
| | | | QPPKLLLSWASTRESGIPDRFSGSGSGTDFTLTIDSLQPEDSATYYCQQSAHFPITFGQGTRLDIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQG |
| | | | TLVTVSSGGGGSGGGGSGGGGSQTVVTQEPSLTVSPGGTVTLTCGSSTGAVTSGYYPNWVQQK |
| | | | PGQAPRGLIGGTKFLAPGTPARFSGSLLGGKAALTLSGVQPEDEAEYYCALWYSNRWVFGGGT |
| | | | KLTVL |
| 67 | CD33 B3 HL × F12Q | artificial | QVQLVQSGAEVKKPGESVKVSCKASGYTFTNYGMNWVKQAPGQGLEWMGWINTYTGETNY |
| | HL | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSMTVSLGERTTINCKSSQSVLDSSTNKNSLAWYQQKPG |
| | | | QPPKLLLSWASTRESGIPDRFSGSGSGTDFTLTIDSLQPEDSATYYCQQSAHFPITFGQGTRLDIKS |
| | | | GGGGSEVQLVESGGGLVQPGGSLKLSCAASGFTFNSYAMNWVRQAPGKGLEWVARIRSKYNN |
| | | | YATYYADSVKGRFTISRDDSKNTAYLQMNNLKTEDTAVYYCVRHGNFGNSYVSWWAYWGQ |
| | | | GTLVTVSSGGGGSGGGGSGGGGSQTVVTQEPSLTVSPGGTVTLTCGSSTGAVTSGNYPNWVQQ |
| | | | KPGQAPRGLIGGTKFLAPGTPARFSGSLLGGKAALTLSGVQPEDEAEYYCVLWYSNRWVFGGG |
| | | | TKLTVL |
| 68 | CD33 B3 HL × I2C | artificial | QVQLVQSGAEVKKPGESVKVSCKASGYTFTNYGMNWVKQAPGQGLEWMGWINTYTGETNY |
| | HL | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSMTVSLGERTTINCKSSQSVLDSSTNKNSLAWYQQKPG |
| | | | QPPKLLLSWASTRESGIPDRFSGSGSGTDFTLTIDSLQPEDSATYYCQQSAHFPITFGQGTRLDIKS |
| | | | GGGGSEVQLVESGGGLVQPGGSLKLSCAASGFTFNKYAMNWVRQAPGKGLEWVARIRSKYNN |
| | | | YATYYADSVKDRFTISRDDSKNTAYLQMNNLKTEDTAVYYCVRHGNFGNSYISYWAYWGQG |
| | | | TLVTVSSGGGGSGGGGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYCVLWYSNRWVFGGGT |
| | | | KLTVL |
| 69 | CD33 VH of F2 | artificial | QVQLVQSGAEVKKPGESVKVSCKASGYTFTNYGMNWVKQAPGQGLEWMGWINTYTGETNY |
| | | | ADKFQGRVTFTSDTSTSTAYMELRNLKSDDTAVYYCARWSWSDGYYVYFDYWGQGTTVTVS |
| | | | S |
| 70 | CD33 HCDR1 of F2 | artificial | NYGMN |
| 71 | CD33 HCDR2 of F2 | artificial | WINTYTGETNYADKFQG |
| 72 | CD33 HCDR3 of F2 | artificial | WSWSDGYYVYFDY |
| 73 | CD33 VL of F2 | artificial | DIVMTQSPDSLSVSLGERTTINCKSSQSVLDSSTNKNSLAWYQQKPGQPPKLLLSWASTRESGIP |
| | | | DRFSGSGSGTDFTLTIDSLQPEDSATYYCQQSAHFPITFGQGTRLEIK |
| 74 | CD33 LCDR1 of F2 | artificial | KSSQSVLDSSTNKNSLA |
| 75 | CD33 LCDR2 of F2 | artificial | WASTRES |
| 76 | CD33 LCDR3 of F2 | artificial | QQSAHFPIT |
| 77 | CD33 HL of F2 | artificial | QVQLVQSGAEVKKPGESVKVSCKASGYTFTNYGMNWVKQAPGQGLEWMGWINTYTGETNY |
| | | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSLSVSLGERTTINCKSSQSVLDSSTNKNSLAWYQQKPGQ |
| | | | PPKLLLSWASTRESGIPDRFSGSGSGTDFTLTIDSLQPEDSATYYCQQSAHFPITFGQGTRLEIK |
| 78 | CD33 F2 HL × H2C | artificial | QVQLVQSGAEVKKPGESVKVSCKASGYTFTNYGMNWVKQAPGQGLEWMGWINTYTGETNY |
| | HL | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSLSVSLGERTTINCKSSQSVLDSSTNKNSLAWYQQKPGQ |
| | | | PPKLLLSWASTRESGIPDRFSGSGSGTDFTLTIDSLQPEDSATYYCQQSAHFPITFGQGTRLEIKSG |
| | | | GGGSEVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWGQGTL |
| | | | VTVSSGGGGSGGGGSGGGGSQTVVTQEPSLTVSPGGTVTLTCGSSTGAVTSGYYPNWVQQKPG |
| | | | QAPRGLIGGTKFLAPGTPARFSGSLLGGKAALTLSGVQPEDEAEYYCALWYSNRWVFGGGTKL |
| | | | TVL |
| 79 | CD33 F2 HL × F12Q | artificial | QVQLVQSGAEVKKPGESVKVSCKASGYTFTNYGMNWVKQAPGQGLEWMGWINTYTGETNY |
| | HL | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSLSVSLGERTTINCKSSQSVLDSSTNKNSLAWYQQKPGQ |
| | | | PPKLLLSWASTRESGIPDRFSGSGSGTDFTLTIDSLQPEDSATYYCQQSAHFPITFGQGTRLEIKSG |
| | | | GGGSEVQLVESGGGLVQPGGSLKLSCAASGFTFNSYAMNWVRQAPGKGLEWVARIRSKYNNY |
| | | | ATYYADSVKGRFTISRDDSKNTAYLQMNNLKTEDTAVYYCVRHGNFGNSYVSWWAYWGQGT |
| | | | LVTVSSGGGGSGGGGSGGGGSQTVVTQEPSLTVSPGGTVTLTCGSSTGAVTSGNYPNWVQQKP |
| | | | GQAPRGLIGGTKFLAPGTPARFSGSLLGGKAALTLSGVQPEDEAEYYCVLWYSNRWVFGGGTK |
| | | | LTVL |
| 80 | CD33 F2 HL × I2C HL | artificial | QVQLVQSGAEVKKPGESVKVSCKASGYTFTNYGMNWVKQAPGQGLEWMGWINTYTGETNY |
| | | | ADKFQGRVTFTSDTSTSTAYMELRNLKSDDTAVYYCARWSWSDGYYVYFDYWGQGTTVTVS |
| | | | SGGGGSGGGGSGGGGSDIVMTQSPDSLSVSLGERTTINCKSSQSVLDSSTNKNSLAWYQQKPGQ |
| | | | PPKLLLSWASTRESGIPDRFSGSGSGTDFTLTIDSLQPEDSATYYCQQSAHFPITFGQGTRLEIKSG |
| | | | GGGSEVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWGQGTL |
| | | | VTVSSGGGGSGGGGSGGGGSQTVVTQEPSLTVSPGGTVTLTCGSSTGAVTSGNYPNWVQQKPG |
| | | | QAPRGLIGGTKFLAPGTPARFSGSLLGGKAALTLSGVQPEDEAEYYCVLWYSNRWVFGGGTKL |
| | | | TVL |
| 81 | CD33 VH of B10 | artificial | QVQLVQSGAEVKKPGESVKVSCKASGYTFTNYGMNWVKQAPGQGLEWMGWINTYTGEPTYA |
| | | | DKFQGRVTMTTDTSTSTAYMEIRNLRSDDTAVYYCARWSWSDGYYVYFDYWGQGTTVTVSS |
| 82 | CD33 HCDR1 of B10 | Artificial | NYGMN |
| 83 | CD33HCDR2 of B10 | artificial | WINTYTGEPTYADKFQG |
| 84 | CD33 HCDR3 of B10 | artificial | WSWSDGYYVYFDY |
| 85 | CD33 VL of B10 | artificial | DIVMTQSPDSLTVSLGERTTINCKSSQSVLDSSNNKNSLAWYQQKPGQPPKLLLSWASTRESGIP |
| | | | DRFSGSGSGTDFTLTIDGLQPEDSATYYCQQSAHFPITFGQGTRLEIK |
| 86 | CD33 LCDR1 of B10 | artificial | KSSQSVLDSSNNKNSLA |
| 87 | CD33 LCDR2 of B10 | artificial | WASTRES |
| 88 | CD33 LCDR3 of B10 | artificial | QQSAHFPIT |
| 89 | CD33 HL of B10 | artificial | QVQLVQSGAEVKKPGESVKVSCKASGYTFTNYGMNWVKQAPGQGLEWMGWINTYTGEPTYA |
| | | | DKFQGRVTMTTDTSTSTAYMEIRNLRSDDTAVYYCARWSWSDGYYVYFDYWGQGTTVTVSS |
| | | | GGGGSGGGGSGGGGSDIVMTQSPDSLTVSLGERTTINCKSSQSVLDSSNNKNSLAWYQQKPGQ |
| | | | PPKLLLSWASTRESGIPDRFSGSGSGTDFTLTIDGLQPEDSATYYCQQSAHFPITFGQGTRLEIK |
| 90 | CD33 B10 HL × H2C | artificial | QVQLVQSGAEVKKPGESVKVSCKASGYTFTNYGMNWVKQAPGQGLEWMGWINTYTGEPTYA |
| | HL | | DKFQGRVTMTTDTSTSTAYMEIRNLRSDDTAVYYCARWSWSDGYYVYFDYWGQGTTVTVSS |
| | | | GGGGSGGGGSGGGGSDIVMTQSPDSLTVSLGERTTINCKSSQSVLDSSNNKNSLAWYQQKPGQ |
| | | | PPKLLLSWASTRESGIPDRFSGSGSGTDFTLTIDGLQPEDSATYYCQQSAHFPITFGQGTRLEIKSG |
| | | | GGGSEVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWGQGTL |
| | | | VTVSSGGGGSGGGGSGGGGSQTVVTQEPSLTVSPGGTVTLTCGSSTGAVTSGYYPNWVQQKPG |
| | | | QAPRGLIGGTKFLAPGTPARFSGSLLGGKAALTLSGVQPEDEAEYYCALWYSNRWVFGGGTKL |
| | | | TVL |
| 91 | CD33 B10 HL × F12Q | artificial | QVQLVQSGAEVKKPGESVKVSCKASGYTFTNYGMNWVKQAPGQGLEWMGWINTYTGEPTYA |
| | HL | | DKFQGRVTMTTDTSTSTAYMEIRNLRSDDTAVYYCARWSWSDGYYVYFDYWGQGTTVTVSS |
| | | | GGGGSGGGGSGGGGSDIVMTQSPDSLTVSLGERTTINCKSSQSVLDSSNNKNSLAWYQQKPGQ |
| | | | PPKLLLSWASTRESGIPDRFSGSGSGTDFTLTIDGLQPEDSATYYCQQSAHFPITFGQGTRLEIKSG |
| | | | GGGSEVQLVESGGGLVQPGGSLKLSCAASGFTFNSYAMNWVRQAPGKGLEWVARIRSKYNNY |
| | | | ATYYADSVKGRFTISRDDSKNTAYLQMNNLKTEDTAVYYCVRHGNFGNSYVSWWAYWGQGT |
| | | | LVTVSSGGGGSGGGGSGGGGSQTVVTQEPSLTVSPGGTVTLTCGSSTGAVTSGNYPNWVQQKP |
| | | | GQAPRGLIGGTKFLAPGTPARFSGSLLGGKAALTLSGVQPEDEAEYYCVLWYSNRWVFGGGTK |
| | | | LTVL |
| 92 | CD33 B10 HL × I2C | artificial | QVQLVQSGAEVKKPGESVKVSCKASGYTFTNYGMNWVKQAPGQGLEWMGWINTYTGEPTYA |
| | HL | | DKFQGRVTMTTDTSTSTAYMEIRNLRSDDTAVYYCARWSWSDGYYVYFDYWGQGTTVTVSS |
| | | | GGGGSGGGGSGGGGSDIVMTQSPDSLTVSLGERTTINCKSSQSVLDSSNNKNSLAWYQQKPGQ |
| | | | PPKLLLSWASTRESGIPDRFSGSGSGTDFTLTIDGLQPEDSATYYCQQSAHFPITFGQGTRLEIKSG |
| | | | GGGSEVQLVESGGGLVQPGGSLKLSCAASGFTFNKYAMNWVRQAPGKGLEWVARIRSKYNNY |
| | | | ATYYADSVKDRFTISRDDSKNTAYLQMNNLKTEDTAVYYCVRHGNFGNSYISYWAYWGQGTL |
| | | | VTVSSGGGGSGGGGSGGGGSQTVVTQEPSLTVSPGGTVTLTCGSSTGAVTSGNYPNWVQQKPG |
| | | | QAPRGLIGGTKFLAPGTPARFSGSLLGGKAALTLSGVQPEDEAEYYCVLWYSNRWVFGGGTKL |
| | | | TVL |
| 93 | CD33 VH of E11 | artificial | QVQLVQSGAEVKKPGESVKVSCKASGYTFTNYGMNWVKQAPGQGLEWMGWINTYTGEPTYA |
| | | | DKFQGRVTMTTDTSTSTAYMEIRNLGGDDTAVYYCARWSWSDGYYVYFDYWGQGTSVTVSS |
| 94 | CD33 HCDR1 of E11 | artificial | NYGMN |
| 95 | CD33 HCDR2 of E11 | artificial | WINTYTGEPTYADKFQG |
| 96 | CD33 HCDR3 of E11 | artificial | WSWSDGYYVYFDY |
| 97 | CD33 VL of E11 | artificial | DIVMTQSPDSLTVSLGERTTINCKSSQSVLDSSTNKNSLAWYQQKPGQPPKLLLSWASTRESGIP |
| | | | DRFSGSGSGTDFTLTIDSPQPEDSATYYCQQSAHFPITFGQGTRLEIK |
| 98 | CD33 LCDR1 of E11 | artificial | KSSQSVLDSSTNKNSLA |
| 99 | CD33 LCDR2 of E11 | artificial | WASTRES |
| 100 | CD33 LCDR3 of E11 | artificial | QQSAHFPIT |
| 101 | CD33 HL of E11 | artificial | QVQLVQSGAEVKKPGESVKVSCKASGYTFTNYGMNWVKQAPGQGLEWMGWINTYTGEPTYA |
| | | | DKFQGRVTMTTDTSTSTAYMEIRNLGGDDTAVYYCARWSWSDGYYVYFDYWGQGTSVTVSS |
| | | | GGGGSGGGGSGGGGSDIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSAHFPITFGQGTRLEIK |
| 102 | CD33 E11 HL × H2C | artificial | QVQLVQSGAEVKKPGESVKVSCKASGYTFTNYGMNWVKQAPGQGLEWMGWINTYTGEPTYA |
| | HL | | DKFQGRVTMTTDTSTSTAYMEIRNLGGDDTAVYYCARWSWSDGYYVYFDYWGQGTSVTVSS |
| | | | GGGGSGGGGSGGGGSDIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSAHFPITFGQGTRLEIKSGG |
| | | | GGSEVQLVESGGGLVQPGGSLKLSCAASGFTFNKYAMNWVRQAPGKGLEWVARIRSKYNNYA |
| | | | TYYADSVKDRFTISRDDSKNTAYLQMNNLKTEDTAVYYCVRHGNFGNSYISYWAYWGQGTLV |
| | | | TVSSGGGGSGGGGSGGGGSQTVVTQEPSLTVSPGGTVTLTCGSSTGAVTSGYYPNWVQQKPGQ |
| | | | APRGLIGGTKFLAPGTPARFSGSLLGGKAALTLSGVQPEDEAEYYCALWYSNRWVFGGGTKLT |
| | | | VL |
| 103 | CD33 E11 HL × F12Q | artificial | QVQLVQSGAEVKKPGESVKVSCKASGYTFTNYGMNWVKQAPGQGLEWMGWINTYTGEPTYA |
| | HL | | DKFQGRVTMTTDTSTSTAYMEIRNLGGDDTAVYYCARWSWSDGYYVYFDYWGQGTSVTVSS |
| | | | GGGGSGGGGSGGGGSDIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSAHFPITFGQGTRLEIKSGG |
| | | | GGSEVQLVESGGGLVQPGGSLKLSCAASGFTFNSYAMNWVRQAPGKGLEWVARIRSKYNNYA |
| | | | TYYADSVKGRFTISRDDSKNTAYLQMNNLKTEDTAVYYCVRHGNFGNSYVSWWAYWGQGTL |
| | | | VTVSSGGGGSGGGGSGGGGSQTVVTQEPSLTVSPGGTVTLTCGSSTGAVTSGNYPNWVQQKPG |
| | | | QAPRGLIGGTKFLAPGTPARFSGSLLGGKAALTLSGVQPEDEAEYYCVLWYSNRWVFGGGTKL |
| | | | TVL |
| 104 | CD33 E11 HL × I2C | artificial | QVQLVQSGAEVKKPGESVKVSCKASGYTFTNYGMNWVKQAPGQGLEWMGWINTYTGEPTYA |
| | HL | | DKFQGRVTMTTDTSTSTAYMEIRNLGGDDTAVYYCARWSWSDGYYVYFDYWGQGTSVTVSS |
| | | | GGGGSGGGGSGGGGSDIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSAHFPITFGQGTRLEIKSGG |
| | | | GGSEVQLVESGGGLVQPGGSLKLSCAASGFTFNKYAMNWVRQAPGKGLEWVARIRSKYNNYA |
| | | | TYYADSVKDRFTISRDDSKNTAYLQMNNLKTEDTAVYYCVRHGNFGNSYISYWAYWGQGTLV |
| | | | TVSSGGGGSGGGGSGGGGSQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLWYSNRWVFGGGTKLT |
| | | | VL |
| 105 | CD33 UD H2C HL × | artificial | EVQLVESGGGLVQPGGSLKLSCAASGFTFNKYAMNWVRQAPGKGLEWVARIRSKYNNYATY |
| | AF5 HL | | YADSVKDRFTISRDDSKNTAYLQMNNLKTEDTAVYYCVRHGNFGNSYISYWAYWGQGTLVTV |
| | | | SSGGGGSGGGGSGGGGSQTVVTQEPSLTVSPGGTVTLTCGSSTGAVTSGYYPNWVQQKPGQAP |
| | | | RGLIGGTKFLAPGTPARFSGSLLGGKAALTLSGVQPEDEAEYYCALWYSNRWVFGGGTKLTVL |
| | | | SGGGGSQVQLVQSGAEVKKPGASVKVSCKASGYTFTNYGMNWVKQAPGQGLKWMGWINTY |
| | | | TGEPTYADDFKGRVTMTSDTSTSTAYLELHNLRSDDTAVYYCARWSWSDGYYVYFDYWGQG |
| | | | TTVTVSSGGGGSGGGGSGGGGSDIVMTQSPDSLTVSLGERTTINCKSSQSVLDSSKNKNSLAWY |
| | | | QQKPGQPPKLLLSWASTRESGIPDRFSGSGSGTDFTLTIDSLQPEDSATYYCQQSAHFPITFGQGT |
| | | | RLEIK |
| 106 | CD33 UD F12Q HL × | artificial | EVQLVESGGGLVQPGGSLKLSCAASGFTFNSYAMNWVRQAPGKGLEWVARIRSKYNNYATYY |
| | AF5 HL | | ADSVKGRFTISRDDSKNTAYLQMNNLKTEDTAVYYCVRHGNFGNSYVSWWAYWGQGTLVTV |
| | | | SSGGGGSGGGGSGGGGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTVL |
| | | | SGGGGSQVQLVQSGAEVKKPGASVKVSCKASGYTFTNYGMNWVKQAPGQGLKWMGWINTY |
| | | | TGEPTYADDFKGRVTMTSDTSTSTAYLELHNLRSDDTAVYYCARWSWSDGYYVYFDYWGQG |
| | | | TTVTVSSGGGGSGGGGSGGGGSDIVMTQSPDSLTVSLGERTTINCKSSQSVLDSSKNKNSLAWY |
| | | | QQKPGQPPKLLLSWASTRESGIPDRFSGSGSGTDFTLTIDSLQPEDSATYYCQQSAHFPITFGQGT |
| | | | RLEIK |
| 107 | CD33 UD I2C HL × | artificial | EVQLVESGGGLVQPGGSLKLSCAASGFTFNKYAMNWVRQAPGKGLEWVARIRSKYNNYATY |
| | AF5 HL | | YADSVKDRFTISRDDSKNTAYLQMNNLKTEDTAVYYCVRHGNFGNSYISYWAYWGQGTLVTV |
| | | | SSGGGGSGGGGSGGGGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYSNRWVFGGGTKLTVL |
| | | | SGGGGSQVQLVQSGAEVKKPGASVKVSCKASGYTFTNYGMNWVKQAPGQGLKWMGWINTY |
| | | | TGEPTYADDFKGRVTMTSDTSTSTAYLELHNLRSDDTAVYYCARWSWSDGYYVYFDYWGQG |
| | | | TTVTVSSGGGGSGGGGSGGGGSDIVMTQSPDSLTVSLGERTTINCKSSQSVLDSSKNKNSLAWY |
| | | | QQKPGQPPKLLLSWASTRESGIPDRFSGSGSGTDFTLTIDSLQPEDSATYYCQQSAHFPITFGQGT |
| | | | RLEIK |
| 108 | CD33 E11 HL × I2C | artificial | QVQLVQSGAEVKKPGESVKVSCKASGYTFTNYGMNWVKQAPGQGLEWMGWINTYTGEPTYA |
| | HL H6 | | DKFQGRVTMTTDTSTSTAYMEIRNLGGDDTAVYYCARWSWSDGYYVYFDYWGQGTSVTVSS |
| | | | GGGGSGGGGSGGGGSDIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSAHFPITFGQGTRLEIKSGG |
| | | | GGSEVQLVESGGGLVQPGGSLKLSCAASGFTFNKYAMNWVRQAPGKGLEWVARIRSKYNNYA |
| | | | TYYADSVKDRFTISRDDSKNTAYLQMNNLKTEDTAVYYCVRHGNFGNSYISYWAYWGQGTLV |
| | | | TVSSGGGGSGGGGSGGGGSQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLWYSNRWVFGGGTKLT |
| | | | VLHHHHHH |
| 109 | HALB | human | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGKKLVAASQAALGL |
| 110 | HALB7 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAG |
| | | | TFTFHADICTLSEKERQIKKQTALVELVKHKPKATKEQLKAAMDDFAAFVEKCCKADDKETCF |
| | | | AEEGKKLVAASQAALGL |
| 111 | HALB098 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGPKLVAASQAALGL |
| 112 | HALB114 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDDFAAFVEKCCKADDKETCF |
| | | | AEEGPHLVAASKAALGL |
| 113 | HALB254 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALGVDETYVPKEFNAE |
| | | | TFTFHADICTLSEKERQIKKQTALVELVKHKPKATKEQLKAVMDKFAAFVEKCCKADDKETCF |
| | | | AEEGPKLVAASQAALGL |
| 114 | HALB253 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDKFAAFVEKCCKADDKETCF |
| | | | AEEGPKLVAASQAALGL |
| 115 | HALB131 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDKFAAFVEKCCKADDKETCF |
| | | | AEEGPHLVAASQAALGL |
| 116 | HALB135 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGPHLVAASKAALGL |
| 117 | HALB133 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDKFAAFVEKCCKADDKETCF |
| | | | AEEGPKLVAASKAALGL |
| 118 | HALB234 | artificial | DAHKSEVAHRFKDLGEENFKALVLIAFAQYLQQCPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDDFAAFVEKCCKADDKETCF |
| | | | AEEGPKLVAASKAALGL |
| 119 | HALB C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGKKLVAASQAALGL |
| 120 | HALB7 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAG |
| | | | TFTFHADICTLSEKERQIKKQTALVELVKHKPKATKEQLKAAMDDFAAFVEKCCKADDKETCF |
| | | | AEEGKKLVAASQAALGL |
| 121 | HALB098 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGPKLVAASQAALGL |
| 122 | HALB114 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDDFAAFVEKCCKADDKETCF |
| | | | AEEGPHLVAASKAALGL |
| 123 | HALB254 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALGVDETYVPKEFNAE |
| | | | TFTFHADICTLSEKERQIKKQTALVELVKHKPKATKEQLKAVMDKFAAFVEKCCKADDKETCF |
| | | | AEEGPKLVAASQAALGL |
| 124 | HALB253 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDKFAAFVEKCCKADDKETCF |
| | | | AEEGPKLVAASQAALGL |
| 125 | HALB131 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDKFAAFVEKCCKADDKETCF |
| | | | AEEGPHLVAASQAALGL |
| 126 | HALB135 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGPHLVAASKAALGL |
| 127 | HALB133 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDKFAAFVEKCCKADDKETCF |
| | | | AEEGPKLVAASKAALGL |
| 128 | HALB234 C34S | artificial | DAHKSEVAHRFKDLGEENFKALVLIAFAQYLQQSPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDDFAAFVEKCCKADDKETCF |
| | | | AEEGPKLVAASKAALGL |
| 129 | HALB C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGKKLVAASQAALGL |
| 130 | HALB7 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAG |
| | | | TFTFHADICTLSEKERQIKKQTALVELVKHKPKATKEQLKAAMDDFAAFVEKCCKADDKETCF |
| | | | AEEGKKLVAASQAALGL |
| 131 | HALB098 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGPKLVAASQAALGL |
| 132 | HALB114 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDDFAAFVEKCCKADDKETCF |
| | | | AEEGPHLVAASKAALGL |
| 133 | HALB254 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALGVDETYVPKEFNAE |
| | | | TFTFHADICTLSEKERQIKKQTALVELVKHKPKATKEQLKAVMDKFAAFVEKCCKADDKETCF |
| | | | AEEGPKLVAASQAALGL |
| 134 | HALB253 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDKFAAFVEKCCKADDKETCF |
| | | | AEEGPKLVAASQAALGL |
| 135 | HALB131 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDKFAAFVEKCCKADDKETCF |
| | | | AEEGPHLVAASQAALGL |
| 136 | HALB135 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDDFAAFVEKCCKADDKETCF |
| | | | AEEGPHLVAASKAALGL |
| 137 | HALB133 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALEVDETYVPKEFNAE |
| | | | TFTFHADICTLSEKERQIKKQTALVELVKHKPKATKEQLKAVMDKFAAFVEKCCKADDKETCF |
| | | | AEEGPKLVAASKAALGL |
| 138 | HALB234 C34A | artificial | DAHKSEVAHRFKDLGEENFKALVLIAFAQYLQQAPFEDHVKLVNEVTEFAKTCVADESAENCD |
| | | | KSLHTLFGDKLCTVATLRETYGEMADCCAKQEPERNECFLQHKDDNPNLPRLVRPEVDVMCT |
| | | | AFHDNEETFLKKYLYEIARRHPYFYAPELLFFAKRYKAAFTECCQAADKAACLLPKLDELRDEG |
| | | | KASSAKQRLKCASLQKFGERAFKAWAVARLSQRFPKAEFAEVSKLVTDLTKVHTECCHGDLLE |
| | | | CADDRADLAKYICENQDSISSKLKECCEKPLLEKSHCIAEVENDEMPADLPSLAADFVESKDVC |
| | | | KNYAEAKDVFLGMFLYEYARRHPDYSVVLLLRLAKTYETTLEKCCAAADPHECYAKVFDEFK |
| | | | PLVEEPQNLIKQNCELFEQLGEYKFQNALLVRYTKKVPQVSTPTLVEVSRNLGKVGSKCCKHPE |
| | | | AKRMPCAEDYLSVVLNQLCVLHEKTPVSDRVTKCCTESLVNRRPCFSALDVDETYVPKEFNAE |
| | | | TFTFHADICTLSEKERQIKKQTALVELVKHKPKATKEQLKAVMDDFAAFVEKCCKADDKETCF |
| | | | AEEGPKLVAASKAALGL |
| 139 | Peptide linker | artificial | GGGG |
| 140 | Peptide linker | artificial | GGGGS |
| 141 | Peptide linker | artificial | GGGGQ |
| 142 | Peptide linker | artificial | PGGGGS |
| 143 | Peptide linker | artificial | PGGDGS |
| 144 | Peptide linker | artificial | SGGGGS |
| 145 | Peptide linker | artificial | GGGGSGGGS |
| 146 | Peptide linker | artificial | GGGGSGGGGS |
| 147 | Peptide linker | artificial | GGGGSGGGGSGGGGS |
| 148 | CDR-L1 of F6A | artificial | GSSTGAVTSGYYPN |
| 149 | CDR-L2 of F6A | artificial | GTKFLAP |
| 150 | CDR-L3 of F6A | artificial | ALWYSNRWV |
| 151 | CDR-H1 of F6A | artificial | IYAMN |
| 152 | CDR-H2 of F6A | artificial | RIRSKYNNYATYYADSVKS |
| 153 | CDR-H3 of F6A | artificial | HGNFGNSYVSFFAY |
| 154 | CDR-L1 of H2C | artificial | GSSTGAVTSGYYPN |
| 155 | CDR-L2 of H2C | artificial | GTKFLAP |
| 156 | CDR-L3 of H2C | artificial | ALWYSNRWV |
| 157 | CDR-H1 of H2C | artificial | KYAMN |
| 158 | CDR-H2 of H2C | artificial | RIRSKYNNYATYYADSVKD |
| 159 | CDR-H3 of H2C | artificial | HGNFGNSYISYWAY |
| 160 | CDR-L1 of H1E | artificial | GSSTGAVTSGYYPN |
| 161 | CDR-L2 of H1E | artificial | GTKFLAP |
| 162 | CDR-L3 of H1E | artificial | ALWYSNRWV |
| 163 | CDR-H1 of H1E | artificial | SYAMN |
| 164 | CDR-H2 of H1E | artificial | RIRSKYNNYATYYADSVKG |
| 165 | CDR-H3 of H1E | artificial | HGNFGNSYLSFWAY |
| 166 | CDR-L1 of G4H | artificial | GSSTGAVTSGYYPN |
| 167 | CDR-L2 of G4H | artificial | GTKFLAP |
| 168 | CDR-L3 of G4H | artificial | ALWYSNRWV |
| 169 | CDR-H1 of G4H | artificial | RYAMN |
| 170 | CDR-H2 of G4H | artificial | RIRSKYNNYATYYADSVKG |
| 171 | CDR-H3 of G4H | artificial | HGNFGNSYLSYFAY |
| 172 | CDR-L1 of A2J | artificial | RSSTGAVTSGYYPN |
| 173 | CDR-L2 of A2J | artificial | ATDMRPS |
| 174 | CDR-L3 of A2J | artificial | ALWYSNRWV |
| 175 | CDR-H1 of A2J | artificial | VYAMN |
| 176 | CDR-H2 of A2J | artificial | RIRSKYNNYATYYADSVKK |
| 177 | CDR-H3 of A2J | artificial | HGNFGNSYLSWWAY |
| 178 | CDR-L1 of E1L | artificial | GSSTGAVTSGYYPN |
| 179 | CDR-L2 of E1L | artificial | GTKFLAP |
| 180 | CDR-L3 of E1L | artificial | ALWYSNRWV |
| 181 | CDR-H1 of E1L | artificial | KYAMN |
| 182 | CDR-H2 of E1L | artificial | RIRSKYNNYATYYADSVKS |
| 183 | CDR-H3 of E1L | artificial | HGNFGNSYTSYYAY |
| 184 | CDR-L1 of E2M | artificial | RSSTGAVTSGYYPN |
| 185 | CDR-L2 of E2M | artificial | ATDMRPS |
| 186 | CDR-L3 of E2M | artificial | ALWYSNRWV |
| 187 | CDR-H1 of E2M | artificial | GYAMN |
| 188 | CDR-H2 of E2M | artificial | RIRSKYNNYATYYADSVKE |
| 189 | CDR-H3 of E2M | artificial | HRNFGNSYLSWFAY |
| 190 | CDR-L1 of F70 | artificial | GSSTGAVTSGYYPN |
| 191 | CDR-L2 of F70 | artificial | GTKFLAP |
| 192 | CDR-L3 of F70 | artificial | ALWYSNRWV |
| 193 | CDR-H1 of F70 | artificial | VYAMN |
| 194 | CDR-H2 of F70 | artificial | RIRSKYNNYATYYADSVKK |
| 195 | CDR-H3 of F70 | artificial | HGNFGNSYISWWAY |
| 196 | CDR-L1 of F12Q | artificial | GSSTGAVTSGNYPN |
| 197 | CDR-L2 of F12Q | artificial | GTKFLAP |
| 198 | CDR-L3 of F12Q | artificial | VLWYSNRWV |
| 199 | CDR-H1 of F12Q | artificial | SYAMN |
| 200 | CDR-H2 of F12Q | artificial | RIRSKYNNYATYYADSVKG |
| 201 | CDR-H3 of F12Q | artificial | HGNFGNSYVSWWAY |
| 202 | CDR-L1 of I2C | artificial | GSSTGAVTSGNYPN |
| 203 | CDR-L2 of I2C | artificial | GTKFLAP |
| 204 | CDR-L3 of I2C | artificial | VLWYSNRWV |
| 205 | CDR-H1 of I2C | artificial | KYAMN |
| 206 | CDR-H2 of I2C | artificial | RIRSKYNNYATYYADSVKD |
| 207 | CDR-H3 of I2C | artificial | HGNFGNSYISYWAY |
| 208 | I2C - VH | | EVQLVESGGGLVQPGGSLKLSCAASGFTFNKYAMNWVRQAPGKGLEWVARIRSKYNNYATY |
| | | | YADSVKDRFTISRDDSKNTAYLQMNNLKTEDTAVYYCVRHGNFGNSYISYWAYWGQGTLVTV |
| | | | SS |
| 209 | I2C - VL | | QTVVTQEPSLTVSPGGTVTLTCGSSTGAVTSGNYPNWVQQKPGQAPRGLIGGTKFLAPGTPARF |
| | | | SGSLLGGKAALTLSGVQPEDEAEYYCVLWYSNRWVFGGGTKLTVL |

1-14. (canceled)

15. A method for the treating (i) myeloid leukemia selected from relapsed/refractory AML (R/R AML) and AML with minimal residual disease (MRD) or (ii) myelodysplastic syndrome (MDS) in a patient in need thereof comprising administering a bispecific construct comprising a first binding domain specifically binding to CD33 and a second binding domain specifically binding to CD3 in one or more treatment cycles, wherein the at least one treatment cycle comprises more than 14 days of administration of the bispecific construct in at least three different dosages applying at least two dosage steps,
wherein the bispecific construct is administered in one treatment cycle according to a schedule comprising the following steps:
(a) administering a first dosage of the bispecific construct of at least 10 μg per day in the treatment of R/R AML or at least 30 μg per day in the treatment of MRD or MDS, followed by
(b) administering a second dosage of the bispecific construct, wherein said second dosage is at least 240 μg per day and/or preferably exceeds said first dose at least 10-fold in the treatment of R/R AML or at least 8-fold in the treatment of MRD or MDS and/or wherein the delta between the first and the second dosage is preferably at least 50 μg per day, preferably at least 100, 150, 200, 250, 300, 350, 360 or at most 400 μg per day, followed by
(c) administering a third dosage of the bispecific construct, wherein said third dosage is at least 600 per day and/or preferably exceeds said second dosage at most three-fold and/or wherein the delta between the second and the third dosage is preferably at least 50 μg per day, preferably at least 100, 150, 200, 250, 300, 350, 360, or at most 400 μg per day in the treatment of R/R AML, and wherein said third dosage is in the range of 600 to 1600 μg/d used in the treatment of MRD or MDS, preferably followed by
(d) administering a forth dosage of the bispecific construct preferably wherein the bispecific construct used in the treatment of R/R AML, wherein said optional forth dose is at least 720 μg per day and/or exceeds said third dosage and/or wherein the delta between the fourth and the fifth dosage is at least 50 μg per day, preferably at least 100, 150, 200, 250, 300, 350, 360 or at most 400 μg per day, optionally followed by
(e) administering a fifth dosage of the bispecific construct preferably wherein the bispecific construct used in the treatment of R/R AML, wherein said optional fifth dose is at least 960 μg per day and/or exceeds said fourth dosage and/or wherein the delta between the fourth and the fifth dosage is at least 50 μg per day, preferably at least 100, 150, 200, 250, 300, 350, 360, or at most 400 μg per day, optionally followed by
(f) administering a sixth dosage of the bispecific construct preferably wherein the bispecific construct is used in the treatment of R/R AML, wherein said optional sixth dose is at least 1200 μg per day and/or exceeds said fifth dosage and/or wherein the delta between the firth and the sixth dosage is at least 50 μg per day, preferably at least 100, 150, 200, 250, 300, 350, 360, or at most 400 μg per day.

16. The method according to claim 15, wherein the time of administering the bispecific construct in one treatment cycle is at least 15 days, preferably 15 to 60 days, more preferably 28 to 56 days, most preferably 28 days wherein the bispecific construct is used in the treatment of R/R AML or MRD AML or 56 days wherein the bispecific construct is used in the treatment MDS.

17. The method according to claim 15, preferably wherein the bispecific construct is for use in the treatment of R/R AML wherein the first dosage in step (a) is at least 10 μg per day, preferably in the range of 10 to 20 μg per day, preferably 10 μg per day, the second dosage in step (b) is at least 240 μg per day, preferably in the range of 240 to 600 μg per day, the third dosage in step (c) of at least 600 μg per day, preferably in the range of 600 to 1000 μg per day, and preferably the forth dosage in step (d) of at least 720 μg per day, preferably 720 to 1600 μg per day, more preferably in the range of 960 to 1080 μg per day, more preferably 960 μg per day, optionally the fifth dosage in step (e), of at least 960 μg per day, preferably at least 1200 or 1300 μg per day, and optionally the sixth dosage in step (f), of at least 1200 μg per day, preferably at least 1300 or 1600 μg per day.

18. The method according to claim 15, wherein the period of administration of the first dosage in step (a) is 1 to 5 days, preferably 1, 2 or 3 days (2 days in particular where used in the treatment of MRD), the period of administration of the second dosage in step (b) is 2 to 5 days, preferably 2 or 3 or 5 days (5 days in particular where used in the treatment of MRD), and the period of administration of the third and the optional forth dose in step (c) and optional step (d) is 7 to 52 days, preferably 14 to 23 days, more preferably 21, 22 or 23, where used in the treatment of R/R AML or MRD or 52 days where used in the treatment of MDS.

19. The method according to claim 15, wherein the treatment of the myeloid leukemia or MDS comprises two or more treatment cycles, preferably 2, 3, 4, 5, 6 or 7 treatment cycles, whereof at least 1, 2, 3, 4, 5, 6 or 7 treatment cycles comprise more than 14 days of bispecific construct administration.

20. The method according to claim 15,
wherein the treatment is followed by the period without administration of the bispecific construct, preferably at least 1, 2, 3, 4, 5, 6, 7, 8, 9, 10, 11, 12, 13, or 14 days without treatment.

21. The method according to claim 15,
wherein the treatment is followed by the period of at least 14 days without administration of the bispecific construct, preferably where the bispecific construct is for use in the treatment of MDS.

22. The method according to claim 15,
wherein only the first cycle of the treatment comprises the administration according to step (a), whereas the following cycles start with the dose according to step (b).

23. The method according to claim 15,
wherein the construct is a single chain bispecific construct.

24. The method according to claim 15, wherein the first binding domain of the bispecific construct comprises groups of six CDRs selected from the group consisting of SEQ ID NOs: 10 to 12 and 14 to 16, 22 to 24 and 26 to 28, 34 to 36 and 38 to 40, 46 to 48 and 50 to 52, 58 to 60 and 62 to 64, 70 to 72 and 74 to 76, 82 to 84 and 86 to 88, 94 to 96 an 98 to 100, preferably 94 to 96 an 98 to 100.

25. The method according to 15, wherein
the second binding domain of the bispecific construct comprises groups of six CDRs selected from the group consisting of SEQ ID NOs: 148-153, 154-159, 160-165, 166-171, 172-177, 178-183, 184-189, 190-195, 196-201 and 202-207, preferably 202-207.

26. The method according to 15, wherein
the first binding domain of the bispecific construct comprises groups of six CDRs selected from the group consisting of SEQ ID NOs: 94 to 96 or 98 to 100 and the second binding domain of the bispecific construct comprises groups of six CDRs selected from the group consisting of SEQ ID NOs: 202-207.

27. The method according to 15, wherein
the first binding domain of the bispecific construct comprises a VH of SEQ ID NO 93 and a VL of SEQ ID NO 97, and wherein the second binding domain of the bispecific construct comprises a VH of SEQ ID NO 208 and a VL of SEQ ID NO 209.

28. The method according to 15, wherein
the bispecific construct is a single chain construct comprising an amino acid sequence selected from the group consisting of SEQ ID NOs: 18, 19, 20, 30, 31, 32, 42, 43, 44, 54, 55, 56, 66, 67, 68, 78, 79, 80, 90, 91, 92, 102, 103, 104, 105, 106, 107 and 108, preferably selected from the group consisting of SEQ ID NOs: 104, 105, 106, 107 and 108, more preferably SEQ ID NO: 104.

29. (canceled)


SEQUENCE LISTING (209 sequences)

SEQ ID NO: 1 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 2 - protein, 27 aa, Homo sapiens
QDGNEEMGGITQTPYKVSISGTTVILT

SEQ ID NO: 3 - protein, 96 aa, Callithrix jacchus
QDGNEEMGDTTQNPYKVSISGTTVTLTCPRYDGHEIKWLVNSQNKEGHEDHLLLEDFSEMEQSGYYACLSKETPAEEASHYLYLKARVCENCVEVD

SEQ ID NO: 4 - protein, 27 aa, Callithrix jacchus
QDGNEEMGDTTQNPYKVSISGTTVTLT

SEQ ID NO: 5 - protein, 96 aa, Saguinus oedipus; identical to SEQ ID NO: 3

SEQ ID NO: 6 - protein, 27 aa, Saguinus oedipus
QDGNEEMGDTTQNPYKVSISGTTVTLT

SEQ ID NO: 7 - protein, 96 aa, Saimiri sciureus
QDGNEEIGDTTQNPYKVSISGTTVTLTCPRYDGQEIKWLVNDQNKEGHEDHLLLEDFSEMEQSGYYACLSKETPTEEASHYLYLKARVCENCVEVD

SEQ ID NO: 8 - protein, 27 aa, Saimiri sciureus
QDGNEEIGDTTQNPYKVSISGTTVTLT

SEQ ID NO: 9 - protein, 122 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSS

SEQ ID NO: 10 - protein, 5 aa, Artificial
  Synthetic peptide
NYGMN

SEQ ID NO: 11 - protein, 17 aa, Artificial
  Synthetic peptide
WINTYTGEPTYADDFKG

SEQ ID NO: 12 - protein, 13 aa, Artificial
  Synthetic peptide
WSWSDGYYVYFDY

SEQ ID NO: 13 - protein, 113 aa, Artificial
  Synthetic peptide
DIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 14 - protein, 17 aa, Artificial
  Synthetic peptide
KSSQSVLDSSKNKNSLA

SEQ ID NO: 15 - protein, 7 aa, Artificial
  Synthetic peptide
WASTRES

SEQ ID NO: 16 - protein, 9 aa, Artificial
  Synthetic peptide
QQSAHFPIT

SEQ ID NO: 17 - protein, 250 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 18 - protein, 505 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 19 - protein, 505 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 20 - protein, 505 aa, Artificial
  Synthetic peptide
QVQLVQSGAEVKKPGESVKVSCKASGYTFTNYGMNWVRQAPGQGLEWMGWINTYTGEPTYADDFKGRVTMSSDTSTSTAYLEINSLRSDDTAIYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 122 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSS

SEQ ID NO: 22 - protein, 5 aa, Artificial
  Synthetic peptide
NYGMN

SEQ ID NO: 23 - protein, 17 aa, Artificial
  Synthetic peptide
WINTYTGEPTYADDFKG

SEQ ID NO: 24 - protein, 13 aa, Artificial
  Synthetic peptide
WSWSDGYYVYFDY

SEQ ID NO: 25 - protein, 113 aa, Artificial; identical to SEQ ID NO: 13
  Synthetic peptide

SEQ ID NO: 26 - protein, 17 aa, Artificial
  Synthetic peptide
KSSQSVLDSSKNKNSLA

SEQ ID NO: 27 - protein, 7 aa, Artificial
  Synthetic peptide
WASTRES

SEQ ID NO: 28 - protein, 9 aa, Artificial
  Synthetic peptide
QQSAHFPIT

SEQ ID NO: 29 - protein, 250 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 30 - protein, 505 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 31 - protein, 505 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 32 - protein, 505 aa, Artificial
  Synthetic peptide
QVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 33 - protein, 122 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSS

SEQ ID NO: 34 - protein, 5 aa, Artificial
  Synthetic peptide
NYGMN

SEQ ID NO: 35 - protein, 17 aa, Artificial
  Synthetic peptide
WINTYTGEPTYADDFKG

SEQ ID NO: 36 - protein, 13 aa, Artificial
  Synthetic peptide
WSWSDGYYVYFDY

SEQ ID NO: 37 - protein, 113 aa, Artificial; identical to SEQ ID NO: 13
  Synthetic peptide

SEQ ID NO: 38 - protein, 17 aa, Artificial
  Synthetic peptide
KSSQSVLDSSKNKNSLA

SEQ ID NO: 39 - protein, 7 aa, Artificial
  Synthetic peptide
WASTRES

SEQ ID NO: 40 - protein, 9 aa, Artificial
  Synthetic peptide
QQSAHFPIT

SEQ ID NO: 41 - protein, 250 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 42 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 43 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 44 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTTDTSTSTAYMEIRNLRNDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 45 - protein, 122 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSS

SEQ ID NO: 46 - protein, 5 aa, Artificial
  Synthetic peptide
NYGMN

SEQ ID NO: 47 - protein, 17 aa, Artificial
  Synthetic peptide
WINTYTGEPTYADDFKG

SEQ ID NO: 48 - protein, 13 aa, Artificial
  Synthetic peptide
WSWSDGYYVYFDY

SEQ ID NO: 49 - protein, 113 aa, Artificial; identical to SEQ ID NO: 13
  Synthetic peptide

SEQ ID NO: 50 - protein, 17 aa, Artificial
  Synthetic peptide
KSSQSVLDSSKNKNSLA

SEQ ID NO: 51 - protein, 7 aa, Artificial
  Synthetic peptide
WASTRES

SEQ ID NO: 52 - protein, 9 aa, Artificial
  Synthetic peptide
QQSAHFPIT

SEQ ID NO: 53 - protein, 250 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 54 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 55 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 56 - protein, 505 aa, Artificial
  Synthetic peptide
QVQLVQSGAEVKKPGESVKVSCKASGYTFTNYGMNWVKQAPGQGLKWMGWINTYTGEPTYADDFKGRVTMTSDTSTSTAYMEISSLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 57 - protein, 122 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSS

SEQ ID NO: 58 - protein, 5 aa, Artificial
  Synthetic peptide
NYGMN

SEQ ID NO: 59 - protein, 17 aa, Artificial
  Synthetic peptide
WINTYTGETNYADKFQG

SEQ ID NO: 60 - protein, 13 aa, Artificial
  Synthetic peptide
WSWSDGYYVYFDY

SEQ ID NO: 61 - protein, 113 aa, Artificial
  Synthetic peptide
DIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIK

SEQ ID NO: 62 - protein, 17 aa, Artificial
  Synthetic peptide
KSSQSVLDSSTNKNSLA

SEQ ID NO: 63 - protein, 6 aa, Artificial
  Synthetic peptide
WASTRE

SEQ ID NO: 64 - protein, 9 aa, Artificial
  Synthetic peptide
QQSAHFPIT

SEQ ID NO: 65 - protein, 250 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIK

SEQ ID NO: 66 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 67 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 68 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSMTVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 69 - protein, 122 aa, Artificial; identical to SEQ ID NO: 57
  Synthetic peptide

SEQ ID NO: 70 - protein, 5 aa, Artificial
  Synthetic peptide
NYGMN

SEQ ID NO: 71 - protein, 17 aa, Artificial
  Synthetic peptide
WINTYTGETNYADKFQG

SEQ ID NO: 72 - protein, 13 aa, Artificial
  Synthetic peptide
WSWSDGYYVYFDY

SEQ ID NO: 73 - protein, 113 aa, Artificial
  Synthetic peptide
DIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIK

SEQ ID NO: 74 - protein, 17 aa, Artificial
  Synthetic peptide
KSSQSVLDSSTNKNSLA

SEQ ID NO: 75 - protein, 7 aa, Artificial
  Synthetic peptide
WASTRES

SEQ ID NO: 76 - protein, 9 aa, Artificial
  Synthetic peptide
QQSAHFPIT

SEQ ID NO: 77 - protein, 250 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIK

SEQ ID NO: 78 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 79 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 80 - protein, 505 aa, Artificial
  Synthetic peptide
QVQLVQSGAEVKKPGESVKVSCKASGYTFTNYGMNWVKQAPGQGLEWMGWINTYTGETNYADKFQGRVTFTSDTSTSTAYMELRNLKSDDTAVYYCARWSWSDGYYVYFDYWGQGTTVTVSSGGGGSGGGGSGGGGSDIVMTQSPDSLSVSLGERTTINCKSSQSVLDSSTNKNSLAWYQQKPGQPPKLLLSWASTRESGIPDRFSGSGSGTDFTLTIDSLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 122 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSS

SEQ ID NO: 82 - protein, 5 aa, Artificial
  Synthetic peptide
NYGMN

SEQ ID NO: 83 - protein, 17 aa, Artificial
  Synthetic peptide
WINTYTGEPTYADKFQG

SEQ ID NO: 84 - protein, 13 aa, Artificial
  Synthetic peptide
WSWSDGYYVYFDY

SEQ ID NO: 85 - protein, 113 aa, Artificial
  Synthetic peptide
DIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIK

SEQ ID NO: 86 - protein, 17 aa, Artificial
  Synthetic peptide
KSSQSVLDSSNNKNSLA

SEQ ID NO: 87 - protein, 7 aa, Artificial
  Synthetic peptide
WASTRES

SEQ ID NO: 88 - protein, 9 aa, Artificial
  Synthetic peptide
QQSAHFPIT

SEQ ID NO: 89 - protein, 250 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIK

SEQ ID NO: 90 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 91 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 92 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLRSDDTAVYYCARWSWSDGYYVYFDYWGQGTTVTVSSGGGGSGGGGSGGGGSDIVMTQSPDSLTVSLGERTTINCKSSQSVLDSSNNKNSLAWYQQKPGQPPKLLLSWASTRESGIPDRFSGSGSGTDFTLTIDGLQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 93 - protein, 122 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 94 - protein, 5 aa, Artificial
  Synthetic peptide
NYGMN

SEQ ID NO: 95 - protein, 17 aa, Artificial
  Synthetic peptide
WINTYTGEPTYADKFQG

SEQ ID NO: 96 - protein, 13 aa, Artificial
  Synthetic peptide
WSWSDGYYVYFDY

SEQ ID NO: 97 - protein, 113 aa, Artificial
  Synthetic peptide
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 98 - protein, 17 aa, Artificial
  Synthetic peptide
KSSQSVLDSSTNKNSLA

SEQ ID NO: 99 - protein, 7 aa, Artificial
  Synthetic peptide
WASTRES

SEQ ID NO: 100 - protein, 9 aa, Artificial
  Synthetic peptide
QQSAHFPIT

SEQ ID NO: 101 - protein, 250 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 102 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 103 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 505 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 105 - protein, 505 aa, Artificial
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLSGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 106 - protein, 505 aa, Artificial
  Synthetic peptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 107 - protein, 505 aa, Artificial
  Synthetic peptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSGGGGSQVQLVQSGAEVKKPGASVKVSCKASGYTFTNYGMNWVKQAPGQGLKWMGWINTYTGEPTYADDFKGRVTMTSDTSTSTAYLELHNLRSDDTAVYYCARWSWSDGYYVYFDYWGQGTTVTVSSGGGGSGGGGSGGGGSDIVMTQSPDSLTVSLGERTTINCKSSQSVLDSSKNKNSLAWYQQKPGQPPKLLLSWASTRESGIPDRFSGSGSGTDFTLTIDSLQPEDSATYYCQQSAHFPITFGQGTRLEIK

SEQ ID NO: 108 - protein, 511 aa, Artificial
  Synthetic peptide
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 109 - protein, 585 aa, Homo sapiens [sequence omitted]

SEQ ID NO: 110 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 111 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 112 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 113 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 114 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 115 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 116 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 117 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 118 - protein, 585 aa, Artificial
  Synthetic peptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 119 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 120 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 121 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 122 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 123 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 124 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 125 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 126 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 127 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 128 - protein, 585 aa, Artificial
  Synthetic peptide
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 129 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 130 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAGTFTFHADICTLSEKERQIKKQTALVELVKHKPKATKEQLKAAMDDFAAFVEKCCKADDKETCFAEEGKKLVAASQAALGL

SEQ ID NO: 131 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 132 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 133 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALGVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 134 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDKFAAFVEKCCKADDKETCFAEEGPKLVAASQAALGL

SEQ ID NO: 135 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPHLVAASQAALGL

SEQ ID NO: 136 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPHLVAASKAALGL

SEQ ID NO: 137 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDKFAAFVEKCCKADDKETCFAEEGPKLVAASKAALGL

SEQ ID NO: 138 - protein, 585 aa, Artificial
  Synthetic peptide
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALDVDETYVPKEFNAETFTFHADICTLSEKERQIKKQTALVELVKHKPKATKEQLKAVMDDFAAFVEKCCKADDKETCFAEEGPKLVAASKAALGL

SEQ ID NO: 139 - protein, 4 aa, Artificial
  Peptide linker
GGGG

SEQ ID NO: 140 - protein, 5 aa, Artificial
  Peptide linker
GGGGS

SEQ ID NO: 141 - protein, 5 aa, Artificial
  Peptide linker
GGGGQ

SEQ ID NO: 142 - protein, 6 aa, Artificial
  Peptide linker
PGGGGS

SEQ ID NO: 143 - protein, 6 aa, Artificial
  Peptide linker
PGGDGS

SEQ ID NO: 144 - protein, 6 aa, Artificial
  Peptide linker
SGGGGS

SEQ ID NO: 145 - protein, 9 aa, Artificial
  Peptide linker
GGGGSGGGS

SEQ ID NO: 146 - protein, 10 aa, Artificial
  Peptide linker
GGGGSGGGGS

SEQ ID NO: 147 - protein, 15 aa, Artificial
  Peptide linker
GGGGSGGGGSGGGGS

SEQ ID NO: 148 - protein, 14 aa, Artificial
  synthetic peptide
GSSTGAVTSGYYPN

SEQ ID NO: 149 - protein, 7 aa, Artificial
  synthetic peptide
GTKFLAP

SEQ ID NO: 150 - protein, 9 aa, Artificial
  synthetic peptide
ALWYSNRWV

SEQ ID NO: 151 - protein, 5 aa, Artificial
  synthetic peptide
IYAMN

SEQ ID NO: 152 - protein, 19 aa, Artificial
  synthetic peptide
RIRSKYNNYATYYADSVKS

SEQ ID NO: 153 - protein, 14 aa, Artificial
  synthetic peptide
HGNFGNSYVSFFAY

SEQ ID NO: 154 - protein, 14 aa, Artificial
  synthetic peptide
GSSTGAVTSGYYPN

SEQ ID NO: 155 - protein, 7 aa, Artificial
  synthetic peptide
GTKFLAP

SEQ ID NO: 156 - protein, 9 aa, Artificial
  synthetic peptide
ALWYSNRWV

SEQ ID NO: 157 - protein, 5 aa, Artificial
  synthetic peptide
KYAMN

SEQ ID NO: 158 - protein, 19 aa, Artificial
  synthetic peptide
RIRSKYNNYATYYADSVKD

SEQ ID NO: 159 - protein, 14 aa, Artificial
  synthetic peptide
HGNFGNSYISYWAY

SEQ ID NO: 160 - protein, 14 aa, Artificial
  synthetic peptide
GSSTGAVTSGYYPN

SEQ ID NO: 161 - protein, 7 aa, Artificial
  synthetic peptide
GTKFLAP

SEQ ID NO: 162 - protein, 9 aa, Artificial
  synthetic peptide
ALWYSNRWV

SEQ ID NO: 163 - protein, 5 aa, Artificial
  synthetic peptide
SYAMN

SEQ ID NO: 164 - protein, 19 aa, Artificial
  synthetic peptide
RIRSKYNNYATYYADSVKG

SEQ ID NO: 165 - protein, 14 aa, Artificial
  synthetic peptide
HGNFGNSYLSFWAY

SEQ ID NO: 166 - protein, 14 aa, Artificial
  synthetic peptide
GSSTGAVTSGYYPN

SEQ ID NO: 167 - protein, 7 aa, Artificial
  synthetic peptide
GTKFLAP

SEQ ID NO: 168 - protein, 9 aa, Artificial
  synthetic peptide
ALWYSNRWV

SEQ ID NO: 169 - protein, 5 aa, Artificial
  synthetic peptide
RYAMN

SEQ ID NO: 170 - protein, 19 aa, Artificial
  synthetic peptide
RIRSKYNNYATYYADSVKG

SEQ ID NO: 171 - protein, 14 aa, Artificial
  synthetic peptide
HGNFGNSYLSYFAY

SEQ ID NO: 172 - protein, 14 aa, Artificial
  synthetic peptide
RSSTGAVTSGYYPN

SEQ ID NO: 173 - protein, 7 aa, Artificial
  synthetic peptide
ATDMRPS

SEQ ID NO: 174 - protein, 9 aa, Artificial
  synthetic peptide
ALWYSNRWV

SEQ ID NO: 175 - protein, 5 aa, Artificial
  synthetic peptide
VYAMN

SEQ ID NO: 176 - protein, 19 aa, Artificial
  synthetic peptide
RIRSKYNNYATYYADSVKK

SEQ ID NO: 177 - protein, 14 aa, Artificial
  synthetic peptide
HGNFGNSYLSWWAY

SEQ ID NO: 178 - protein, 14 aa, Artificial
  synthetic peptide
GSSTGAVTSGYYPN

SEQ ID NO: 179 - protein, 7 aa, Artificial
  synthetic peptide
GTKFLAP

SEQ ID NO: 180 - protein, 9 aa, Artificial
  synthetic peptide
ALWYSNRWV

SEQ ID NO: 181 - protein, 5 aa, Artificial
  synthetic peptide
KYAMN

SEQ ID NO: 182 - protein, 19 aa, Artificial
  synthetic peptide
RIRSKYNNYATYYADSVKS

SEQ ID NO: 183 - protein, 14 aa, Artificial
  synthetic peptide
HGNFGNSYTSYYAY

SEQ ID NO: 184 - protein, 14 aa, Artificial
  synthetic peptide
RSSTGAVTSGYYPN

SEQ ID NO: 185 - protein, 7 aa, Artificial
  synthetic peptide
ATDMRPS

SEQ ID NO: 186 - protein, 9 aa, Artificial
  synthetic peptide
ALWYSNRWV

SEQ ID NO: 187 - protein, 5 aa, Artificial
  synthetic peptide
GYAMN

SEQ ID NO: 188 - protein, 19 aa, Artificial
  synthetic peptide
RIRSKYNNYATYYADSVKE

SEQ ID NO: 189 - protein, 14 aa, Artificial
  synthetic peptide
HRNFGNSYLSWFAY

SEQ ID NO: 190 - protein, 14 aa, Artificial
  synthetic peptide
GSSTGAVTSGYYPN

SEQ ID NO: 191 - protein, 7 aa, Artificial
  synthetic peptide
GTKFLAP

SEQ ID NO: 192 - protein, 9 aa, Artificial
  synthetic peptide
ALWYSNRWV

SEQ ID NO: 193 - protein, 5 aa, Artificial
  synthetic peptide
VYAMN

SEQ ID NO: 194 - protein, 19 aa, Artificial
  synthetic peptide
RIRSKYNNYATYYADSVKK

SEQ ID NO: 195 - protein, 14 aa, Artificial
  synthetic peptide
HGNFGNSYISWWAY

SEQ ID NO: 196 - protein, 14 aa, Artificial
  synthetic peptide
GSSTGAVTSGNYPN

SEQ ID NO: 197 - protein, 7 aa, Artificial
  synthetic peptide
GTKFLAP

SEQ ID NO: 198 - protein, 9 aa, Artificial
  synthetic peptide
VLWYSNRWV

SEQ ID NO: 199 - protein, 5 aa, Artificial
  synthetic peptide
SYAMN

SEQ ID NO: 200 - protein, 19 aa, Artificial
  synthetic peptide
RIRSKYNNYATYYADSVKG

SEQ ID NO: 201 - protein, 14 aa, Artificial
  synthetic peptide
HGNFGNSYVSWWAY

SEQ ID NO: 202 - protein, 14 aa, Artificial
  synthetic peptide
GSSTGAVTSGNYPN

SEQ ID NO: 203 - protein, 7 aa, Artificial
  synthetic peptide
GTKFLAP

SEQ ID NO: 204 - protein, 9 aa, Artificial
  synthetic peptide
VLWYSNRWV

SEQ ID NO: 205 - protein, 5 aa, Artificial
  synthetic peptide
KYAMN

SEQ ID NO: 206 - protein, 19 aa, Artificial
  synthetic peptide
RIRSKYNNYATYYADSVKD

SEQ ID NO: 207 - protein, 14 aa, Artificial
  synthetic peptide
HGNFGNSYISYWAY

SEQ ID NO: 208 - protein, 125 aa, Artificial Sequence
  Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 209 - protein, 109 aa, Artificial Sequence
  Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL